(19) United States
(12) Patent Application Publication
(10) Pub. No.: US 2024/0374763 A1
(43) Pub. Date: Nov. 14, 2024

(54) DIAGNOSIS AND MONITORING USING EXTRADOMAIN-B FIBRONECTIN TARGETED PROBES

(71) Applicant: Case Western Reserve University, Cleveland, OH (US)

(72) Inventors: Zheng-Rong LU, Cleveland, OH (US); Nadia AVAT, Cleveland, OH (US); Ryan HALL, Cleveland, OH (US); Amita M. VAIDYA, Cleveland, OH (US); Peter QIAO, Cleveland, OH (US)

(21) Appl. No.: 17/928,764

(22) PCT Filed: Jun. 4, 2021
(86) PCT No.: PCT/US2021/035852

§ 371 Date: Nov. 30, 2022

Related U.S. Application Data
(60) Provisional application No. 63/034,520, filed on Jun. 4, 2020.
(60) Provisional application No. 63/170,746, filed on Apr. 5, 2021.

(51) Int. Cl. A61K 49/14; A61K 49/12; A61K 49/18; A61K 51/08; A61P 35/00

(52) U.S. Cl. CPC A61K 49/14; A61K 49/126; A61K 49/1824; A61K 51/088; A61P 35/00

(57) ABSTRACT

A method of detecting drug-resistant cancer in a subject is described. The method includes contacting a tissue of the subject with an effective amount of a molecular probe, detecting the amount of the molecular probe present in the tissue, comparing the amount of molecular probe detected to a control value, and detecting drug-resistant cancer in the subject if the amount of the molecular probe present in the tissue is higher than the control value. The molecular probe includes the following formula: P-L-C wherein P is a EDB-FN targeting peptide, C is a contrast agent; and L is a non-peptide linker that covalently links the peptide to the contrast agent. Methods of monitoring the treatment of drug resistant cancer are also described.

### CROSS-REFERENCE TO RELATED APPLICATIONS

This application claims priority to U.S. Provisional Application No. 63/034,520, filed on Jun. 4, 2020, and U.S. Provisional Application No. 63,170,746, filed on Apr. 5, 2021, both of which are incorporated herein by reference.

### GOVERNMENT FUNDING

This invention was made with government support under CA211762 and CA194518 awarded by National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Mar. 31, 2021, is named CWR-029593 WO ORD_ST25.txt and is 7,841 bytes in size.

### BACKGROUND

Cancer is the second most common cause of death in men and women combined in the US. Accurate early diagnosis of cancer is critical to initiate precise, personalized therapeutic interventions and to improve the survival and quality of life of the patients diagnosed with the disease. Although treatable with chemotherapy and surgery in early stages, development of multidrug resistance can lead to relapse and distant metastases, which are incurable. Accurate non-invasive assessment of therapeutic efficacy early on in the treatment regimen can potentially increase cancer survivability and reduce cost of care. While blood markers and imaging modalities like PET-CT, MRI, and ultrasound are commonly used for diagnosis and assessment of the disease, they are unable to distinguish between drug-resistant tumors and sensitive ones.

Extradomain-B fibronectin (EDB-FN) is an oncofetal isoform of fibronectin. This extracellular matrix oncoprotein is significantly upregulated in a plethora of neoplasms including colorectal cancer, and is associated with epithelial-to-mesenchymal transition (EMT), cancer cell sternness, proliferation, angiogenesis, and metastasis, all of which reflect tumor aggressiveness. Clinical studies demonstrate the presence of EDB-FN in patients with lung, brain, colorectal, and ovarian cancers. The overexpression of EDB-FN is also correlated with histological grade in mammary tumors and with poor survival in oral carcinoma patients, suggesting its potential role as a marker for multiple neoplasms.

An added layer of complication is that even among the same cancer type, EDB-FN expression profiles are distinct and specific to the molecular and functional characteristics of the cells or tissues. For example, using an EDB-FN-specific peptide probe, ZD2-Cy5.5, the inventors previously showed that invasive cancer cell lines, e.g., PC3 (prostate) and MDA-MB-231 (hormone receptor-negative breast cancer), are EDB-FN-rich, while the less invasive cancer cell lines, e.g., LNCaP (prostate) and MCF7 (hormone receptor-positive breast) exhibit significantly lower EDB-FN levels. This differential expression of EDB-FN was exploited for differentially diagnosing invasive prostate and breast cancer tumors from the non-invasive xenografts using EDB-FN-targeted MRI contrast agents, specifically MT218 [ZD2-N3-Gd(HP-DO3A)]. However, there remains a need for non-invasive assessment of drug resistance, monitoring of treatment response, and active surveillance of drug-resistant cancer.

### SUMMARY

The inventors have determined that EDB-FN is overexpressed in highly invasive drug-resistant colorectal cancer (CRC) cells and tumors. Consequently, MRMI of EDB-FN using MT218 can facilitate effective non-invasive detection and differential diagnosis of drug-resistant CRC xenograft models. Moreover, MRMI by MT218 can also be used to monitor therapeutic efficacy of targeted drugs, including MK2206.HCl, Paclitaxel, etc. on drug-resistant CRC tumors.

In one aspect, the present invention provides a method of detecting drug-resistant cancer in a subject. The method includes the steps of contacting a tissue of the subject with an effective amount of a molecular probe comprising the formula P-L-C, wherein: P is a peptide that includes an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9; C is a contrast agent; and L is a non-peptide linker that covalently links the peptide to the contrast agent, the linker including a carboxylic acid that forms a carboxamide with an amine of the peptide or a maleimide that forms a thioester bond with a cysteine reside of the peptide or a maleimide that forms a thioester with a cysteine residue of the peptide, detecting the amount of the molecular probe present in the tissue, comparing the amount of molecular probe detected to a control value, and detecting drug-resistant cancer in the subject if the amount of the molecular probe present in the tissue is higher than the control value.

In another aspect, the present invention provides method of monitoring the treatment of drug resistant cancer. The method includes the steps of contacting a tissue of a subject undergoing treatment of drug resistant cancer with an effective amount of a molecular probe comprising the formula P-L-C for a first time, wherein: P is a peptide that includes an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9; C is a contrast agent; and L is a non-peptide linker that covalently links the peptide to the contrast agent, the linker including a carboxylic acid that forms a carboxamide with an amine of the peptide or a maleimide that forms a thioester bond with a cysteine reside of the peptide or a maleimide that forms a thioester with a cysteine residue of the peptide; detecting a first amount of the molecular probe present in the tissue; contacting the tissue of the subject for a second time with an effective amount of a molecular probe comprising the formula P-L-C; detecting a second amount of the molecular probe present in the tissue; and comparing the first amount and the second amount of the molecular probe to monitor the treatment of the drug resistant cancer in the subject.

In some embodiments, the cancer is breast cancer, oral cancer, pancreatic cancer, or prostate cancer. In further embodiments, the tissue is contacted in vivo. In additional embodiments, the drug resistant cancer is being treated with a chemotherapeutic agent.

In some embodiments, the molecular probe is a magnetic resonance imaging agent. In additional embodiments, the non-peptide linker of the molecular probe is a non-peptide aliphatic or heteroaliphatic linker. In further embodiments, the non-peptide linker of the molecular probe includes an alkylene dicarboxamide when covalently linking the peptide and contrast agent.

In some embodiments, the contrast agent of the molecular probe includes at least one of metal chelating agent or a metallofullerene. In further embodiments, the contrast agent of the molecular probe includes a metal chelating agent comprising at least one of diethylenetriaminepentaacetate (DTPA) or its derivatives, 1,4,7,10-tetraazadodecanetetraacetate (DOTA) and its derivatives, 1,4,7,10-tetraazadodecane-1,4,7-triacetate (DO3A) and its derivatives, ethylenediaminetetraacetate (EDTA) and its derivatives, 1,4,7,10-tetraazacyclotridecanetetraacetic acid (TRITA) and its derivatives, 1,4,8,11-tetraazacyclotetradecane-1,4,8,11-tetraacetic acid (TETA) and its derivatives, 1,4,7,10-tetraazadodecanetetramethylacetate (DOTMA) and its derivatives, 1,4,7,10-tetraazadodecane-1,4,7-trimethylacetate (DO3MA) and its derivatives, N,N′,N″,N′″-tetraphosphonatomethyl-1,4,7,10-tetraazacyclododecane (DOTP) and its derivatives, 1,4,7,10-tetraazacyclododecane-1,4,7,10-tetrakis(methylene methylphosphonic acid) (DOTMP) and its derivatives, 1,4,7,10-tetraazacyclododecane-1,4,7,10-tetrakis(methylene phenylphosphonic acid) (DOTPP) and its derivatives, or N,N′-ethylenedi-L-cysteine and its derivatives.

In some embodiments, the molecular probe has the formula:

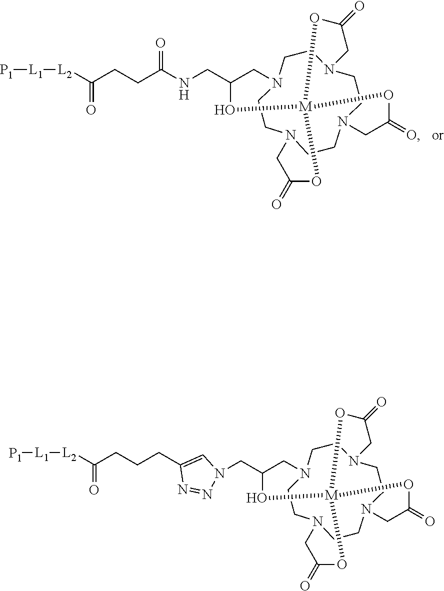

wherein: P₁ is a peptide having an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9; L₁ is an optional spacer; L₂ is an amino group of the peptide P₁ or the spacer; and M is a metal selected from the group consisting of Gd⁺³, Eu⁺³, Tm⁺³, Dy⁺³, Yb⁺³, Mn⁺², Fe⁺³, ⁵⁵Co, ⁶⁴Cu, ⁶⁷Cu, ⁴⁷Sc, ⁶⁶Ga ⁶⁸Ga, ⁹⁰Y, ⁹⁷Ru, ⁹⁹mTc, ¹¹¹h, ¹⁰⁹Pd ¹⁵³Sm, ¹⁷⁷Lu, ¹⁸⁶Re, and ¹⁸⁸Re; or salts thereof.

In further embodiments, L₁ comprises at least one of polyalkyleneoxide, polyvinyl alcohol, polyethylene glycol (PEG), polypropylene glycol (PPG), co-poly (ethylene/propylene) glycol, polyoxyethylene (POE), polyurethane, polyphosphazene, polysaccharides, dextran, polyvinylpyrrolidones, polyvinyl ethyl ether, polyacryl amide, polyacrylate, or polycyanoacrylates.

### BRIEF DESCRIPTION OF THE FIGURES

The accompanying drawings, which are incorporated in and constitute a part of this specification, illustrate some embodiments of the inventions, and together with the description, serve to explain principles of the inventions.

FIG. 1A-1H provides images showing that acquired drug resistance enhances migration and invasion of CRC cells. A. Morphological changes in 2D monolayer and 3D tumor spheroid cultures in DLD1-DR cells with acquired resistance to 5′-FU by parent DLD1 cells. B. Altered morphology in 2D monolayer and 3D tumor spheroid cultures in RKO-DR cells with acquired resistance to 5′-FU and CB-839 by parent RKO cells. Increased expression of drug resistance marker protein MDR1 and altered levels of EMT markers N-cad and E-cad in C, D. DLD1-DR and E, F. RKO-DR cells at protein and mRNA levels using western blotting and qRT-PCR, respectively. The western blot band intensities were normalized to the actin control, and the protein level changes were expressed as ratio of sensitive to resistant, next to the respective lanes. Increased invasion through Matrigel and migration measured using transwell assay in drug-resistant G. DLD1-DR and H. RKO-DR cells, compared to their parent counterparts. Cells were stained using 0.1% crystal violet. Bars indicate mean±sem. *p<0.05 using unpaired t-test. Scale bar=100 μm.

FIGS. 2A-2F provide graphs and images showing acquired drug resistance is associated with EDB-FN expression in CRC cells. CRC cells cultured in 3D Matrigel were stained with EDB-FN-specific peptide probe ZD2-Cy5.5 and nuclear stain Hoechst. CRC cells grown in 2D monolayer culture were harvested for RNA extraction and qRT-PCR analysis of mRNA expression. Drug-resistant DLD1-DR cells show A. increased ZD2-Cy5.5 binding, and 3-fold higher mRNA levels of B. EDB-FN and C. FN1, compared to DLD-1 cells. Drug-resistant RKO-DR cells show D. increased ZD2-Cy5.5 binding, and 4- and 1.5-fold higher mRNA levels of E. EDB-FN and F. FN1, compared to RKO cells. Bars indicate mean±sem. *p<0.05 using unpaired t-test. Scale bar=100 μm (100×) and 50 μm (200×).

FIGS. 3A-3J provide graphs and images showing Contrast-enhanced MRMI of EDB-FN using MT218 facilitates non-invasive assessment of drug resistance in CRC tumors. T1-weighted FSE coronal and axial images were obtained pre- and post-injection (25 min) of 40 μmol/kg dose of MT218 in CRC xenograft-bearing mice. Representative coronal and axial images of MRI show robust signal enhancement in A-B. DLD1-DR and C-D. RKO-DR tumors, compared to the non-resistant DLD-1 and RKO tumors (white arrows: tumors). Drug-resistant E-F. DLD1-DR and G-H. RKO-DR tumors exhibit significantly higher CNRs over DLD-1 and RKO tumors, respectively. (n=5 mice per group). Bars indicate mean±sem and dots indicate individual mice CNR. *p<0.05 using unpaired t-test. Post-mortem histology shows poorly differentiated mitotically active cells by H&E, and stronger staining with anti-EDB-FN antibody G4 in drug-resistant I. DLD1-DR and J. RKO-DR tumor sections over their respective non-resistant counterparts (red and black arrows denote EDB-FN in tumor cells and fibroblasts, respectively).

FIGS. 4A-4C provide graphs showing the timeline of therapeutic regimen and non-invasive monitoring of therapeutic efficacy in drug-resistant DLD1-DR tumor-bearing mice. A. Drug-resistant DLD1-DR tumors were subcutaneously implanted in the flanks of athymic nu/nu female mice. Tumor volumes were monitored weekly with Vernier caliper. Baseline MRMI for EDB-FN using MT218 was performed at week 1, when the tumors reached 100 mm3 volumes. Mice were randomized into DMSO-treated ‘Vehicle’ group and MK2206-treated ‘Treated’ group. At week 4, endpoint MRMI for EDB-FN using MT218 was performed and the experiment was terminated. B. Average tumor volumes increased from week 1 to 4 for each group. C. Tumor volumes for individual mice (numbered TM1-TM10) from both the Vehicle and Treated groups increased progressively from week 1 to 4. (n=5 mice per group). Bars indicate mean±sem and dots indicate individual mice tumor volumes. *p<0.05 using unpaired t-test.

FIGS. 5A-5E provide graphs and images showing MRMI of EDB-FN with MT218 facilitates non-invasive therapeutic monitoring of vehicle-treated mice bearing drug-resistant CRC tumors. T1-weighted 2D spin echo axial images from MRMI performed at A. Baseline (week 1) and B. Endpoint (week 4) with 40 μmol/kg dose MT218 in DLD1-DR-bearing mice treated with DMSO (TM: designation for individual mice in Vehicle group). Images were taken before and 25 min post-injection of MT218. C. CNRs for individual mice plotted for week 1 and week 4. D. Average CNRs for the Vehicle group did not change significantly from week 1 to week 4. E. Postmortem histology performed after week 4 shows poorly differentiated DLD1-DR tumor sections. Immunohistochemistry with anti-EDB-FN antibody G4 shows stronger staining in TM3 than TM10. (n=5 mice per group). Bars indicate mean±sem and dots indicate individual mice tumor volumes. *p<0.05 using unpaired t-test.

FIGS. 6A-6E provide graphs and images showing MRMI of EDB-FN with MT218 facilitates non-invasive therapeutic monitoring of MK2206-HCl-treated mice bearing drug-resistant CRC tumors. T1-weighted 2D spin echo axial images from MRMI performed at A. Baseline (week 1) and B. Endpoint (week 4) with 40 μmol/kg dose MT218 in DLD1-DR-bearing mice treated with 100 mg/kg MK2206-HCl (TM: designation for individual mice in Treated group). Images were taken before and 25 min post-injection of MT218. C. CNRs for individual mice plotted for week 1 and week 4. D. Average CNRs for the Treated group increased significantly from week 1 to week 4. E. Postmortem histology performed after week 4 shows poorly differentiated DLD1-DR tumor sections. Immunohistochemistry with anti-EDB-FN antibody G4 shows stronger staining in TM7 than TM9. (n=5 mice per group). Bars indicate mean±sem and dots indicate individual mice tumor volumes. *p<0.05 using unpaired t-test.

FIGS. 7A-7F provide graphs and images showing EDB-FN is overexpressed inhuman colon adenocarcinoma and is correlated with poor survival. Immunohistochemical staining using EDB-FN-specific antibody G4 shows strong EDB-FN expression in representative primary colon adenocarcinoma tissues from A. non-treated patients and B. treated patients, but negligible EDB-FN expression in the corresponding normal adjacent tissues (NAT). Immunohistochemical staining of representative metastatic liver specimens shows differential EDB-FN expression in C. non-treated and D. treated patients, with no expression in the corresponding normal adjacent liver tissue (NAT). Black and red arrows denote fibroblasts and tumor cells, respectively. Kaplan-Meier curves demonstrate that high EDB-FN levels correlate with E. poor overall survival (OS) and F. poor progression-free survival (PFS) of colon adenocarcinoma patients (*p=0.018 and *p=0.02 for Hazard ratio for OS and *p=0.025 and *p=0.028 for PFS using Log-rank test, i.e., Mantel-Cox test).

FIGS. 8A-8H provide graphs and images showing EDB-FN overexpression in invasive breast cancer. A. Differential gene expression analysis performed on patient data from the TCGA and GTEx databases shows significant overexpression of EDB-FN isoform (ENST00000432072.6) in breast tumor samples (BRCA, n=1084) compared to normal breast tissue samples (Nor, n=291). Box plot denotes log 2 (TPM+1) transcripts per million values and median with interquartile range, *p=6.57e-69 using ANOVA. B. Kaplan-Meier curves show overall survival analyses of breast cancer patients with EDB-FN expression (*p<0.05; *p for Hazard ratio=0.022 using Log-rank test, i.e., Mantel-Cox test). C. Immunohistochemical staining pattern of EDB-FN in primary breast tumors showing expression in fibroblasts (green), cancer cells (purple), and tumor stroma (red). Strong EDB-FN staining observed in D. breast tumors of patients 1 and 2, compared to normal adjacent tissue (NAT), E. lymph node metastases, and F. lung and brain metastases. G. qRT-PCR analysis exhibits significantly elevated levels of EDB-FN mRNA in triple-negative MDA-MB-468, BT549, MDA-MB-231, and Hs578T cells, compared to hormone receptor-positive MCF7 cells. 18S expression was used as standard. Bars denote mean±sem (n=3). **p=0.0013 using Kruskal-Wallis test. H. Comparison of the invasive potential of different subtypes of breast cancer cell lines by transwell assay. EDB-FN-overexpressing triple-negative cancer cells are more invasive than the EDB-FN-low MCF7 cells, evidenced by the increased number of crystal violet-stained cells invading through Matrigel-coated transwell inserts.

FIGS. 9A-9E provide images showing the altered features and enhanced migration in breast cancer cells with TGF-β treatment and drug resistance. MCF7 and MDA-MB-468 cells were cultured in 5 ng/mL TGF-β for 7-15 days to obtain MCF7-TGF-β and MDA-MB-468-TGF-β, cells respectively. MCF7-DR and MDA-MB-468-DR cells were obtained by inducing resistance to 500 nM Palbociclib and 100 nM Paclitaxel respectively. A. In 2D culture, MCF7-TGFβ and MCF7-DR cells show distinct morphological changes, with a more mesenchymal phenotype, while MDA-MB-468-TGFβ and MDA-MB-468-DR cells do not show visible morphological changes, compared to their parent lines. B. In 3D Matrigel culture, MCF7-TGFβ and MCF7-DR cells show increased ability to form spheroids, while MDA-MB-468-TGFβ and MDA-MB-468-DR cells form similar proliferative networks, compared to their respective parent counterparts. Western blot analysis for EMT markers shows protein expression of E-cadherin, N-cadherin, and Slug in C. MCF7-TGFβ and MCF7-DR and D. MDA-MB-468-TGFβ and MDA-MB-468-DR cells, compared to the parent MCF7 and MDA-MB-468 cells, respectively. E. Transwell assay shows higher invasive potential of TGF-β-treated and drug-resistant MCF7 and MDA-MB-468 cells, evidenced by the increase in number of crystal violet-stained migrated cells. Scale bar=100 μm.

FIGS. 10A-10F provide graphs and images showing increased EDB-FN expression in breast cancer cells with TGF-β treatment and drug resistance. A. ZD2-Cy5.5 staining of 3D cultures of breast cancer cells shows significantly increased EDB-FN expression in A. MCF7-TGF-β and MCF7-DR and B. MDA-MB-468-TGF-β and MDA-MB-468-DR cells, compared to the parent MCF7 and MDA-MB-468 cells, respectively. ZD2 peptide binding to EDB-FN was quantified in FIJI as the ratio of the pixel intensities of ZD2-Cy5.5 to that of Hoechst to show increased EDB-FN level in C. MCF7-TGF-β and MCF7-DR and D. MDA-MB-468-TGF-β and MDA-MB-468-DR cells, compared to the parent MCF7 and MDA-MB-468 cells. Bars denote mean±sem and dots denote independent replicates. *p<0.05 using Mann-Whitney U test. qRT-PCR analysis shows significantly upregulated mRNA expression of EDB-FN in E. MCF7-TGFβ and MCF7-DR and F. MDA-MB-468-TGFβ and MDA-MB-468-DR cells, compared to the parent MCF7 and MDA-MB-468 cells, respectively. Dots denote 3 technical replicates from 3 independent experiments, with lines at mean±sem. *p<0.05 using Mann-Whitney U test. Scale bars=100 μm.

FIGS. 11A-I1H provide graphs and images showing therapeutic ablation of AKT in invasive TGF-β-treated and drug-resistant breast cancer cells reduces their invasion and EDB-FN overexpression. A. TGF-β treatment and drug resistance upregulate phospho-AKT signaling and phosphorylation of SRp55 in MCF7 and MDA-MB-468 cells. B. Treatment of cells with AKT inhibitor MK2206-HCl (2 μM for MCF7 and 4 μM for MDA-MB-468 cells) for 2 days results in inhibition of phospho-AKT signaling in the invasive breast cancer cell lines. The SRp55 upregulation is diminished with MK2206-HCl-mediated depletion of phospho-AKT signaling, suggesting a potential role of SRp55 in the inclusion of EDB-FN exon. Inhibition of phospho-AKT signaling by MK2206-HCl demonstrates reduced invasive potential of the invasive C. MCF7 and D. MDA-MB-468 cell derivatives along with decreased expression of EDB-FN at E. and F. mRNA levels and G. and H. in 3D culture, respectively. Dots denote 2 technical replicates from 3 independent experiments, with lines at mean±sem. G-H. ZD2 peptide binding to EDB-FN was quantified in FIJI as the ratio of the pixel intensities of ZD2-Cy5.5 to that of Hoechst. Bars denote mean±sem and dots denote independent replicates. *p<0.05 using Mann-Whitney U test. Scale bars=100 μm.

FIGS. 12A-12G provide images showing the depletion of EDB-FN and SRp55 in invasive TGF-β-treated and drug-resistant breast cancer cells reduces their invasion. TGF-β-treated and drug-resistant MCF7 and MDA-MB-468 cells were treated with ECO/siNC (siN), ECO/siEDB (siE), and ECO/siSRSF6 (siS) nanoparticles at 100 nM [siRNA] and N/P=10. A. Knockdown of EDB-FN and SRSF6 results in downregulation of EDB-FN expression and SRp55 levels with a concomitant reduction in the invasive potential of the invasive TGF-β-treated and drug-resistant B. MCF7 and C. MDA-MB-468 cells. Treatment of invasive MDA-MB-468-DR cells with MK2206-HCl D. reduces EDB-FN expression and E. invasion. Overexpression of TGF-β in these MK2206-treated cells rescues EDB-FN expression as well as invasive potential of these cells. Treatment of invasive MCF7-DR cells with MK2206-HCl F. reduces EDB-FN expression and G. invasion. Overexpression of TGF-β in these MK2206-treated cells does not rescue EDB-FN expression or their invasive potential. Scale bar=100 μm.

FIGS. 13A-13D provide images showing the immunohistochemical staining of EDB-FN in human tissues with the EDB-FN-specific G4 antibody was (a) weak in normal tongue specimens (n=3), (b) strong in untreated primary OSCC specimens (n=7), (c) strong in OSCC metastatic specimens (n=2), and (d) moderate in neoadjuvant primary OSCC specimens (n=3). Scale bars in all panels: 200 μm.

FIGS. 14A and 14B provide images showing (a) Cells grown in 2D and 3D culture (n=3) showed high-risk features in HSC3 and SCC4 cells, but not CAL27 cells. (b) Transwell migration and invasion assays (n=3) demonstrated high invasive potential of HSC3 and SCC4 cells, but not CAL27 cells.

FIGS. 15A-15E provide graphs and images showing (a) qRT-PCR mRNA analysis (n=3) and (b) western blot protein analysis (n=3) revealed differential and upregulated expression of EDB-FN in aggressive HSC3 and SCC4 cells. (c) Peptide binding study of spheroids with EDB-FN-specific ZD2-Cy5.5 (n=3) showed strong staining in HSC3 and SCC4 spheroids, but not CAL27 spheroids. Quantification of confocal images revealed (d) larger spheroids and (e) stronger ZD2-Cy5.5 staining in HSC3 and SCC4 spheroids in accordance with their EDB-FN expression levels. Data was analyzed with One-Way ANOVA and Fisher's least significant difference post-hoc tests (*, p<0.05; **, p<0.01; ***, p<0.001; n.s., p>0.05).

FIGS. 16A-16C provide images showing (a) CAL27 tumors (n=5) showed slightly less enhancement with MT218 than gadoteridol. (b) HSC3 tumors (n=4) showed slightly more enhancement with MT218 than gadoteridol. (c) SCC4 tumors (n=4) showed substantially more enhancement with MT218 than gadoteridol. MT218 and gadoteridol were used at doses of 0.04 mmol/kg and 0.10 mmol/kg, respectively.

FIGS. 17A-17I provide graphs showing that CNR analysis of contrast-enhanced tumors revealed (a) significant differential enhancement between CAL27, HSC3, and SCC4 tumors with MT218 in accordance with their EDB-FN expression, while (b) gadoteridol showed no difference in enhancement between the three models. Enhancement with MT218 was (c) generally lower than gadoteridol in CAL27 tumors, (d) generally higher than gadoteridol in HSC3 tumors, and (e) substantially higher than gadoteridol in SCC4 tumors. Maximum CNR enhancement in subjects with MT218 was (f) borderline lower than gadoteridol in CAL27 tumors (p=0.0957), (g) borderline higher than gadoteridol in HSC3 tumors (p=0.0639), and (h) significantly higher than gadoteridol in SCC4 tumors. (f-h) Open dots connected with a solid line represent matched mouse-by-mouse data, and solid dots connected with a dotted line represent cohort averages. (i) Average maximum CNR enhancement was significantly different between the three tumor models in accordance with their EDB-FN expression with MT218, but not with gadoteridol. Data was analyzed with Two-Way ANOVA and Fisher's least significant difference post-hoc test (a-e,i) and unpaired T-tests (f-h) (*, p<0.05; **, p<0.01; ***, p<0.001; n.s., p>0.05).

FIG. 18 provides images showing the H&E staining of OSCC xenografts (left, n=3) showed various morphological features and stages of differentiation. Immunohistochemistry for EDB-FN with the EDB-FN-specific G4 antibody (right, n=3) showed weak, moderate and strong staining in the epithelial regions of CAL27, HSC3, and SCC4 tumors, respectively. Scale bars: 600 μm for H&E images, 200 μm for immunohistochemistry images.

FIGS. 19A-19C provide the structure (A), binding affinity measurement (B), and relaxivities (C) of the targeted contrast agent ZD2-N3-Gd(HP-DO3A) (MT218).

FIGS. 20A-20C provide fluorescence confocal microscopic images of extradomain B fibronectin expression taken with at 10× (Scale bar: 200 m) magnification in human pancreatic cancer (A), precancerous pancreatic intraepithelial neoplasia (PanIN) (B), and normal pancreatic tissues (C) stained with ZD2-Cy5.5 (red) and DAPI (blue). Binding of the ZD2-Cy5.5 due to EDB-FN expression reveals regions of malignancy and potential malignancy (arrowheads).

FIGS. 21A-21C provide Western blots of extradomain B fibronectin (EDB-FN) in BxPC3-GFP-Luc (BxPC3), Capan-1, and PANC-1-GFP-Luc (PANC-1) human PaCa cells with densiometric quantification (A), tumor xenografts derived from PaCa cells (B), and immunohistochemical staining of EDB-FN in PaCa xenografts with and without (NAC) G4 anti-EDB-FN monoclonal antibody (C, scale bar 200 m, 10× magnification). Western blots of normal tissues and hematoxylin & eosin (H&E) staining of the tumor specimens were also shown as references.

FIG. 22 provides representative T₁-weighted 2D spin-echo MR images of mice bearing Capan-1 flank xenografts before and at different time points after injection of MT218 (n=7) or Gd(HP-DO3A) (GTDL) (n=5) with the corresponding subtraction images (postcontrast subtracted by precontrast) overlaid on precontrast images. Regions with large changes in tumor signal of the subtraction images are indicated in white/red, regions with small changes in tumor signal are indicated in black/blue. Tumors are marked by white arrowheads, scale bar: 5 mm.

FIGS. 23A and 23B provide bioluminescence images of mice implanted with BxPC3-GFP-Luc intrapancreatic tumors (A). Representative T₁-weighted 2D spin-echo MR images of intrapancreatic tumors before and at different time points after injection of MT218 or Gd(HP-DO3A) (GTDL) at 0.1 mmol/kg and the corresponding subtraction image overlaid on precontrast images (n=3). Scale bar: 5 mm (B). Arrowheads point to the intrapancreatic BxPC3-GFP-Luc PaCa tumors.

FIGS. 24A and 24B provide bioluminescence images of the mice implanted with PANC-1-GFP-Luc intrapancreatic tumors (A) and representative T1-weighted 2D spin-echo MR images of the intrapancreatic tumors (n=6) before and 10 min after injection of MT218 or Gd(HP-DO3A) (GTDL) at 0.1 mmol/kg with subtraction images overlaid on pre-contrast images (B). Tumor locations are marked with arrowheads. Scale bar: 5 mm.

FIGS. 25A-25C provide contrast-to-noise ratios (CNR) of Capan-1 flank xenografts (A), BxPC-GFP-Luc intrapancreatic tumors (B) and liver calculated from MRI images at different time points before and after injection of MT218 or Gd(HP-DO3A) (gadoteridol). CNR of the PANC-1-GFP-Luc intrapancreatic PaCa tumors calculated from MRI images taken before and after injection of MT218 and Gd(HP-DO3A) (gadoteridol) (C) ** p<0.01; * p<0.05

FIGS. 26A-26C provide images showing the characterization of prostate cancer cells. Compared to low-risk LNCaP cells, LNCaP-CXCR2, C4-2, and C4-2-DR cells show A. increased invasion through Matrigel-coated transwell inserts, B. higher expression of EMT marker N-cadherin, and C. enhanced expression of EDB-FN in 3D cultures stained with EDB-FN-specific ZD2-Cy5.5 peptide probe.

FIGS. 27A & 27B provide graphs and images showing contrast-enhanced MRMI of EDB-FN with 0.04 mmol Gd/kg dose of MT218 facilitates non-invasive assessment of prostate cancer. T1-weighted 2D axial spin echo images were obtained before and 20 min post-injection of MT218 in athymic nu/nu mice. Compared to low risk LNCaP tumors, LNCaP-CXCR2, C4-2, and C4-2-DR xenografts show A. robust signal enhancement and B. higher CNRs.

FIGS. 28A-28D provide graphs and images showing contrast-enhanced MRMI of EDB-FN with 0.04 mmol Gd/kg dose of MT218 facilitates differential diagnosis of invasive drug-resistant prostate cancer. PC3-DR cells show A. decreased invasion through Matrigel-coated transwell inserts and B. lower expression of EDB-FN in 3D cultures stained with EDB-FN-specific ZD2-Cy5.5 peptide probe. T₁-weighted 2D axial spin echo images were obtained before and 20 min post-injection of MT218 in athymic nu/nu mice. Compared to less invasive PC3-DR tumors, the EDB-FN-rich invasive PC3 xenografts show C. robust signal enhancement and D. higher CNR.

FIGS. 29A-29D provide graphs and images showing contrast-enhanced MRMI of EDB-FN with 0.04 mmol Gd/kg dose of MT218 facilitates non-invasive active surveillance of prostate cancer. T1-weighted 2D axial spin echo images were obtained before and 20 min post-injection of MT218 in athymic nu/nu mice bearing C4-2 tumors. Progression of C4-2 tumors in two independent mice from day 21 to day 60 is accompanied by A-B. increase in signal enhancement and C-D. steady increase in CNRs.

FIGS. 30A-30D provide graphs and images showing the characterization of RGD-PEG-ECO/miR-200c nanoparticles. Size distribution and zeta potential of RGD-PEG-ECO/miR-200c and RGD-PEG-ECO/siNS nanoparticles as determined by dynamic light scattering (A); agarose gel retardation assay of RGD-PEG-ECO/miR-200c and RGD-PEG-ECO/siNS nanoparticles showing high encapsulation efficiency, when compared to free RNA controls (B); fluorescence confocal microscopic image showing efficient uptake of RGD-PEG-ECO/miR-200c-Cy5.5 nanoparticles in MDA-MB-231 cells transfected for 4 h (C), and the nanoparticle uptake quantified with FACS in comparison with untreated cells (D). Bars indicate mean±sem, *** p<0.005 using unpaired t-test.

FIGS. 31A-31P provide graphs and images showing RGD-PEG-ECO/miR-200c nanoparticles mediate robust and sustained delivery of miR-200c and downregulation of downstream target genes in TNBC cells. miR-200c upregulation for 14 days (A) and ZEB1 downregulation for 7 days (B) in MDA-MB-231 cells after transfection of RGD-PEG-ECO/miR-200c in comparison to RGD-PEG-ECO/siNS; the miR-200c levels in MDA-MB-231 (C) and Hs578T (I) at 48 h after transfection of RGD-PEG-ECO/miR-200c at 100 nM miR-200c and consequent downregulation of ZEB1 (D,J), BMI1 (E,K), survivin (F,L), FN1 (G,M), and EDB-FN (H,N) at the mRNA level in both cell lines as compared to RGD-PEG-ECO/siNS; western blots showing the downregulation of the downstream ZEB1, BMI1, Survivin, and EDB-FN proteins mediated by RGD-PEG-ECO/miR-200c as compared to RGD-PEG-ECO/siNS and non-specific control in MDA-MB-231 (O) and Hs578T (P) cells. Bars indicate mean±sem. * p<0.05, ** p<0.01, and *** p<0.005 using unpaired t-test.

FIGS. 32A-32C provide images showing RGD-PEG-ECO/miR-200c nanoparticles inhibit the migration, invasion, and spheroid formation of TNBC cells. The treatment of MDA-MB-231 and Hs578T cells with RGD-PEG-ECO/miR-200c inhibits their migration as shown by scratch-wound assay (A), invasion as shown by transwell assay (B), and tumor spheroid formation in 3D culture (C) when compared to RGD-PEG-ECO/siNS nanoparticles.

FIGS. 33A-33G provide graphs and images showing systemic administration of RGD-PEG-ECO/miR-200c nanoparticles significantly inhibited primary tumor progression in TNBC tumor xenografts. A single batch of RGD-PEG-ECO/siNS and RGD-PEG-ECO/miR-200c nanoparticles was formulated (N/P=8, 1.0 mg/kg RNA dose, 2.5 mol % MAL-PEG-RGD, 5% sucrose) and stored in −80° C. until use. A) Mice were treated according to the schematic of nanoparticle treatment schedule. Tumor growth was monitored weekly by BLI (B-E) and caliper measurements (F,G) in MDA-MB-231 and Hs578T tumor xenografts. Bars and lines indicate mean±sem. * p<0.05, ** p<0.01, and *** p<0.005 using unpaired t-test. Caliper measurements are compared to week 1.

FIGS. 34A-34D provide graphs and images showing MRMI of EDN-FN expression in the TNBC tumors treated with RGD-PEG-ECO/miR-200c. T1-weighted axial spin-echo images and CNR of the MDA-MB-231 (A,B) and Hs578T (C,D) tumors before and at different time points after intravenous injection of MT218. The MR images were acquired before and at the week 6 after weekly treatment RGD-PEG-ECO/miR-200c and RGD-PEG-ECO/siNS. Lines indicate mean±sem. * p<0.05, ** p<0.01, and *** p<0.005 using unpaired t-test.

FIGS. 35A-35G provide graphs and images showing systemic administration of RGD-PEG-ECO/miR-200c nanoparticles results in the upregulation of miR-200c and downregulation of EDB-FN. The levels of miR-200c and mRNA of ZEB1 and EDB-FN (A, B, C, D, E, F) and EDB-FN protein levels (G) in the MDA-MB-231 and Hs578T tumors treated with ECO/miR-200c and RGD-PEG-ECO/siNS were determined by qRT-PCR and immunohistochemistry, where the tumors were dissected at the week 7.

FIGS. 36A-36D provide graphs and images showing systemic administration of RGD-PEG-ECO/miR-200c nanoparticles does not induce evident adverse side-effects in the treated mice. A) Both RGD-PEG-ECO/miR-200c- and RGD-PEG-ECO/siNS-treated mice showed no significant differences in body weights during the 6 weeks of treatment. The assessment of histology of liver (B), spleen (C), and kidney (D) by H&E staining showed no overt tissue damage. (Scale bar=100 μm)

### DETAILED DESCRIPTION

The present invention provides a method of detecting drug-resistant cancer in a subject. The method includes contacting a tissue of the subject with an effective amount of a molecular probe, detecting the amount of the molecular probe present in the tissue, comparing the amount of molecular probe detected to a control value, and detecting drug-resistant cancer in the subject if the amount of the molecular probe present in the tissue is higher than the control value. The molecular probe includes the following formula: P-L-C wherein P is an EDB-FN targeting peptide, C is a contrast agent; and L is a non-peptide linker that covalently links the peptide to the contrast agent. Methods of monitoring the treatment of drug resistant cancer are also provided.

Unless otherwise defined, all technical and scientific terms used herein have the same meaning as commonly understood by one of ordinary skill in the art to which these inventions belong. The terminology used in the description of the inventions herein is for describing particular embodiments only and is not intended to be limiting of the inventions. As used in the description of the inventions and the appended claims, the singular forms “a,” “an,” and “the” are intended to include the plural forms as well, unless the context clearly indicates otherwise.

The numerical ranges and parameters setting forth the broad scope of the inventions are in some cases approximations. Nonetheless, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, the term “diagnosis” can encompass determining the nature of disease in a subject, as well as determining the severity and probable outcome of disease or episode of disease and/or prospect of recovery (prognosis). “Diagnosis” can also encompass diagnosis in the context of rational therapy, in which the diagnosis guides therapy, including initial selection of therapy, modification of therapy (e.g., adjustment of dose and/or dosage regimen), and the like.

Treat”, “treating”, and “treatment”, etc., as used herein, refer to any action providing a benefit to a subject at risk for or afflicted with a condition or disease such as cancer, including improvement in the condition through lessening or suppression of at least one symptom, delay in progression of the disease, prevention or delay in the onset of the disease, etc. The subject may be at risk due to exposure to carcinogenic agents, being genetically predisposed to disorders characterized by unwanted, rapid cell proliferation, and so on.

The terms “subject,” and “patient” are used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. Diagnosis of humans is of particular interest.

As used herein, the term “organic group” is used to mean a hydrocarbon group that is classified as an aliphatic group, cyclic group, or combination of aliphatic and cyclic groups (e.g., alkaryl and aralkyl groups). In the context of the present invention, suitable organic groups for the compounds of this invention are those that do not interfere with the anti-cancer activity of the compounds. In the context of the present invention, the term “aliphatic group” means a saturated or unsaturated linear or branched hydrocarbon group. This term is used to encompass alkyl, alkenyl, and alkynyl groups, for example.

As used herein, the terms “alkyl”, “alkenyl”, and the prefix “alk-” are inclusive of straight chain groups and branched chain groups. Unless otherwise specified, these groups contain from 1 to 20 carbon atoms, with alkenyl groups containing from 2 to 20 carbon atoms.

In some embodiments, these groups have a total of at most 10 carbon atoms, at most 8 carbon atoms, at most 6 carbon atoms, or at most 4 carbon atoms. Alkyl groups including 4 or fewer carbon atoms can also be referred to as lower alkyl groups. Alkyl groups can also be referred to by the number of carbon atoms that they include (i.e., C₁-C₄ alkyl groups are alky groups including 1-4 carbon atoms).

When a group is present more than once in any formula or scheme described herein, each group (or substituent) is independently selected, whether explicitly stated or not. For example, for the formula —C(O)—NR₂ each R group is independently selected.

As used herein, the term “polypeptide” is intended to encompass a singular “polypeptide” as well as plural “polypeptides,” and comprises any chain or chains of two or more amino acids. Thus, as used herein, terms including, but not limited to “peptide,” “dipeptide,” “tripeptide,” “protein,” “amino acid chain,” or any other term used to refer to a chain or chains of two or more amino acids, are included in the definition of a “polypeptide,” and the term “polypeptide” may be used instead of, or interchangeably with any of these terms.

The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

“Amino acid” is used herein to refer to a chemical compound with the general formula: NH₂—CRH—COOH, where R, the side chain, is H or an organic group. Where R is organic, R can vary and is either polar or nonpolar (i.e., hydrophobic). The following abbreviations are used throughout the application: A=Ala=Alanine, T=Thr=Threonine, V=Val=Valine, C=Cys=Cysteine, L=Leu=Leucine, Y=Tyr=Tyrosine, I=Ile=Isoleucine, N=Asn=Asparagine, P=Pro=Proline, Q=Gln=Glutamine, F=Phe=Phenylalanine, D=Asp=Aspartic Acid, W=Trp=Tryptophan, E=Glu=Glutamic Acid, M=Met=Methionine, K=Lys=Lysine, G=Gly=Glycine, R=Arg=Arginine, S=Ser=Serine, H=His=Histidine. Unless otherwise indicated, the term “amino acid” as used herein also includes amino acid derivatives that nonetheless retain the general formula.

### Methods of Detecting Drug-Resistant Cancer

In one aspect, the present invention provides a method of detecting drug-resistant cancer in a subject. The method includes the steps of contacting a tissue of the subject with an effective amount of a molecular probe, detecting the amount of the molecular probe present in the tissue, comparing the amount of molecular probe detected to a control value; and detecting drug-resistant cancer in the subject if the amount of the molecular probe present in the tissue is higher than the control value.

The molecular probes can be used in a method to detect and/or determine the presence, location, and/or distribution of drug-resistant cancer cells expressing EDB-FN in an organ, tissue, or body area of a subject. The presence, location, and/or distribution of the molecular probe in the animal's tissue, e.g., prostate tissue, can be visualized (e.g., with an in vivo imaging modality described herein). “Distribution” as used herein is the spatial property of being scattered about over an area or volume. In this case, “the distribution of cancer cells” is the spatial property of cancer cells being scattered about over an area or volume included in the subject's tissue, e.g., prostate tissue. The distribution of the molecular probe may then be correlated with the presence or absence of drug-resistant cancer cells in the tissue.

In one aspect, the molecular probes may be administered to a subject to assess the distribution of drug-resistant cancer cells in a subject and correlate the distribution to a specific location. Surgeons routinely use stereotactic techniques and intra-operative MRI (iMRI) in surgical resections. This allows them to specifically identify and sample tissue from distinct regions of the tumor such as the tumor edge or tumor center. Frequently, they also sample regions of tissue on the tumor margin that are outside the tumor edge that appear to be grossly normal but are infiltrated by dispersing tumor cells upon histological examination.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancer cell” refers to a cell undergoing early, intermediate or advanced stages of multi-step neoplastic progression. The features of early, intermediate and advanced stages of neoplastic progression have been described using microscopy. Cancer cells at each of the three stages of neoplastic progression generally have abnormal karyotypes, including translocations, inversion, deletions, isochromosomes, monosomies, and extra chromosomes. Cancer cells include “hyperplastic cells,” that is, cells in the early stages of malignant progression, “dysplastic cells,” that is, cells in the intermediate stages of neoplastic progression, and “neoplastic cells,” that is, cells in the advanced stages of neoplastic progression. Examples of cancers are sarcoma, breast, lung, brain, bone, liver, kidney, colon, ovarian, and prostate cancer. In some embodiments, the cancer is breast cancer, oral cancer, pancreatic cancer, or prostate cancer. A tumor is the physical manifestation of cancer within a subject.

The present invention provides methods of detecting and monitoring treatment of drug-resistant cancer. Drug-resistant cancer is a type of cancer that has developed resistance to treatment with anticancer agents. The various facets of drug resistance are known to those skilled in the art. See Vasan et al., Nature volume 575, 299-309 (2019). Drug-resistant cancer can include intrinsic and acquired resistance. Examples of factors contributing to drug resistance include tumor burden, tumor heterogeneity, the generation or use of physical barriers by the tumor, immunosuppression, and undruggable genomic drivers.

The method includes the step of contacting a tissue of the subject. Contacting, as used herein, refers to causing two items to become physically adjacent and in contact, or placing them in an environment where such contact will occur within a reasonably short timeframe. For example, contacting a tissue with a molecular probe includes directly applying the molecular probe to a tissue, such as a biopsy sample that has been obtained from a subject. Contacting can include contacting in vivo, ex vivo, and in vitro. However, contacting also includes systemic administration which results in contact between the molecular probe and the tissue through circulation-mediated contact. Accordingly, in some embodiments, the tissue is contacted in vivo.

In some embodiments, the molecular probe is systemically administered to a subject having or suspected of having cancer. General signs and symptoms associated with cancer include fatigue, wight changes, or a lump or area of thickening that can be felt under the skin. Most cancer signs and symptoms are specific to the tissue in which cancer has occurred. For example, headaches or seizures can be a sign of brain cancer, while trouble urinating can be a sign of bladder cancer. Nonetheless, the various signs and symptoms of cancer are well-known to those skilled in the art, and are described on the National Cancer Institute Website. Symptoms of cancer can indicate that a subject has or is suspected of having cancer, while other risk factors such as genetic predisposition and exposure to radiation can also lead a subject to being suspected of having cancer.

A tissue region is an area of tissue in the subject which is being treated and/or analyzed. Generally, the tissue region is within the tissue where cancer has been identified, or tissues where it is suspected that cancer may have spread through metastasis. The tissue region can be an organ of a subject such as the heart, lungs, or blood vessels. In other embodiments, the tissue region can be diseased tissue, or tissue that is suspected of being diseased, such as a tumor or tissue regions connected with the tumor by a metastatic route or a tissue having similar characteristics to the primary tumor tissue. Examples of metastatic routes include the transcoelomic route (penetration of the surface of the peritoneal, pleural, pericardial, or subarachnoid space), lymphatic route (transport of tumor cells to lymph nodes and from there to other parts of the body), and the haematogenous route (used by sarcomas and carcinomas). The tissue region can vary widely in size, and can for example range from a size of about 1 cm³ to about 500 cm³.

The molecular probe can be detected using a variety of different imaging techniques, depending on the imaging group included in the molecular probe. Examples of imaging methods include gamma imaging, positron emission tomography (PET) imaging, computer tomography (CT) imaging, magnetic resonance imaging (MRI), near infrared imaging, and fluorescent imaging.

In some embodiments, the molecular probe includes an imaging group suitable for use as a magnetic resonance imaging agent. Disease detection using MRI is often difficult because areas of disease have similar signal intensity compared to surrounding healthy tissue. In the case of magnetic resonance imaging, the imaging agent can also be referred to as a contrast agent. Lanthanide elements are known to be useful as contrast agents. The lanthanide chemical elements comprise the fifteen metallic chemical elements with atomic numbers 57 through 71, and include lanthanum, cerium, praseodymium, neodymium, promethium, samarium, europium, gadolinium, terbium, dysprosium, holmium, erbium, thulium, ytterbium, and lutetium. Preferred lanthanides include europium, gadolinium, and terbium. In order to more readily handle these rare earth metals, the lanthanides are preferably chelated. In some embodiments, the lanthanide selected for use as an imaging group is gadolinium, or more specifically gadolinium (III).

### Molecular Probes

The present invention includes administering and detecting molecular probes. The molecular probes referred to herein generally comprise the formula:

P-L-C,

wherein:
P is a peptide that includes an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9;
C is a contrast agent; and
L is a non-peptide linker that covalently links the peptide to the contrast agent, the linker including a carboxylic acid that forms a carboxamide with an amine of the peptide or a malemide that forms a thioester bond with a cysteine reside of the peptide or a maleimide that forms a thioester with a cysteine residue of the peptide.

The molecular probes described herein include targeting peptides with a peptide sequence that specifically binds to extradomain-B fibronectin (EDB-FN). Cancer, and particularly drug-resistant cancer has a unique tumor microenvironment that facilitates cancer cell survival, proliferation, and metastasis. High expression of EDB-FN is correlated with the presence of drug-resistant cancer.

Molecular probes including the targeting peptides can be administered systemically to a subject, such as by intravenous or parenteral administration, and readily target the extracellular matrix proteins EDB-FN to define cancer cell location, distribution, and/or aggressiveness as well as tumor cell margins in the subject. For a description of the molecular probes, and how they can be prepared, see U.S. Pat. No. 10,925,980, the disclosure of which is incorporated herein by reference in its entirety.

In some embodiments, the molecular probe can include the following formula:

P-L-C

wherein P is a targeting peptide; C is a contrast agent; and L is a non-peptide linker that covalently links the peptide to the contrast agent. The linker can include a carboxylic acid that forms a carboxamide with an amine of the peptide or a maleimide that forms a thioester bond with a cysteine residue of the peptide.

In some embodiments, the targeting peptide can specifically bind to EDB-FN. Targeting peptides that specifically bind EDB-FN can include linear peptides having the amino acid sequences of TVRTSAD (SEQ ID NO: 1), NWGDRIL (SEQ ID NO: 2), NWGKPIK (SEQ ID NO: 3), SGVKSAF (SEQ ID NO: 4), GVKSYNE (SEQ ID NO: 5), IGKTNTL (SEQ ID NO: 6), IGNSNTL (SEQ ID NO: 7), IGNTIPV (SEQ ID NO: 8), and LYANSPF (SEQ ID NO: 9), cyclic peptides having the amino acid sequences of CTVRTSADC (SEQ ID NO: 10), CNWGDRILC (SEQ ID NO: 11), CNWGKPIKC (SEQ ID NO: 12), CSGVKSAFC (SEQ ID NO: 13), CGVKSYNEC (SEQ ID NO: 14), CIGKTNTLC (SEQ ID NO: 15), CIGNSNTLC (SEQ ID NO: 16), CIGNTIPVC (SEQ ID NO: 17), or CLYANSPFC (SEQ ID NO: 18), or linear peptides with cysteine linkers CTVRTSAD (SEQ ID NO: 31), CNWGDRIL (SEQ ID NO: 32), CNWGKPIK (SEQ ID NO: 33), CSGVKSAF (SEQ ID NO: 34), CGVKSYNE (SEQ ID NO: 35), CIGKTNTL (SEQ ID NO: 36), CIGNSNTL (SEQ ID NO: 37), CIGNTIPV (SEQ ID NO: 38), and CLYANSPF (SEQ ID NO: 39). In other embodiments, the targeting peptide can specifically bind to EDA-FN. Targeting peptides that specifically bind EDA-FN can include linear peptides having the amino acid sequences of WNYPFRL (SEQ ID NO: 19), SNTSYVN (SEQ ID NO: 20), SFSYTSG (SEQ ID NO: 21), WSPAPMS (SEQ ID NO: 22), TREHPAQ (SEQ ID NO: 23), or ARIIDNA (SEQ ID NO: 24), cyclic peptides having the amino acid sequences of CWNYPFRLC (SEQ ID NO: 25), CSNTSYVNC (SEQ ID NO: 26), CSFSYTSGC (SEQ ID NO: 27), CWSPAPMSC (SEQ ID NO: 28), CTREHPAQC (SEQ ID NO: 29), or CARIIDNAC (SEQ ID NO: 30), or linear peptides with cysteine linkers CTVRTSAD (SEQ ID NO: 40), CNWGDRIL (SEQ ID NO: 41), CNWGKPIK (SEQ ID NO: 42), CSGVKSAF (SEQ ID NO: 43), CGVKSYNE (SEQ ID NO: 44), CIGKTNTL (SEQ ID NO: 45), CIGNSNTL (SEQ ID NO: 46), CIGNTIPV (SEQ ID NO: 47), and CLYANSPF (SEQ ID NO: 48).

The targeting peptides can be subject to various changes, substitutions, insertions, and deletions where such changes provide for certain advantages in its use. In this regard, targeting peptides that bind to and/or complex with EDB-FN can be substantially homologous with, rather than be identical to, the sequence of a recited peptide where one or more changes are made and it retains the ability to function as specifically binding to and/or complexing with EDB-FN.

The targeting peptides can be in any of a variety of forms of polypeptide derivatives, that include amides, conjugates with proteins, cyclized polypeptides, polymerized polypeptides, analogs, fragments, chemically modified polypeptides, and the like derivatives.

The term “analog” includes any peptide having an amino acid residue sequence substantially identical to a sequence specifically shown herein in which one or more residues have been conservatively substituted with a functionally similar residue and that specifically binds to and/or complexes with EDB-FN as described herein. Examples of conservative substitutions include the substitution of one non-polar (hydrophobic) residue, such as isoleucine, valine, leucine or methionine for another, the substitution of one polar (hydrophilic) residue for another, such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such peptide displays the requisite binding activity.

“Chemical derivative” refers to a subject peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-benzylhistidine. Also included as chemical derivatives are those polypeptides, which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine.

Peptides described herein also include any peptide having one or more additions and/or deletions or residues relative to the sequence of a peptide whose sequence is shown herein, so long as the requisite binding specificity or activity is maintained.

The term “fragment” refers to any subject peptide having an amino acid residue sequence shorter than that of a polypeptide whose amino acid residue sequence is shown herein.

Additional residues may also be added at either terminus of a peptide for the purpose of providing a “linker” by which the peptides can be conveniently linked and/or affixed to other polypeptides, proteins, detectable moieties, labels, solid matrices, or carriers.

Amino acid residue linkers are usually at least one residue and can be 40 or more residues, more often 1 to 10 residues. Typical amino acid residues used for linking are glycine, tyrosine, cysteine, lysine, glutamic and aspartic acid, or the like. In addition, a subject targeting peptide agent can differ by the sequence being modified by terminal-NH₂ acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half-life of the polypeptides in solutions, particularly biological fluids where proteases may be present. In this regard, polypeptide cyclization is also a useful terminal modification, and is particularly preferred also because of the stable structures formed by cyclization and in view of the biological activities observed for such cyclic peptides as described herein.

In some embodiments, the non-peptide linker is a non-peptide aliphatic or heteroaliphatic linker. The non-peptide linker can include an alkylene dicarboxamide that covalently links the peptide and contrast agent.

In some embodiments, the non-peptide linker can include a first portion that is about 1 to about 10 atoms in lengths and second portion that acts as a spacer. The portion of the linker that acts a spacer can include a non-peptide polymer that includes but is not limited to a polyalkyleneoxide, polyvinyl alcohol, polyethylene glycol (PEG), polypropylene glycol (PPG), co-poly (ethylene/propylene) glycol, polyoxyethylene (POE), polyurethane, polyphosphazene, polysaccharides, dextran, polyvinylpyrrolidones, polyvinyl ethyl ether, polyacryl amide, polyacrylate, polycyanoacrylates, lipid polymers, chitins, hyaluronic acid, and heparin. For more detailed descriptions of spacers for non-peptide linkers, see, for example, WO/2006/107124, which is incorporated by reference herein. Typically such linkers will have a range of molecular weight of from about 1 kDa to 50 kDa, depending upon a particular linker. For example, a typical PEG has a molecular weight of about 1 to 5 kDa, and polyethylene glycol has a molecular weight of about 5 kDa to 50 kDa, and more preferably about 10 kDa to 40 kDa.

The contrast agent is directly conjugated to the targeting peptide with the linker. The role of the contrast agent is to facilitate the detection step of a detection or diagnostic method by allowing visualization of the complex formed by binding of a molecular probe comprising a targeting peptide to EDB-FN. The contrast agent can be selected such that it generates a signal, which can be measured and whose intensity is related (preferably proportional) to the amount of the molecular probe bound to the tissue being analyzed.

In certain embodiments, the contrast agent includes a chelating agent and a metal ion. The chelating agent generally possesses one or more groups capable of forming a covalent bond with the linker. A number of different chelating agents known in the art can be used herein. In one aspect, the chelating agent comprises an acyclic or cyclic compound comprising at least one heteroatom (e.g., oxygen, nitrogen, sulfur, phosphorous) that has lone-pair electrons capable of coordinating with the imaging agent. An example of an acyclic chelating agent includes ethylenediamine Examples of cyclic chelating agents include diethylenetriaminepentaacetate (DTPA) or its derivatives, 1,4,7,10-tetraazadodecanetetraacetate (DOTA) and its derivatives, 1,4,7,10-tetraazadodecane-1,4,7-triacetate (DO3A) and its derivatives (e.g., HP-DO3A), ethylenediaminetetraacetate (EDTA) and its derivatives, 1,4,7,10-tetraazacyclotridecanetetraacetic acid (TRITA) and its derivatives, 1,4,8,11-tetraazacyclotetradecane-1,4,8,11-tetraacetic acid (TETA) and its derivatives, 1,4,7,10-tetraazadodecanetetramethylacetate (DOTMA) and its derivatives, 1,4,7,10-tetraazadodecane-1,4,7-trimethylacetate (DO3MA) and its derivatives, N,N′,N″,N′″-tetraphosphonatomethyl-1,4,7,10-tetraazacyclododecane (DOTP) and its derivatives, 1,4,7,10-tetraazacyclododecane-1,4,7,10-tetrakis(methylene methylphosphonic acid) (DOTMP) and its derivatives, 1,4,7,10-tetraazacyclododecane-1,4,7,10-tetrakis(methylene phenylphosphonic acid) (DOTPP) and its derivatives, or N,N′-ethylenedi-L-cysteine or its derivatives. The term “derivative” is defined herein as the corresponding salt and ester thereof of the chelating agent.

The selection of the metal ion can vary depending upon the detection technique (e.g., MRI, PET, etc.). Metal ions useful in magnetic resonance imaging can include Gd⁺³, Eu⁺³, Tm⁺³, Dy⁺³, Yb⁺³, Mn⁺², Fe⁺³, ⁵⁵Co, ⁶⁴Cu, ⁶⁷Cu, ⁴⁷Sc, ⁶⁶Ga ⁶⁸Ga, ⁹⁰Y, ⁹⁷Ru, ⁹⁹mTc, ¹¹¹h, ¹⁰⁹Pd, ¹⁵³Sm, ¹⁷⁷Lu, ¹⁸⁶Re, and ¹⁸⁸Re. In some embodiments, the contrast agent of the molecular probe includes at least one of metal chelating agent or a metallofullerene. In some embodiments, the contrast agent can include a metallofullerene, such as Gd3N@C80.

In some embodiments, the molecular probe can have the formula:

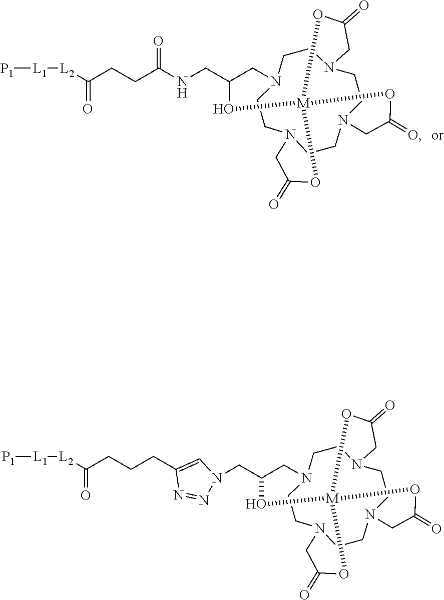

wherein:
P₁ is a peptide including an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9;
L₁ is an optional spacer,
L₂ is an amino group of the peptide P₁ or the spacer, and
M is a metal selected from the group consisting of Gd⁺³, Eu⁺³, Tm⁺³, Dy⁺³, Yb⁺³, Mn⁺², Fe⁺³, ⁵⁵Co, ⁶⁴Cu, ⁶⁷Cu, ⁴⁷Sc, ⁶⁶Ga ⁶⁸Ga, ⁹⁰y ⁹⁷Ru, ⁹⁹mTc, ¹¹¹h, ¹⁰⁹Pd, ¹⁵3Sm, ¹⁷⁷Lu, ¹⁸⁶Re, and ¹⁸⁸Re;
or salts thereof.

In some embodiments, L₁ can include at least one of a polyalkyleneoxide, polyvinyl alcohol, polyethylene glycol (PEG), polypropylene glycol (PPG), co-poly (ethylene/propylene) glycol, polyoxyethylene (POE), polyurethane, polyphosphazene, polysaccharides, dextran, polyvinylpyrrolidones, polyvinyl ethyl ether, polyacryl amide, polyacrylate, or polycyanoacrylates.

In other embodiments, the probe can have the formula:

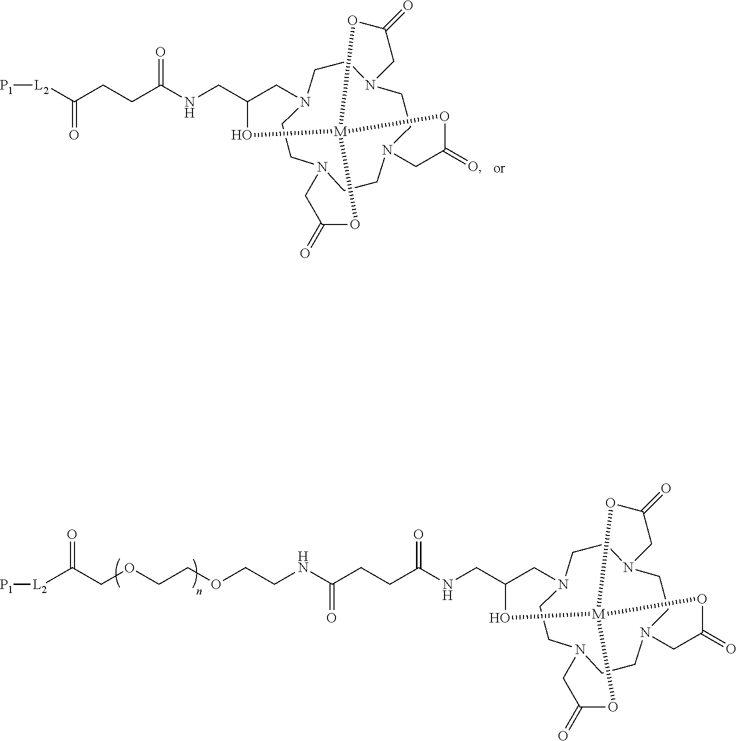

wherein:
P₁ is a peptide having an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9;
L₂ is an amino group of the peptide P₁, and
M is a metal selected from the group consisting of Gd⁺³, Eu⁺³, Tm⁺³, Dy⁺³, Yb⁺³, Mn⁺², Fe⁺³, ⁵⁵Co, ⁶⁴Cu, ⁶⁷Cu, ⁴⁷Sc, ⁶⁶Ga ⁶⁸Ga, ⁹⁰Y, ⁹⁷Ru, ⁹⁹mTc, ¹¹¹h, ¹⁰⁹Pd, ¹⁵³Sm, ¹⁷⁷Lu, ¹⁸⁶Re, and ¹⁸⁸Re, and n is an integer from 0 to 100; or salts thereof.

In some embodiments, to identify and facilitate removal of cancers cells, microscopic intra-operative imaging (IOI) techniques can be combined with systemically administered or locally administered molecular probes described herein. The molecular probe upon administration to the subject can target and detect and/or determine the presence, location, and/or distribution of drug-resistant cancer cells, i.e., cancer cells associated with EDB-FN expression, in an organ or body area of a patient. In one example, the molecular probe can be combined with IOI to identify malignant cells that have infiltrated and/or are beginning to infiltrate at a tumor margin. The method can be performed in real-time during surgery. The method can include local or systemic application of the molecular probe that includes a detectable moiety, such as a PET, fluorescent, or MRI contrast moiety. An imaging modality can then be used to detect and subsequently gather image data. The resultant image data may be used to determine, at least in part, a surgical and/or radiological treatment. Alternatively, this image data may be used to control, at least in part, an automated surgical device (e.g., laser, scalpel, micromachine) or to aid in manual guidance of surgery. Further, the image data may be used to plan and/or control the delivery of a therapeutic agent (e.g., by a micro-electronic machine or micro-machine).

### Methods of Monitoring Treatment of Drug-Resistant Cancer

A method of monitoring the treatment of drug resistant cancer, comprising: contacting a tissue of a subject undergoing treatment of drug resistant cancer with an effective amount of a molecular probe, detecting a first amount of the molecular probe present in the tissue, contacting the tissue of the subject for a second time with an effective amount of a molecular probe comprising the formula P-L-C, detecting a second amount of the molecular probe present in the tissue, and comparing the first amount and the second amount of the molecular probe to monitor the treatment of the drug resistant cancer in the subject.

In certain embodiments, the methods and molecular probes described herein can be used to measure the efficacy of treatment of drug-resistant cancer. In this embodiment, the molecular probe can be administered to the subject prior to, during, or post treatment and the distribution of cancer cells can be imaged to determine the efficacy of the treatment. In one example, the treatment can include a surgical resection of the metastatic cancer and the molecular probe can be used to define the distribution of the metastatic cancer pre-operative and post-operative to determine the efficacy of the surgical resection. Optionally, the methods and molecular probes can be used in an intra-operative surgical procedure, such as a surgical tumor resection, to more readily define and/or image the cancer cell mass or volume during the surgery.

Treatment can also include administration of a cancer therapeutic or other types of cancer therapy. A “cancer therapeutic,” as used herein, can include any agent that is capable of negatively affecting cancer in an animal, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of an animal with cancer. Cancer therapy can include such as, but not limited to, chemotherapies, radiation therapies, hormonal therapies, and/or biological therapies/immunotherapies. A reduction, for example, in cancer volume, growth, migration, and/or dispersal in a subject may be indicative of the efficacy of a given therapy. This can provide a direct clinical efficacy endpoint measure of a cancer therapeutic. Therefore, in another aspect, a method of monitoring the efficacy of a cancer therapeutic is provided. More specifically, embodiments of the application provide for a method of monitoring the efficacy of a cancer therapy.

The therapeutic agent can include an anti-proliferative agent that exerts an antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agent agents available in commercial use, in clinical evaluation and in preclinical development. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

Special methods can be used to treat drug-resistant cancer. One method of treating drug-resistant cancer is to use combination therapy in which a plurality of chemotherapeutic agents having non-overlapping mechanisms of action are used. See Bosl et al., N. Eng. J. Med., 294, 405-410 (1986). Drug-resistant cancer can also be treated by varying dose intensity or using high doses of chemotherapy. See Sternberg et al., J. Clin. Oncol., 19, 2638-2646 (2001). Other methods include the use of therapies specifically targeted to enabling characteristics of the drug-resistant cancer, such as targeting tyrosine kinase, nuclear receptors, or estrogen receptors. See Hanahan D., Weinberg R. A., Cell, 144, 646-674 (2011). Immunological approaches, and in particular the use of monoclonal antibodies to disable immune checkpoints, can also be used to treat drug resistant cancer. See Ribas A., Wolchok J. D., Science, 359, 1350-1355 (2018).

In certain embodiments, the methods and molecular probes described herein can be used to monitor the treatment of a subject having drug-resistant cancer. In this embodiment, the tissue of the subject is contacted with an effective amount of a molecular probe a first time prior to, during, or post administration of the therapeutic regimen and the amount and/or distribution of cancer cells can be imaged to determine the efficacy of the treatment. Then, at a later time, the tissue of the subject is contacted with an effective amount of the molecular probe for a second time, and a second amount and/or distribution of the molecular probe present in the tissue is detected. The first amount and the second amount of the molecular probe (and/or its distribution) are then compared to monitor the treatment of the drug resistant cancer in the subject.

### Administration and Formulation

The molecular probe described herein can be administered to the subject by, for example, systemic, topical, and/or parenteral methods of administration. These methods include, e.g., injection, infusion, deposition, implantation, or topical administration, or any other method of administration where access to the tissue by the molecular probe is desired. In one example, administration of the molecular probe can be by intravenous injection of the molecular probe in the subject. Single or multiple administrations of the probe can be given. “Administered”, as used herein, means provision or delivery of a molecular probe in an amount(s) and for a period of time(s) effective to label cancer cells in the subject.

Molecular probes comprising the targeting peptides described herein can be administered to a subject in an effective amount of a pharmaceutical composition containing a molecular probe or a pharmaceutically acceptable water-soluble salt thereof, to a patient.

An “effective amount” means that the amount of the molecular probe that is administered is sufficient to enable detection of binding or complexing of the probe to EDB-FN and/or EDA-FN expressed by the cancer cells or other cells in the cancer cell microenvironment. An “imaging effective quantity” means that the amount of the molecular probe that is administered is sufficient to enable imaging of binding or complexing of the molecular probe to the EDB-FN and/or EDA-FN of the cancer cells or other cells in the cancer cell microenvironment.

Formulation of the molecular probe to be administered will vary according to the route of administration selected (e.g., solution, emulsion, capsule, and the like). Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like.

The preparation of a pharmacological composition that contains active ingredients dissolved or dispersed therein is well understood in the art. Typically such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

Any polypeptide or compound may also be used in the form of a pharmaceutically acceptable salt. Acids, which are capable of forming salts with the polypeptides, include inorganic acids such as trifluoroacetic acid (TFA) hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like.

Bases capable of forming salts with the polypeptides include inorganic bases, such as sodium hydroxide, ammonium hydroxide, potassium hydroxide and the like; and organic bases such as mono-, di- and tri-alkyl and aryl-amines (e.g., triethylamine, diisopropylamine, methylamine, dimethylamine and the like) and optionally substituted ethanolamines (e.g., ethanolamine, diethanolamine and the like).

The following examples are included for purposes of illustration and is not intended to limit the scope of the invention.

### EXAMPLES

### Example 1: Non-Invasive Assessment and Therapeutic Monitoring of Drug-Resistant Colorectal Cancer by MR Molecular Imaging of Extradomain-B Fibronectin

Colorectal cancer (CRC) is the second most common cause of cancer death in men and women combined in the US. The 5-year survival for CRC cases in 2013 decreased from 88.1% at stage I to 12.6% at stage IV of diagnosis. Multiple studies and clinical trials have proven significantly reduced CRC mortality from preventive screening. Although highly treatable with chemotherapy and surgery in early stages, 80-90% metastatic CRC is inoperable at diagnosis and requires neoadjuvant chemotherapy. Furthermore, development of multidrug resistance can also lead to relapse and distant metastases, which are incurable. Current standards for CRC treatment monitoring follow the RECIST (Response Evaluation Criteria in Solid Tumors) guidelines (Eisenhauer, E. A., et al., Eur J Cancer, 2009. 45(2): p. 228-47), which are limited due to their reliance on tumor anatomical size, lesion irregularities leading to subjective opinions, and delays in detection of negative tumor response to therapies. To improve the outcomes of chemo- or targeted therapies and to enable decision making for adaptive interventions, non-invasive and repeated imaging for accurate detection and surveillance of invasive, drug-resistant tumor populations is imperative.

Clinical CRC diagnostics utilizes blood markers like carcinoembryonic antigen (CEA) and guaiac-based fecal occult blood test (GOBT), which suffer from low sensitivity and specificity. Liquid biopsies that measure mutational burden from circulating tumor DNA (ctDNA) are reflective of inter-patient and inter- and intra-tumor heterogeneity, and are fast gaining momentum in the diagnostic and prognostic arenas; however, they cannot provide spatial information on neoplastic lesions, rendering diagnostic imaging indispensable. A common imaging modality for CRC treatment monitoring is positron emission tomography-computed tomography (PET-CT), usually with the [¹⁸F]-fluoro-2-deoxyglucose (¹⁸F-FDG) radiotracer, which provides functional and metabolic data, but is limited by radiation exposure and confounding factors like cell density, hyperglycemia, and poor resolution. Contrast-enhanced magnetic resonance imaging (MRI) employs Gd(III)-based contrast agents (GBCAs) that shorten T₁ of tissues, and T₁-weighted imaging is also routinely used for diagnosis and surveillance of CRC. Jhaveri, K. S. and H. Hosseini-Nik, AJR Am J Roentgenol, 2015. 205(1): p. W42-55. This current clinical assessment is fraught with serious challenges in that, clinical non-targeted GBCAs are non-specific and unable to distinguish aggressive drug-resistant tumor species from the sensitive ones; and repeated administration of GBCAs is associated with potential safety concerns of Gd-based toxicity and brain deposition. Given the extensive molecular and phenotypic plasticity of CRC tumors, it is essential to develop molecular imaging agents and strategies that can accurately identify the trajectory of CRC during oncotherapy, based on alterations in key biologically relevant microenvironmental characteristics of individual tumors and tumor niches.

To date, the discipline of molecular cancer imaging has witnessed a limited number of studies, possibly due to the dearth of reliable biomarkers. Although colon cancer-secreted protein 2 (CCSP-2) was used as a molecular marker for near-infrared fluorescence imaging of primary tumors, patient-derived xenografts, and liver metastases, this marker is only specific to colon adenomas. Among the widely reported magnetic nanoparticle (MNP) studies, MRI-based detection of iron oxide nanoparticles targeting human tumor antigen underglycosylated mucin 1 (uMUC1), and nanoaggregation of probe scaffolds specific to pro-apoptotic caspases, has been used to monitor chemotherapeutic response of colon xenografts. Zhou, Z., et al., Adv Mater, 2019. 31(8): p. e1804567. Vascular volume fraction (VVF), K-Ras mutation, and EGFR, have also been used as surrogate markers for MRI of CRC with limited success. However, MR molecular imaging (MRMI) for non-invasive assessment and treatment monitoring of drug-resistant CRC has never been done before. To address this challenge, to circumvent the immune and translational hurdles of MNPs, and to potentially target a broader range of drug-resistant cancers, we developed MRMI strategies targeted to the abundantly expressed oncoprotein extradomain-B fibronectin (EDB-FN) in the tumor extracellular matrix (ECM). Han, Z. and Z. R. Lu, J Mater Chem B, 2017. 5(4): p. 639-654; Lu, Z. R., Curr Opin Biomed Eng, 2017. 3: p. 67-73.

EDB-FN, an oncofetal isoform of fibronectin, is overexpressed in a multitude of cancers. Han, Z., et al., Bioconjug Chem, 2017. 28(4): p. 1031-1040; Han, Z., et al., Nat Commun, 2017. 8(1): p. 692. EDB-FN is also elevated in CRC and is associated with angiogenesis, growth and tissue remodeling. Santimaria, M., et al., Clin Cancer Res, 2003. 9(2): p. 571-9. Literature studies show that even within the same cancer type, EDB-FN is preferentially upregulated in the more invasive cell and tumor subtypes, compared to the indolent ones. Han, Z., et al., Magn Reson Med, 2018. 79(6): p. 3135-3143. This specific spatial and temporal expression of EDB-FN in malignancies and its absence in healthy tissues renders it an attractive target for molecular imaging and targeted therapy. Han, Z., et al., Bioconjug Chem, 2019. 30(5): p. 1425-1433. We previously designed and developed an EDB-FN-targeted GBCA, ZD2-N₃—Gd(HP-DO3A) (MT218), by conjugating the EDB-FN-specific peptide ZD2 to the clinical GBCA Gadoteridol (Han, Z., et al., Bioconjug Chem, 2015. 26(5): p. 830-8), and demonstrated its ability to facilitate efficient MRMI for risk-stratification of EDB-FN-rich prostate and breast cancers, even at doses as low as 20 μmol/kg (⅕th of the clinical dose), highlighting its translational applications and superior safety profile. Despite these and EDB-FN-related studies by independent groups, the expression profiles of EDB-FN in drug-resistant CRC have never been examined. In addition, the feasibility of using EDB-FN as a molecular marker for assessing therapeutic response of drug-resistant CRC remains unexplored.

Here, we demonstrate, for the first time, that MRMI of EDB-FN with MT218 can facilitate efficient non-invasive assessment and treatment response monitoring of drug-resistant CRC tumors that exhibit significantly elevated EDB-FN levels. Subcutaneously implanted drug-resistant CRC xenografts showed robust signal enhancement with 40 μmol/kg dose of MT218, compared to their respective drug-sensitive counterparts. MRMI of EDB-FN was also used to successfully monitor the negative response of drug-resistant CRC tumors to targeted pan-AKT inhibitor, MK2206-HCl (Agarwal, E., et al., BMC Cancer, 2014. 14: p. 145), indicating the potential of EDB-FN as a therapy-predictive marker. Moreover, elevated EDB-FN correlated with poor prognosis of colon cancer patients. To date, this is the first and only report providing compelling in vivo evidence for exploiting drug resistance-mediated upregulation of EDB-FN as a molecular marker for imaging and therapeutic monitoring of CRC.

### Abbreviations

COAD: colon adenocarcinoma; CNR: contrast-to-noise ratio; CRC: colorectal cancer; ECM: extracellular matrix; EDB-FN: extradomain-B fibronectin; EMT: epithelial-mesenchymal transition; GBCA: gadolinium-based contrast agent; 5′-FU: 5′-fluorouracil; GTEx: genotype-tissue expression; MDR1: multidrug resistance 1; MRMI: magnetic resonance molecular imaging; PET-CT: positron emission tomography-computed tomography; TCGA: the cancer genome atlas.

### Methods

### Cell Culture

CRC cell lines DLD-1 and RKO were purchased from ATCC (Manassas, VA). Their respective drug-resistant derivatives, DLD1-DR and RKO-DR were a kind gift from the lab of Zhenghe Wang (CWRU, Cleveland, OH). DLD1-DR cells were developed with resistance to 5′-fluorouracil (5′-FU) (Millipore-Sigma, St. Louis, MO), to an IC₅₀ of 210.6 μM vs IC₅₀ of 2.5 μM for DLD-1 cells, as previously described. Zhu, H., et al., Mol Cancer Ther, 2005. 4(3): p. 451-6. RKO-DR cells were developed with resistance to combined treatment of 10 μM 5′-FU and 15 μM CB-839, a glutaminase inhibitor (Selleck Chemicals, Houston, TX). The DLD-1 and DLD1-DR cells were cultured in McCoy's 5A medium (Thermo Fisher Scientific, Waltham, MA). The RKO and RKO-DR cells were cultured in RPMI1640 medium (Sigma). Both the media were supplemented with 10% fetal bovine serum and 100 Units/mL Penicillin/Streptomycin. All the cells were grown at 37° C. and 5% CO₂.

### Western Blot

Total protein extraction was performed by treating cell pellets with cell lysis buffer (1:1 mix of protease inhibitor in PBS and Laemmli buffer), followed by incubation at 100° C. for 10 min and then centrifugation at 15,000 rpm for 15 min at 4° C. Protein concentration of the extracts was determined using a Lowry assay kit, according to manufacturer's instructions (Bio-Rad, Hercules, CA). Equal amount of protein extracts (40 μg) was loaded on to SDS-PAGE for electrophoresis and transferred onto nitrocellulose membranes. The following primary antibodies (1:1000 dilution, overnight incubation at 4° C.) were used: anti-MDR1, anti-E-cadherin and, anti-R-Actin (Cell Signaling Technology, Danvers, MA) and anti-N-cadherin (1:500 dilution, Abcam, Cambridge, MA). Following secondary antibody incubation (1:2000 dilution for 2 h), the membranes were developed using Signal Fire Plus ECL Kit (Cell Signaling Technology) and imaged on ChemiDoc™ XRS+ Imager (Bio-Rad). The band intensities were quantified using FIJI and ImageLab (Bio-Rad) software. qRT-PCR

Total RNA extraction from the CRC cells was performed using the RNeasy Plus Mini Kit (Qiagen, Germantown, MD), according to manufacturer's protocol. cDNA was generated by reverse transcription with the miScript II RT Kit (Qiagen) and qPCR was performed using SyBr Green PCR Master Mix (Thermo Fisher Scientific). Relative gene expression was measured by the 2-ΔΔCt method. R-Actin was used as the housekeeping gene.

### Invasion and Migration Assays

Standard transwell assays were performed to assess the migration and invasion of CRC cells. To test migration, 100,000 CRC cells (starved overnight) were plated in transwell inserts (VWR, Radnor, PA). The next day, the inserts were internally swabbed to remove the non-migrated cells. The migrated cells at the bottom of the inserts were fixed with 10% formalin (10 min) and then stained with 0.1% crystal violet (20 min). Excess stain was washed and the transwells were dried overnight before imaging on the Moticam T2 camera with 10× objective lens. To test invasion, the transwell inserts were coated with 1 mg/mL Corning™ Matrigel™ Membrane Matrix (Corning, NY), to assess the ability of the CRC cells to invade through the Matrigel layer, in addition to the porous membrane of the inserts. Approximately 200,000 CRC cells (starved overnight) were plated for this assay and processed as mentioned above. The invading and migration cells were quantified using FIJI (FIJI is Just ImageJ) software.

### 3D Culture and ZD2-Cy5.5 Staining

The ability of CRC cells to grow in 3D culture was tested using Matrigel culture. About 900,000 CRC cells were plated in 4-well microslides (Ibidi, Fitchburg, WI) coated with a thick layer of Corning™ Matrigel™ Membrane Matrix. Tumor spheroid/organoid formation was monitored and photographed for up to 4 days using the Moticam T2 camera with 10× objective lens. To test EDB-FN expression, the tumor spheroids were incubated with 100 nM ZD2-Cy5.5 and 5 μg/mL Hoechst-33342 for 30 min. Excess dyes were washed thrice with PBS and fluorescence imaging was performed on Olympus FV1000 confocal microscope (Japan), with 10× and 20× objective lenses. Image processing was done using FIJI.

### Mouse Models

Nude athymic mice (6-week-old nu/nu females) were purchased from The Jackson Laboratory (Bar Harbor, MA) and housed in the Animal Facility at CWRU. All the animal experiments were performed according to the protocol approved by the IACUC of CWRU. For assessment of drug resistance, 2 drug-resistant and 2 non-drug-resistant models were set up. About 3-4×10⁶ DLD-1, RKO, DLD1-DR, and RKO-DR cells suspended in Matrigel-PBS mixture (1:1) were subcutaneously injected in the left flanks of nude mice (100 μL per mouse, 5 mice per group×4 models=20 mice). After 9 days when the tumors reached 100-200 mm3, MRMI was performed on the 4 xenograft models with 40 μmol/kg dose of MT218. The animals were then euthanized, and the tumors were harvested for post-mortem histology and IHC.

For therapeutic monitoring, 3-4×10⁶ DLD1-DR cells suspended in Matrigel-PBS mixture (1:1) were subcutaneously injected in the left flanks of 10 nude mice (100 μL per mouse, mice labeled TM1-TM10). Tumor volumes were monitored and measured once a week using a Vernier caliper. When the average tumor volumes reached 100 mm³, mice were randomized into 2 groups of 5: vehicle (mice #TM1, TM3, TM4, TM5, & TM10) and treated (mice #TM2, TM6, TM7, TM8, & TM9). Once a week, mice in the treated group received MK2206-HCl (100 mg/kg) and those in the vehicle group were injected with equivalent volume of DMSO, as described previously. Smith, J. A., L. J. Stallons, and R. G. Schnellmann, Am J Physiol Renal Physiol, 2014. 307(4): p. F435-44. After 3 weeks of treatment, tumor volumes increased over 1000 mm³ and the experiment was terminated. The animals were then euthanized, and the tumors were harvested for post-mortem histology and IHC. Tumor volumes were calculated as [(Width)2×Length]/2.

MRMI of EDB-FN with MT218

MT218 was synthesized as previously described. Ayat, N. R., et al., ACS Med Chem Lett, 2018. 9(7): p. 730-735. Briefly, click reaction between alkynyl-ZD2 and N3-Gd(HP-DO3A) was performed in the presence of CuSO₄ and ascorbate at room temperature, followed by FLASH chromatography purification and validation of MT218 by MALDI-TOF mass spectrometry (m.w. 1443). For assessment of drug resistance and therapeutic monitoring, MRMI was performed in a 3T MRS 3000 scanner (MR Solutions, Surrey, UK) with a mouse short quad coil. The mice were anesthetized with isofluorane and tail vein catheter was setup. T1-weighted MR images were obtained before (pre-contrast) and 25 min after injection (post-contrast) of 40 μmol/kg dose of MT218 [ZD2-N₃—Gd(HP-DO3A)]. The following two sequences were used with respiratory gating: axial fast spin echo (FSE) (TR=305 ms, TE=11 ms, FA=90°, FOV=40 mm×40 mm, slice thickness=1 mm, slice number=15, Nav=2, matrix=256×256) and coronal FSE (TR=305 ms, TE=11 ms, FA=90°, FOV=90 mm×90 mm, slice thickness=1 mm, slice number=20, Nav=1, matrix=248×512). For therapeutic monitoring, baseline MRMI (week 1) and endpoint MRMI (week 4) were performed using the aforementioned sequences with 40 μmol/kg dose of MT218 on the 10 mice bearing DLD1-DR tumors. Contrast-to-noise ratios (CNRs) were calculated as (mean tumor intensity—mean muscle intensity)/standard deviation of noise. Image and CNR analysis was performed using FIJI software. ROIs were drawn around whole tumor, 2-4 muscle regions, and background. CNR analysis was performed independently by 2 individuals, once blinded, to avoid bias.

### Immunohistochemistry in Human and Mouse Tissues

De-identified and de-classified human tissue samples, including primary colon adenocarcinoma (n=6), liver metastasis (n=4), and their corresponding normal adjacent tissues, (n=6+4), were acquired from the Human Tissue Procurement Facility at CWRU. Dissected mouse tumor tissues were fixed in 10% neutral buffered formalin, embedded in paraffin, and sectioned into 1 μm slices. Staining and IHC services were provided by the Tissue Resources Core Facility of the Case Comprehensive Cancer Center and University Hospitals of Cleveland. The slides were stained with H&E to visualize morphology. Immunohistochemistry was performed using anti-EDB-FN antibody G4 clone (1:100 dilution; Absolute Antibody, UK), as previously described. All the slides were reviewed by a certified pathologist. IHC images were obtained using Bx61VS slide scanner microscope (Olympus) with 40× objective lens and processed in OlyVIA software.

### Gene Data and Statistical Analyses

Kaplan-Meier curves for overall survival (OS) and proliferation-free survival (PFS) data for correlation with EDB-FN expression (transcript ID: ENST00000432072.6) were derived in GEPIA2. Tang, Z., et al., Nucleic Acids Res, 2017. 45(W1): p. W98-W102. This web server evaluates tumor/normal data and normal tissue data (transcript per million) from TCGA and GTEx databases, respectively, and employs Log-rank, or Mantel-Cox test, for statistical survival analysis and Cox PH Model for hazards ratio (HR) calculation.

All the experiments were independently replicated at least 3 times (n=3), unless otherwise stated. Data are represented as mean±s.e.m. Statistical analysis was performed using GraphPad Prism version 7.03. Data between two groups (normal distribution) was compared using unpaired t-test. Otherwise, non-parametric test (Mann-Whitney U test) was used, as stated in the relevant figure legends. Time course MRMI data for multiple cell lines was analyzed by 2-way analysis of variance (ANOVA) with Tukey's correction. p<0.05 was considered to be statistically significant.

### Results

### Acquired Drug Resistance Enhances Migration and Invasion of CRC Cells

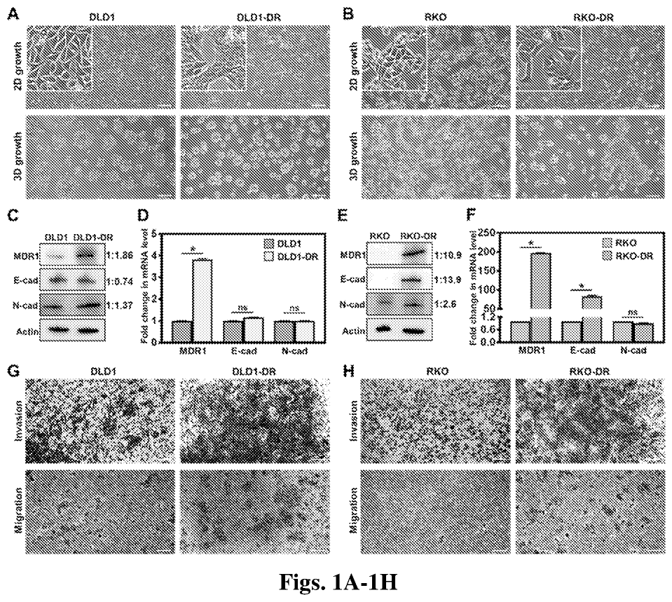

Two independent drug-resistant CRC lines were generated: DLD1-DR by long-term 5′-FU treatment in DLD-1 cells and RKO-DR by combined treatment of 5′-FU and CB-839 in RKO-DR cells, and evaluated for their biological properties. The morphology of cells grown in 2D and 3D cultures was monitored by phase contrast microscopy. While DLD-1 cells showed regular epithelial morphology in 2D culture and multicellular grape-like clusters in 3D culture (FIG. 1A), DLD1-DR cells formed islets in 2D culture and compact spheroids in 3D culture. Monolayer cultures of RKO and RKO-DR cells showed no overt morphological differences (FIG. 1B). On the other hand, in 3D culture, RKO-DR cells readily formed tumor spheroids, both compact and aggregates, while RKO cells were unable to form spheroids. These results demonstrate the diverse morphological variation, both in monolayer and 3D culture, between the CRC cell lines.

Next, drug resistance-induced molecular changes in the signaling pathways of the CRC cells were analyzed by western blotting and qRT-PCR. Since drug efflux pumps and epithelial-mesenchymal transition (EMT) are implicated in the development of drug resistance, the protein and mRNA expression of multidrug resistance protein 1 (MDR1), and EMT markers (E-cadherin and N-cadherin) were determined. At the protein level, DLD1-DR cells showed significant upregulation of MDR-1 and moderate EMT, with modest decrease in E-cad and increase in N-cad levels, over DLD-1 cells (FIG. 1C). At the mRNA level, the DLD1-DR cells showed 4-fold increase in MDR-1, and with no changes in mRNA levels of E-cad and N-cad (FIG. 1D). On the other hand, RKO-DR showed significantly elevated MDR1 and E-cad at both protein (FIG. 1E) and mRNA (FIG. 1F) levels, over RKO cells. The N-cad protein expression was also elevated in RKO-DR cells, suggesting the existence of a mix of epithelial and mesenchymal cells or a partial/hybrid E-M phenotype in RKO-DR cells.

The functional effects of acquired drug resistance in the CRC cells were evaluated by testing their migratory potential. As shown in FIG. 1G-H and Figure S1B-C, DLD1-DR and RKO-DR cells exhibited increased migration through transwell membranes as well as increased invasion through an additional layer of Matrigel coated on the transwell membranes. Taken together, these results suggest that irrespective of the distinct morphological and molecular changes caused by acquisition of drug resistance, CRC cells gain significant invasive advantages over their parent counterparts.

Acquired Drug Resistance is Associated with Elevated EDB-FN Expression in CRC Cells

We previously showed that EDB-FN is overexpressed in drug-resistant, invasive breast cancer (Han, Z., et al., Nat Commun, 2017. 8(1): p. 692) and aggressive prostate cancer. Han, Z., et al., Bioconjug Chem, 2017. 28(4): p. 1031-1040. Here, we evaluated whether development of drug resistance upregulates EDB-FN expression in the CRC cells, using qRT-PCR and EDB-FN-specific peptide probe ZD2-Cy5.5 (Han, Z., et al., Bioconjug Chem, 2015. 26(5): p. 830-8) in 2D and 3D cultures, respectively.

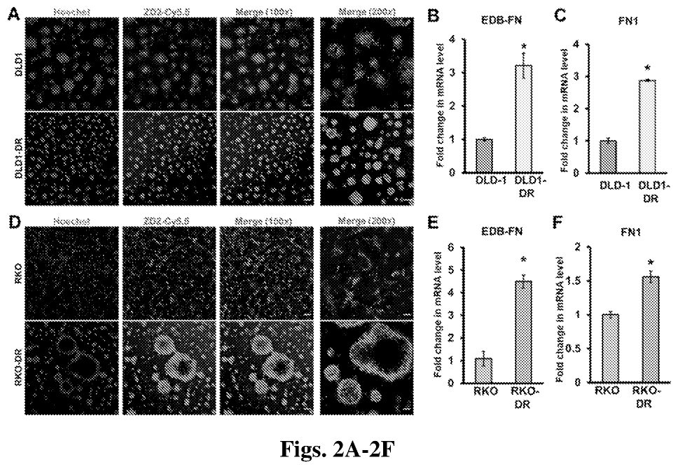

As shown in FIG. 2A, both DLD-1 and DLD1-DR cells expressed EDB-FN, evident in the ZD2-Cy5.5 binding. However, the DLD1-DR cells showed brighter fluorescence signal, indicating higher ZD2-Cy5.5 binding, which was confirmed by the 3-fold upregulation of EDB-FN mRNA level over DLD-1 cells (FIG. 2B). Total FN1 expression was also found to increase with drug resistance in DLD1-DR cells (ca. 3-fold, FIG. 2C). Similarly, while both RKO and RKO-DR cells secreted EDB-FN, its expression was significantly higher in the latter, reflected in the intense ZD2-Cy5.5 staining (FIG. 2D) and approximately 4.5-fold increase in EDB-FN mRNA (FIG. 2E), over the former. Total FN1 was only increased by 1.5-fold in the RKO-DR cells (FIG. 2F). These results demonstrate that acquired drug resistance in CRC cells is associated with EDB-FN overexpression.

### Contrast-Enhanced MRMI of EDB-FN Using MT218 Facilitates Effective Differential Diagnosis of Drug Resistant CRC Tumors

To determine whether EDB-FN overexpression can be used as a molecular marker to differentiate between non-resistant and drug-resistant CRC tumors, MRMI was performed using EDB-FN-targeting contrast agent MT218 in athymic nu/nu mice bearing subcutaneous xenografts of DLD-1, DLD1-DR, RKO, and RKO-DR. T1-weighted coronal and axial images were acquired before and 25 min after injection of 40 μmol/kg MT218. We previously showed that this subclinical dose of MT218 is just as effective as the standard dose (0.1 mmol/kg) of MT218 and the clinical contrast agent Gadoteridol. Ayat, N. R., et al., Front Oncol, 2019. 9: p. 1351.

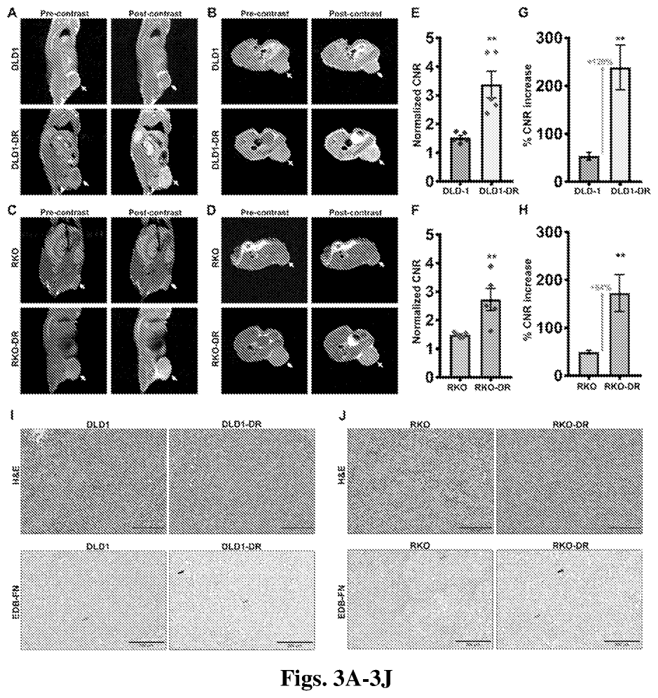

Preliminary time course analysis showed robust enhancement and increased CNRs up to 35 min post-injection of MT218 in both the DLD-1 and RKO tumor models. Since the peak enhancement was observed between 20-35 min, the 25 min time point was selected for the subsequent assessment of drug resistance. MRMI of EDB-FN using MT218 resulted in signal enhancement in the non-resistant DLD-1 and RKO tumors (FIG. 3A-D), with 1.53- and 1.4-fold increase in CNRs over the pre-contrast, (FIG. 3E-F), respectively. The drug-resistant DLD1-DR and RKO-DR tumors exhibited significantly stronger signal enhancement, reflected in the 3.3- and 2.6-fold higher CNRs over pre-contrast (FIG. 3A-F). Importantly, this signal enhancement was more robust than in their non-resistant counterparts, resulting in 2.2- and 1.8-fold higher CNRs (i.e., 120% and 84% increase) over the non-resistant DLD-1 and RKO tumors, respectively (FIG. 3E-H). The mice were euthanized post-imaging and their tumor tissues were processed for H&E staining and EDB-FN immunohistochemistry (IHC) with G4 antibody. As shown in FIG. 3I-J, H&E staining did not show significant differences between the drug-resistant and non-resistant CRC tumors, with both demonstrating high grade, poorly differentiated, and mitotically active pleomorphic cancer cells. IHC for EDB-FN exhibited stronger staining in DLD1-DR (FIG. 3I) and RKO-DR (FIG. 3J) tumors, compared to that in the DLD-1 and RKO tumors, respectively. EDB-FN staining was localized in both, the tumor cells and the elongated spindle-like interspersed cancer-associated fibroblasts (CAFs), denoted by the red and black arrows, respectively. This IHC data correlated with the signal enhancement and CNRs observed in the four models, demonstrating MRMI of EDB-FN with reduced dose of MT218 can facilitate non-invasive assessment of drug resistance in CRC models.

### MRMI of EDB-FN Using MT218 Facilitates Therapeutic Response Monitoring of Drug-Resistant CRC Tumors

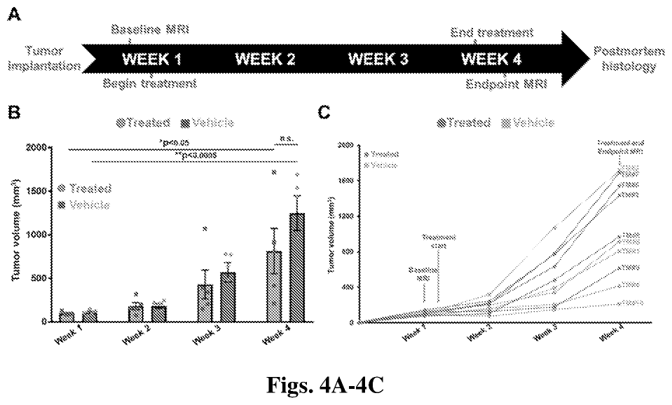

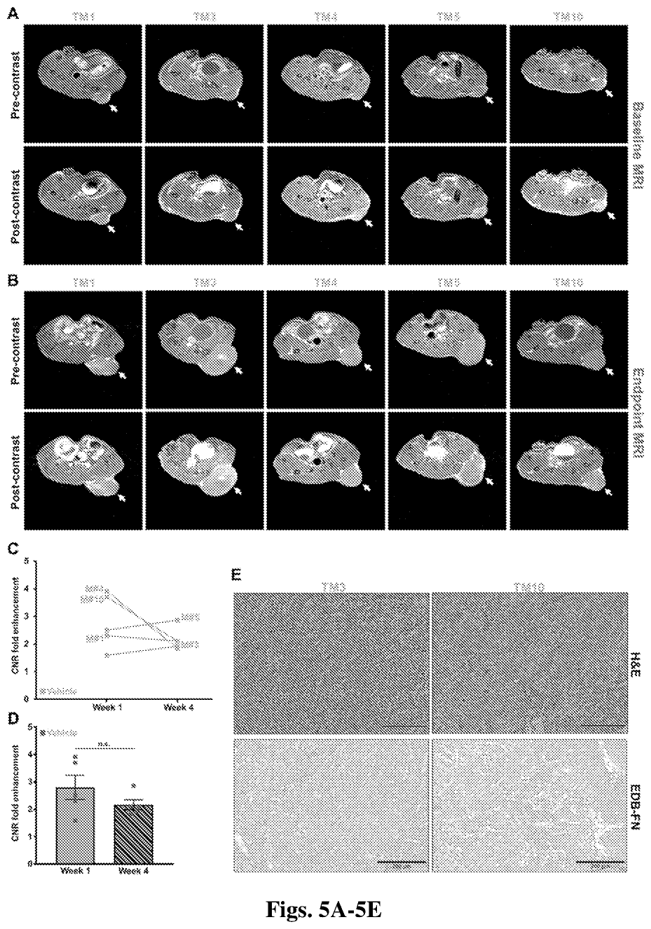

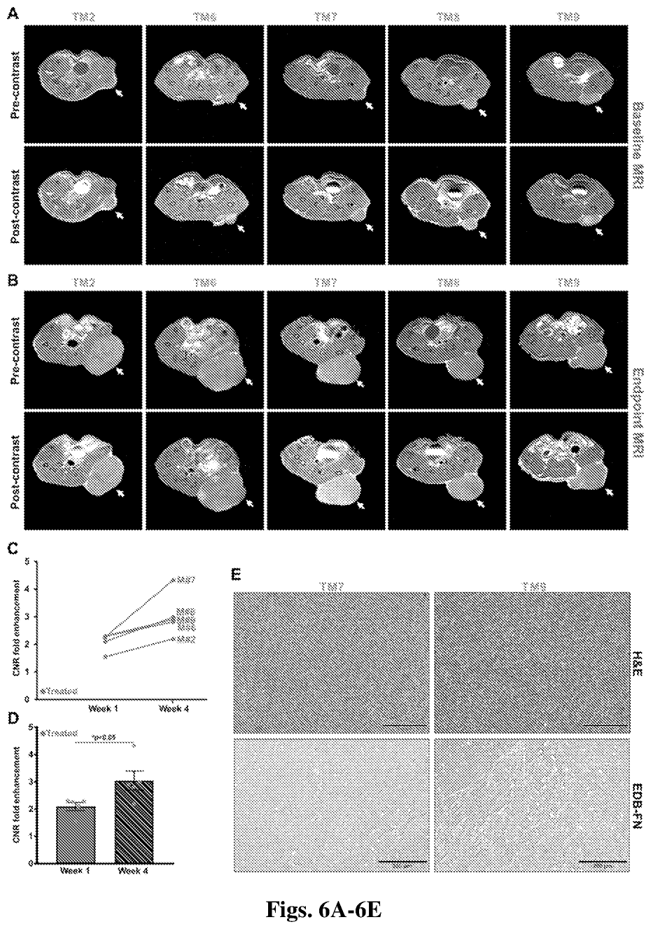

Following differential diagnosis of drug-resistant CRC tumors, the potential of MRMI of EDB-FN for non-invasive assessment of therapeutic response was determined in DLD1-DR-bearing mice treated with MK2206-HCl, a pan-AKT inhibitor. FIG. 4A shows the schematic of the timeline of therapeutic regimen and MRMI for therapeutic efficacy monitoring. One week after tumor implantation, baseline MRMI was performed, followed by randomization of the mice into vehicle (DMSO-treated: TM1, TM3, TM4, TM5, TM10) and treated (MK2206-treated: TM2, TM6, TM7, TM8, TM9) groups. Mice were treated once a week for 3 weeks, and endpoint MRMI was performed at week 4 due to high tumor burden, followed by post-mortem histology. Tumor volumes were monitored once a week. FIG. 4B shows the progression of tumor growth over the 4-week period. The average tumor volumes of both vehicle and treated groups increased significantly, from 97.7±8.14 mm³ to 813.4±259.7 mm³ and from 110.5±11.97 mm³ to 1249.7±199.3 mm³, respectively. Although no significant difference was found between the final average tumor volumes of the vehicle and treated groups (p=0.22), individual mice in the latter group showed visibly worse and larger tumors, indicating that the drug-resistant tumors did not respond to therapy. The tumor volumes of individual mice were also monitored (FIG. 4C) to correlate with the endpoint MRMI signal and EDB-FN expression. Each mouse (labeled from TM1-TM10) showed significant increase in tumor volumes, to a lesser or greater extent.

The tumor growth was monitored with endpoint MRMI for EDB-FN using MT218. As shown in FIG. 5A, each mouse in the vehicle group showed signal enhancement compared to pre-contrast, when baseline MRMI was performed. After 3 weeks of DMSO injections, all the 5 mice showed signal enhancement in endpoint MRMI; with mice #TM3 and TM5 showing increased signal enhancement and tumor size, compared to the other 3 mice (TM1, TM4, and TM10), FIG. 5B. Quantification of this data showed a consistent trend, with TM3 and TM5 mice showing increase in CNRs from week 1 to 4, and TM1, TM4, and TM10 mice showing decrease in CNRs from week 1 to 4 (FIG. 5C). The MRMI signal also correlated with the increase in their tumor volumes, where TM3 and TM5 mice showed a rapid increase in tumor volumes compared to TM1, TM4, and TM10 mice (FIG. 4C). No significant difference was found between the average CNRs of the vehicle group from week 1 to 4 (FIG. 5D). Postmortem histology and IHC for EDB-FN showed stronger G4 staining in TM3 than TM10, demonstrating MRMI of EDB-FN with MT218 correlates with the endogenous tumor EDB-FN expression (FIG. 5E).

As for the treated group, each of the 5 mice showed high signal enhancement over pre-contrast during baseline MRMI (FIG. 6A). Despite 3 weeks of treatment, the drug-resistant DLD1-DR tumors failed to respond to therapy and increased in size with increased signal enhancement over pre-contrast (FIG. 6B), reflected in the elevated CNRs for the individual mice from week 1 to week 4 (FIG. 6C). Among the 5 mice, mouse #TM7 showed the highest signal enhancement and CNR, and the largest tumor volume (FIG. 4C). On the other hand, mice #TM2, TM6, TM8, and TM9 showed increased CNRs and tumor volumes from week 1 to 4, but no correlating pattern between the two was detected. The average tumor CNR in the treated group increased from week 1 to 4 (FIG. 6D), confirming that the drug-resistant tumors failed to respond to therapy, and performed worse than the vehicle group. Postmortem histology and IHC for EDB-FN showed strong G4 staining for the tumor in mouse #TM7, compared to TM9 (FIG. 6E) as well as vehicle-treated mice #TM3 and TM10 (FIG. 5E), further corroborating the direct correlation of EDB-FN expression and MRMI-based monitoring. While the week 4 average CNR of the treated group was higher than that of the vehicle group, it was not statistically significant (p=0.055, Figure S4). Taken together, these results demonstrate that MRMI of EDB-FN with MT218 can facilitate non-invasive therapeutic monitoring of drug-resistant CRC tumors.

EDB-FN is Overexpressed in Human Colon Adenocarcinoma and is Correlated with Poor Patient Survival

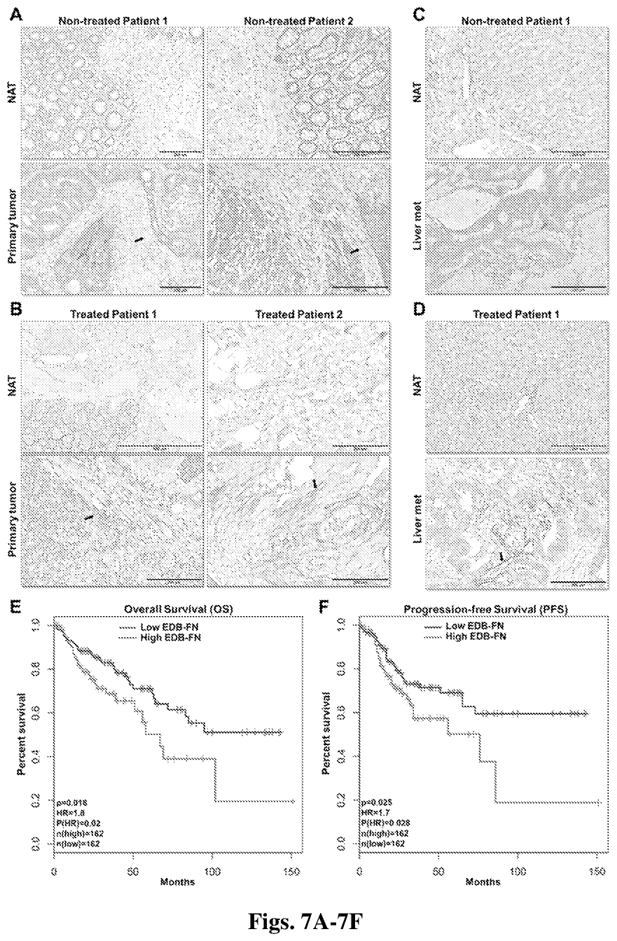

To assess the feasibility of using MRMI of EDB-FN in CRC patients, the expression of EDB-FN was analyzed in representative human specimens of colon adenocarcinoma (COAD), metastatic liver, and their corresponding normal adjacent tissues. Primary COAD tumors were found to demonstrate strong EDB-FN expression in both untreated (FIG. 7A) and treated (FIG. 7B) patients, with negligible expression in the corresponding normal adjacent tissues. On the other hand, COAD hepatic metastases showed differential EDB-FN levels, with moderate staining in non-treated patient (FIG. 7C) and intense staining in treated patient (FIG. 7D), and no staining in normal adjacent liver. In regards to the pattern, most of the EDB-FN staining was observed in the stromal and cancer-associated fibroblasts (black arrows), and some in the tumor cells (grey arrows), in the primary and metastatic sites. Moreover, analysis of RNA-Seq data from TCGA and GTEx databases revealed that increased EDB-FN levels correlated with poor overall survival (OS) (FIG. 7E) and poor progression-free survival (FIG. 7F) of COAD patients, suggesting that EDB-FN overexpression could be a prognostic indicator for COAD.

### Discussion

This work demonstrates that invasive drug-resistant CRC tumors overexpress the ECM protein EDB-FN, compared to their non-resistant counterparts. Drug-resistant CRC tumors that show negative response to targeted therapy also upregulate EDB-FN. These therapeutic pressure-induced alterations in EDB-FN levels enable effective non-invasive assessment and treatment response monitoring of drug-resistant CRC by MRMI, even at subclinical doses of MT218.

The molecularly diverse landscape of CRC underscores the need for robust oncomarkers that can provide diagnostic, prognostic, or therapy-predictive value. Here, we showed, for the first time, that overexpression of EDB-FN correlated negatively with CRC patient survival. A prior study that showed poor prognosis of post-operative CRC patients with high oncFN1 levels used FDC-6 antibody, which binds to an alternative oncofetal isoform of FN1. Inufusa, H., et al., Cancer, 1995. 75(12): p. 2802-8. Immunohistochemical analysis of patient samples showed strong EDB-FN specific staining with G4 antibody in the primary and metastatic CRC sites. EDB-FN was found to be localized in the stroma, stromal fibroblasts, adenocarcinoma cells, and fibroblasts interspersed around these tumor cells. These results validate the recently emerging consensus that multiple cell types in the tumor milieu express EDB-FN (Midulla, M., et al., Cancer Res, 2000. 60(1): p. 164-9), suggesting multifaceted interactions of the cellular and extracellular components of the tumor microenvironment. The profound tumor-specific expression of EDB-FN was evident from the complete absence of G4 staining in the normal adjacent colon and liver tissues, signifying EDB-FN as an attractive candidate marker for CRC. The detection of over 300% increase in EDB-FN levels in the urinary samples of muscle-invasive bladder cancer patients, and a negative correlation to their clinical outcomes, also validates its potential as a promising diagnostic oncomarker for other cancers. Arnold, S. A., et al., Clin Exp Metastasis, 2016. 33(1): p. 29-44

Both the 2D and 3D cultures demonstrated a positive association between the invasiveness of drug-resistant CRC cells and their endogenous EDB-FN expression. Between the two non-drug-resistant models, the DLD-1 cells exhibited higher expression of EDB-FN than the RKO cells. This is consistent with the inherent biological properties of the models, wherein the DLD-1 cells derived from Duke's type C, p53-mutated, CEA-expressing adenocarcinoma are more invasive and migratory than the RKO cells derived from a p53-, K-Ras-wild type primary carcinoma site. Gu, C., et al., Cell Death Dis, 2018. 9(6): p. 654. Development of resistance to 5′-FU by DLD-1 and 5′-FU+CB-839 by RKO cells resulted in dynamically altered EMT-like DLD1-DR and hybrid E-M RKO-DR cells, highlighting the distinct trajectories taken by the 2 cell lines following therapeutic pressure-induced clonal selection. Both the drug-refractory cell lines also exhibited robust overexpression of the ATP-binding cassette (ABC) transporter MDR-1, which is unsurprising given that enhanced cellular efflux is a common mechanism of 5′-FU-resistance. Skarkova, V., et al., Cells, 2019. 8(3). It is likely that the increased plasticity induced by the hybrid E-M phenotype confers CB-839 resistance on the RKO-DR cells. Enhanced plasticity and metabolic redundancy have been previously observed in CB-839-resistant breast cancer, although the precise mechanisms of glutaminase inhibition resistance in CRC remain understudied. Reis, L. M. D., et al., J Biol Chem, 2019. 294(24): p. 9342-9357.

Irrespective of the heterogeneous molecular changes and EMT status, acquisition of invasive and migratory properties by both the drug-resistant cell lines was associated with significant upregulation of EDB-FN. This upregulation was recapitulated in the drug-resistant DLD1-DR and RKO-DR xenografts, which exhibited robust signal enhancement and significantly higher CNRs in MRMI with EDB-FN-targeting contrast agent MT218, over their respective non-resistant counterparts, suggesting the potential of EDB-FN as a promising diagnostic oncomarker. We previously showed overexpression of EDB-FN in drug-resistant breast cancer cells. Other groups also demonstrated elevated EDB-FN in CRC cells and tumors. El-Emir, E., et al., Br J Cancer, 2007. 96(12): p. 1862-70. To our knowledge, this is the first report demonstrating EDB-FN overexpression in drug-refractory CRC. These elevated EDB-FN levels could be leveraged for ZD2-targeted MRMI-mediated non-invasive surveillance of tumor progression, to identify critical events and stages that precede the development of drug resistance.

Drug resistance is the bane of oncotherapy and cancer radiology. Accurate non-invasive assessment of these drug-resistant cells and tumor response to oncotherapy (early within few days of treatment) has the potential to revolutionize the contemporary cancer treatment paradigm. Multiple lines of clinical evidence now indicate that assessment of therapeutic response cannot be based on the change of tumor size alone. Consequently, we hypothesized that molecular imaging of abundantly overexpressed EDB-FN in correlation with the aggressive nature of the disease and independent of tumor size can potentially provide accurate tumor response to therapies. In our treatment regimen, the failure of the highly drug-resistant DLD1-DR tumors to MK2206-HCl therapy was reflected in their increased EDB-FN levels, and a consequent increase in MT218 uptake, demonstrated by robust signal enhancement and increased CNRs in MRMI. These tumors also increased in size, as did the vehicle tumors; however, the latter did not show comparable increase in EDB-FN and MT218 uptake as the treated tumors, suggesting that the MRMI was based on inherent EDB-FN expression and not anatomical tumor size. While the negative response to MK2206-HCl was unexpected considering our findings on the role of phospho-AKT signaling in EDB-FN regulation in breast cancer cells, we speculate that the enhanced plasticity and overexpression of the drug efflux pump MDR-1 could have rendered the DLD1-DR tumors non-responsive to MK2206-HCl. A compensatory activation of alternative AKT-independent oncogenic signaling (e.g., NF-κβ) in 5′-FU-treated DLD-1 cells has also been shown before [62]. The treatment was terminated at week 4 due to the large tumor burden, precluding any other adaptive interventions or imaging tests. Although this research was limited to the imaging of primary tumors, the promising results warrant further investigation in to analyzing additional parameters and imaging modalities in conjunction with monitoring tumor volumes to validate and correlate the tumor responses to various therapies.

Only two previous reports on EDB-FN-based radioimmunotherapy in CRC using ¹²⁵I labeled L19-SIP antibody exist, which demonstrated selective tumor uptake and tumor growth inhibition in CRC xenografts (El-Emir, E., et al., Br J Cancer, 2007. 96(12): p. 1862-70) and CRC patients (Santimaria, M., et al., Clin Cancer Res, 2003. 9(2): p. 571-9); however, EDB-FN-based MRMI assessment of drug-resistant CRC has never been performed before. Our preliminary data serve as a groundwork for transforming EDB-FN-targeted molecular imaging, alone and as an adjunct to other screening modalities, into a clinically viable technology for CRC management. One of the challenges of MRI in CRC imaging is accurate detection of hepatic CRC metastases, a major prognostic indicator of patient outcomes. Given that we and independent researchers observed elevated EDB-FN expression in patient metastatic liver specimens, accurate and timely detection of these evasive niches could provide a crucial diagnostic advantage during treatment regimens. Although MRI is the primary modality of choice for staging rectal cancer, it's performance for tumor restaging following oncotherapy is inconsistent. Therefore, it would be interesting to evaluate the efficacy of MRMI with MT218 in determining the response of patients to neoadjuvant therapy during the “wait and watch” period, before planning surgical interventions.

Although contrast-enhanced MRI remains crucial for diagnosis, cumulative exposure to contrast media, especially linear GBCAs, results in deposition and long-term accumulation of Gd in the brain, bones, and even skin of patients. MT218 is a small peptide conjugate of a clinical macrocyclic contrast agent Gadoteridol with high stability and good safety profile. We recently demonstrated strong tumor-specific enhancement in MRMI with MT218 at a reduced dosed of 40 μmol/kg in comparison to the standard clinical dose of 0.1 mmol/kg in breast cancer models. Ayat, N. R., et al., Front Oncol, 2019. 9: p. 1351. The uncompromised efficacy of MRMI with MT218 at the reduced dose can minimize the potential risk of Gd-associated and dose-dependent toxicity. This low dose was also sufficient to enable effective assessment of CRC and drug-resistant CRC tumors in this study. By virtue of the high T1 relaxivity (6.13 mM⁻¹s⁻¹ at 3T) of MT218, effective MRMI at subclinical doses, and easy accessibility of abundant EDB-FN in the tumor microenvironment to MT218 binding, we posit that MRMI with reduced Gd exposure is promising for active surveillance and treatment monitoring of CRC. Given that tumor cell plasticity engenders extensive patient-to-patient variability in tumor progression and therapeutic response, even within the same cancer type, MRMI with MT218 based on the tumor levels of EDB-FN could detect the development of drug resistance early during the chemotherapeutic period, helping to tailor the treatment regimen for the relevant patients and improving the success of oncotherapy.

The unique advantages of EDB-FN-specific ZD2 peptide can also be harnessed to develop integrated imaging systems like PET/MRI or PET/CT for multi-parametric molecular imaging. Indeed, PET and SPECT probes have already been generated by conjugating the EDB-FN-specific ZD2 peptide to radiotracers like ⁶⁴Cu-DOTA, ⁶⁸Ga-NOTA, and ^{99m}Tc-HYNIC chelates for improved detection of prostate, pancreatic, and breast cancers, respectively. Han, Z., et al., ACS Omega, 2019. 4(1): p. 1185-1190; Gao, S., et al., Am J Nucl Med Mol Imaging, 2019. 9(5): p. 216-229. These systems could also provide insight into several aspects of CRC, including metastatic surveillance following primary tumor resection, imaging of CRC in syngeneic models treated with chemotherapy, and assessment of tumor response to immunotherapy. Besides ZD2, other EDB-FN-targeting ligands, antibodies and nanobodies, including APT-FN-EDB, L19, BC1, and NJB2 have also been used to deliver therapeutic and imaging agents to tumor ECM and vasculature, validating the role of EDB-FN as a promising oncomarker. Tijink, B. M., et al., Eur J Nucl Med Mol Imaging, 2009. 36(8): p. 1235-44; Jailkhani, N., et al., Proc Natl Acad Sci USA, 2019. 116(28): p. 14181-14190.

In summary, we demonstrate tumor-specific EDB-FN expression in colon adenocarcinoma (COAD) specimens, and poor prognosis of COAD patients with high levels of EDB-FN. While CRC cells and tumor xenografts inherently express EDB-FN, its expression is further elevated in their drug-resistant counterparts. MRMI of EDB-FN with a significantly reduced dose of MT218 can facilitate effective non-invasive assessment and therapeutic monitoring of drug-resistant CRC, highlighting the translational potential of MT218-mediated EDB-FN-targeting MRMI in active surveillance and monitoring of drug-resistant neoplasms.

### Example 2: Overexpression of Extradomain-B Fibronectin is Associated with Invasiveness of Breast Cancer Cells

Breast cancer (BCa) is a devastating disease that accounts for 41,000 deaths each year in the US. Although the survival rate for patients with localized BCa is close to 99%, it declines precipitously in patients with distant metastases and drug resistance. A major stumbling block in the clinical management of the disease is tumor heterogeneity, which plays a role in the dynamic nature of BCa progression. Whole genome sequencing and profiling studies have demonstrated that breast tumors of the same histological subtype exhibit distinct molecular portraits and discrete trajectories in individual BCa patients at different stages. Stochastic mutations, genome instability, and clonal evolution arising from selective pressures from genetic, epigenetic, environmental, and therapeutic stimuli result in the emergence of high-risk tumor populations with significant growth and invasive advantages. This extensive spatial and temporal diversity within primary and metastasized tumors directly influences diagnostic, therapeutic, and prognostic outcomes. In the absence of markers specific to the metastatic and invasive properties of tumors, current imaging modalities including MRI, PET, and CT are limited in their ability to detect and differentiate between low-risk and high-risk tumors. These facts underscore the need for the discovery and characterization of suitable molecular markers that can facilitate non-invasive detection, risk-stratification, active surveillance of breast neoplasms, and timely assessment of therapeutic response, despite their dynamic nature.

The tumor extracellular matrix (ECM) plays a critical role in all aspects of tumor progression, by relaying oncogenic signals between the tumor cells and the tumor microenvironment (TME) and by supporting growth, apoptotic escape, migration, inflammation, and immune evasion. Fibronectin (FN1), an integral component of normal and tumor ECM, is an essential glycoprotein that regulates adhesion, motility, growth and development. Its alternative splice variant called extradomain-B fibronectin (EDB-FN), however, is known to be expressed during malignant transformation, and is generally absent from healthy adult tissues. Han, Z., and Lu, Z. R., J Mater Chem B 5, 639-654 (2017). Multiple lines of evidence show that EDB-FN is associated with epithelial-to-mesenchymal transition (EMT), cancer cell stemness, proliferation, angiogenesis, and metastasis, all of which reflect tumor aggressiveness. Han, et al., Magn Reson Med 79, 3135-3143 (2018). Clinical studies demonstrate the presence of EDB-FN in patients with lung, brain, colorectal, and ovarian cancers. Santimaria et al., Clin Cancer Res 9, 571-579 (2003). The overexpression of EDB-FN is also correlated with histological grade in mammary tumors (Loridon-Rosa et al., Cancer Res 50, 1608-1612 (1990)) and with poor survival in oral carcinoma patients (Lyons et al., Br J Oral Maxillofac Surg 39, 471-477 (2001)), suggesting its potential role as a marker for multiple neoplasms.

An added layer of complication is that even among the same cancer type, EDB-FN expression profiles are distinct and specific to the molecular and functional characteristics of the cells or tissues. For example, using an EDB-FN-specific peptide probe, ZD2-Cy5.5, we previously showed that invasive cancer cell lines, e.g., PC3 (prostate) and MDA-MB-231 (hormone receptor-negative breast cancer), are EDB-FN-rich, while the less invasive cancer cell lines, e.g., LNCaP (prostate) and MCF7 (hormone receptor-positive breast) exhibit significantly lower EDB-FN levels. Han et al., Bioconjug Chem 26, 830-838 (2015); Han et al., Nat Commun 8, 692 (2017). This differential expression of EDB-FN was exploited for differentially diagnosing invasive prostate and breast cancer tumors from the non-invasive xenografts using EDB-FN-targeted MRI contrast agents. Ayat et al., ACS Med Chem Lett 9, 730-735 (2018). Other independent groups have also used EDB-FN as a molecular target for targeted imaging and therapeutic delivery for various types of cancer. Sun et al., Theranostics 4, 845-857 (2014); Ye et al., ACS Omega 2, 2459-2468 (2017).

Given the high degree of tumor plasticity, it is evident that different selective pressures, environmental and experimental stimuli will bring about distinct changes in the TME and EDB-FN expression, which would in turn influence the clinical outcomes of EDB-FN-targeted imaging and therapeutic interventions. Here, we sought to determine the changes in EDB-FN expression patterns following application of two different selective pressures on non-invasive, low EDB-FN-expressing breast cancer cells and their consequent evolution into invasive high-risk populations. To this end, significant survival advantage was conferred on two breast cancer cell lines, MCF7 and MDA-MB-468, by treating them with the cytokine TGF-β and chemotherapeutic drugs to induce stochastic alternations and clonal evolution. The resulting populations were also treated with a highly specific AKT inhibitor to further assess for changes in EDB-FN levels in correlation to the response of the high-risk cells to targeted therapy.

### Results

### EDB-FN Expression is Significantly Elevated in Breast Cancer

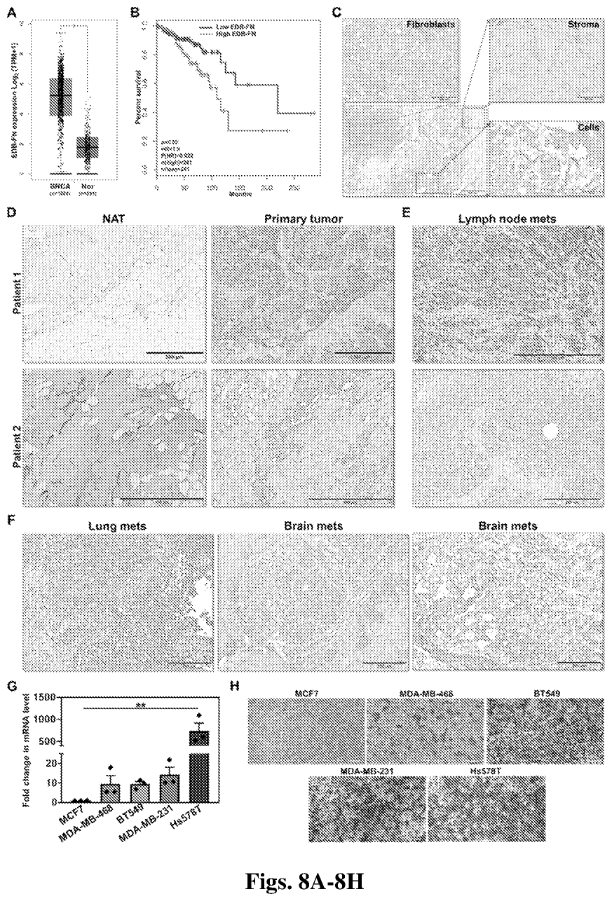

As a critical ECM component, FN1 is overexpressed in multiple cancer types. Han, Z., and Lu, Z. R. (2017), J Mater Chem B 5, 639-654. Here, the expression of its oncofetal isoform EDB-FN (transcript ID: ENST00000432072.6) in 1084 breast tumor and 291 normal breast samples from TCGA and GTEx databases was evaluated. Differential EDB-FN expression analysis and survival correlation data were derived from the web server GEPIA228. As shown in FIG. 8A, breast tumors demonstrated significant overexpression of EDB-FN [median 36.319 transcripts per million (TPM)], compared to normal breast tissues (median 2.410 TPM), translating to an extremely significant log₂ fold increase of 3.452 (adj p value=6.57e-69) in breast cancer over normal tissues. Furthermore, higher expression of EDB-FN was found to significantly correlate with poor overall survival (FIG. 8B) in the breast cancer patients, with a hazard ratio of 1.9 [p(HR)=0.022], demonstrating its prognostic value.

To gain insight into the location and patterns of EDB-FN expression in clinical samples, breast tumor specimens and normal adjacent tissues were stained with EDB-FN-specific G4 antibody. As shown in FIG. 8C, EDB-FN was abundantly expressed in cancer cell-associated fibroblasts (green), stroma and stromal fibroblasts (red), as well as the mitotic tumor cells (purple) of breast cancer. Further, the expression of EDB-FN was significantly higher in breast tumor tissues compared to the adjacent tissues (FIG. 8D). Besides primary tissues, EDB-FN was also highly expressed in lymph node (FIG. 8E), lung, and brain metastases (FIG. 8F), indicating that the oncofetal EDB-FN isoform is highly expressed in malignant breast phenotypes.

Next, the endogenous levels of EDB-FN mRNA were determined across a panel of cell lines representing the multiple molecular subtypes of breast cancer in correlation with their invasive properties. As shown in FIG. 8G-H, the least invasive hormone receptor-positive (HR⁺) MCF7 cells showed the lowest expression of EDB-FN. The more invasive triple-negative (HR⁻) breast cancer lines showed significant upregulation of EDB-FN levels, with approximately 9-10-fold increase in MDA-MB-468 and BT549, 14-fold increase in MDA-MB-231 cells, and over 700-fold increase in Hs578T cells. Taken together, these results demonstrate the potential of EDB-FN overexpression as a molecular marker of breast cancer.

Growth and Morphology Changes in 2D- and 3D-Cultured Breast Cancer Cells with TGF-β Treatment and Drug Resistance

To assess the changes in EDB-FN expression levels when breast cancer cells gain significant survival advantages, the two cell lines with the lowest EDB-FN expression and epithelial phenotype, namely MCF7 and MDA-MB-468 cells, were chosen. Two selective pressures were applied: 1) long-term treatment with TGF-β (5 ng/mL) to induce EMT30 to generate MCF7-TGFβ and MDA-MB-468-TGFβ cells and 2) acquired chemoresistance to Palbociclib, a cyclin-dependent kinase (CDK) inhibitor, and to Paclitaxel, an anti-microtubule agent, to generate MCF7-DR and MDA-MB-468-DR cells, respectively. Acquisition of drug resistance was confirmed by the significant overexpression of the drug resistance marker P-glycoprotein 1 or multidrug resistance protein (MDR1) in MCF-DR and MDA-MB-468-DR cells. The parent and derivative cell lines were characterized for their morphology, and molecular and functional phenotypes.

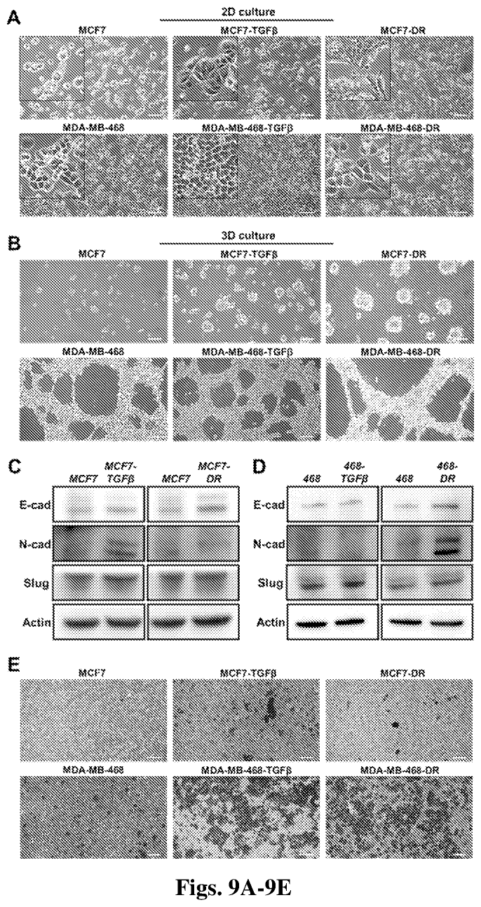

As shown in FIG. 9A, MCF7 cells demonstrated a typical epithelial morphology in 2D culture. Long-term treatment with TGF-β and development of resistance to Palbociclib resulted in morphological changes to a more mesenchymal phenotype, which was more pronounced in the MCF7-DR cells than in MCF7-TGFβ cells. On the other hand, the MDA-MB-468 cells did not exhibit overt changes in morphology with TGF-β treatment and development of resistance to Paclitaxel. However, the MDA-MB-468-TGFβ showed increased growth rate compared to the parent MDA-MB-468 cells.

In addition to 9D culture, the cells were grown in Matrigel to facilitate the establishment of a conducive ECM. As shown in FIG. 9B, in 9D culture, the low-risk HR* MCF7 cells showed negligible tumor spheroid formation while the more invasive MDA-MB-468 cells showed proliferative network formation. The MCF7-TGFβ and MCF7-DR cells formed tumor spheroids, unlike the parent MCF7 cells, while the MDA-MB-468-TGFβ and MDA-MB-468-DR cells formed similar proliferative networks as their parent counterparts. These results highlight the different properties of each cancer cell type and its distinct response to external mitogenic stimuli.

Increased Migration in Breast Cancer Cells with TGF-β Treatment and Drug Resistance

Next, we analyzed the molecular and functional changes in the TGF-β-treated and drug-resistant breast cancer cells. TGF-β is a potent inducer of EMT, a critical step towards initiation of metastasis. Similarly, the signaling programs of EMT and drug resistance are intricately related, where EMT-like molecular signature can antagonize chemotherapy in breast cancer. Huang, J., Li, H., and Ren, G. (2015), Int J Oncol 47, 840-848. Consequently, the expression of the common EMT markers, N-cadherin (N-cad), E-cadherin (E-cad), and Slug (invasion marker), was tested in the derivative cell lines. As shown in FIG. 9C, both MCF-TGFβ and MCF7-DR cells showed upregulated E-cad and N-cad expression, with only moderate increase in Slug expression, indicating that the MCF7 cells gain a partial EMT-like phenotype with TGF-β treatment and development of drug resistance. On the other hand, the MDA-MB-468-TGFβ cells showed no changes in E-cad and N-cad and a moderate increase in Slug levels while the MDA-MB-468-DR cells showed increased E-cad, N-cad, and Slug expression, compared to the parent cells (FIG. 9D). These results indicate that while both the MDA-MB-468-TGFβ and MDA-MB-468-DR cells overexpress the migratory protein Slug, the former did not undergo EMT with TGF-β treatment while the latter possibly gained a hybrid E-M phenotype with drug resistance.

The treated cell populations were analyzed for their ability to invade through a layer of matrigel coated in transwell inserts. As shown in FIG. 9E, both the TGF-β treatment and drug resistance conferred significant invasive advantage on the MCF7 and MDA-MB-468 cells, rendering them more motile than their parent counterparts, as seen by the increased number of crystal violet-stained migrated cells. Indeed, recent studies have revealed that the partial or hybrid E-M phenotype is attributed to the tumor cell plasticity and is extremely favorable for metastatic dissemination. Saitoh, M. (2018), J Biochem 164, 257-264.

Increased EDB-FN Expression in Breast Cancer Cells with TGF-β Treatment and Drug Resistance

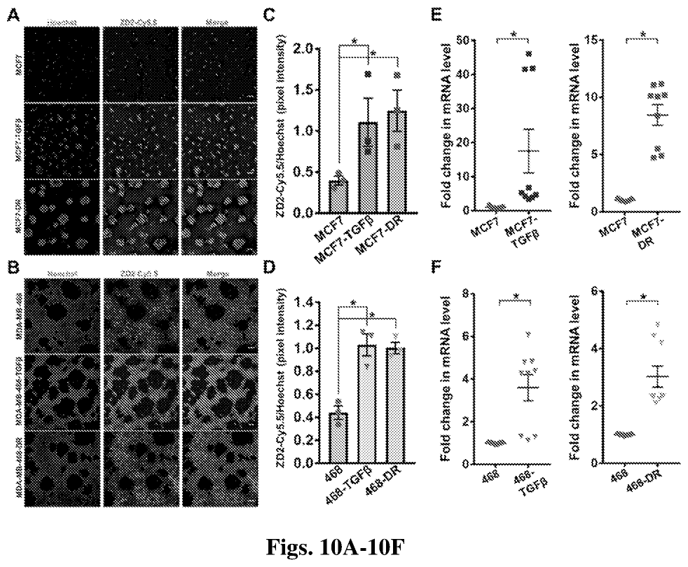

The potential role of EDB-FN as a molecular marker for aggressiveness of breast cancer cells was then determined in TGF-β-treated and drug-resistant MCF7 and MDA-MB-468 cells. The expression of EDB-FN in 3D-cultured cells was analyzed using fluorescent-labeled EDB-FN-specific peptide ZD2-Cy5.521. As shown in FIG. 3A-B, endogenous EDB-FN expression in MDA-MB-468 cells is higher than that in the MCF7 cells, consistent with the mRNA levels in FIG. 8G, and their invasive ability in FIGS. 8H and 9E. Treatment with TGF-β and acquired drug resistance led to a significant increase in EDB-FN expression in the 3D-cultured MCF7 and MDA-MB-468 cells, reflected in the increased ZD2-Cy5.5 binding in FIG. 10A-B and Hoechst-normalized ZD2-Cy5.5 pixel intensities in FIG. 10C-D. The peptide binding results were also corroborated by qRT-PCR analysis, which showed over 17-fold and 8.5-fold increase in EDB-FN expression in MCF7-TGFβ and MCF7-DR cells (FIG. 10E) and over 3-fold increase in MDA-MB-468-TGFβ and MDA-MB-468-DR cells (FIG. 10F), over their respective counterparts.

The EDB-FN-specific binding of the ZD2-Cy5.5 probe was confirmed by EDB-FN knockdown experiments in MDA-MB-468-DR cells, where ECO/siEDB nanoparticle treatment abrogated the ZD2-Cy5.5 binding (S FIG. 9A) and silenced EDB-FN mRNA expression by over 70% (S FIG. 9B). These results indicate that irrespective of the acquisition of hybrid E-M-like changes, increased invasiveness in breast cancer cells results in significant upregulation of EDB-FN. Thus, EDB-FN overexpression is associated with invasive breast cancer cells and with the breast cancer cells that evolve from low-risk ones.

### Therapeutic Ablation of AKT in Invasive Breast Cancer Cells Decreases Invasion and EDB-FN Expression

Non-invasive therapeutic monitoring of tumor response to oncostatic drugs is crucial to facilitate decision making and timely interventions6. To test whether EDB-FN is a therapy-predictive marker and if its expression correlates with changes in the invasive potential of breast cancer cells, the TGF-β-treated and drug-resistant MCF7 and MDA-MB-468 cells were treated with MK2206-HCl, a highly specific pan-AKT inhibitor proven to suppress PI3K/AKT signaling-induced tumor cell proliferation. The PI3K/AKT signaling is a major signal transduction cascade implicated in the progression, metastasis, and drug resistance of multiple cancers.

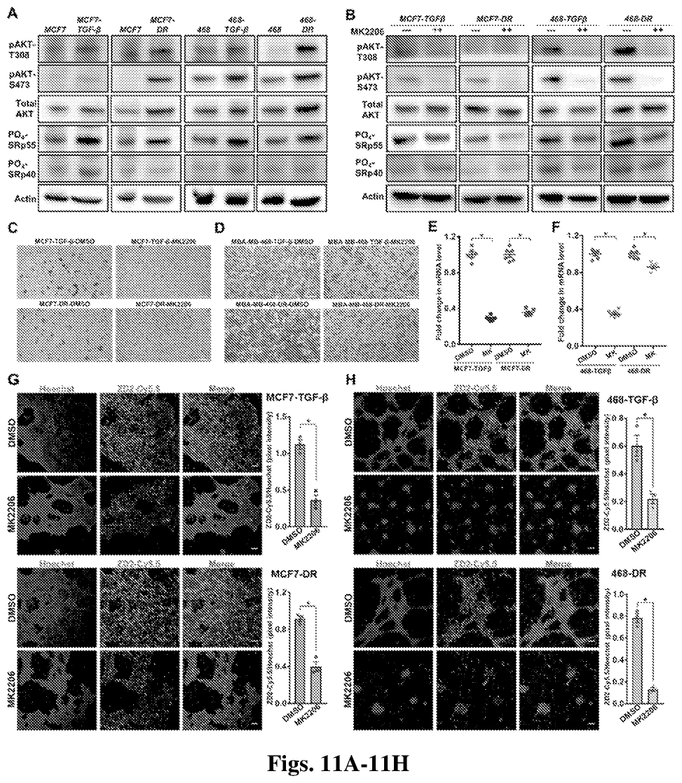

The upregulation of the mitogenic AKT signaling axis in the aggressive TGF-β-treated and drug-resistant MCF7 and MDA-MB-468 cell populations was first confirmed by testing for the levels of phosphorylated AKT (T308 and S473) and total AKT (FIG. 11A). Both the phospho-AKT-T308 and phospho-AKT-S473 levels were strongly upregulated in the TGF-β-treated and drug-resistant MCF7 and MDA-MB-468 cells, compared to their respective parent cells. Total AKT was also upregulated in both the cell lines with drug resistance, and to a lesser extent with TGF-β treatment. In addition, previous studies have implicated the role of phosphoAKT-SRp40 pathway in the alternative splicing-mediated regulation of EDB-FN expression. Bordeleau et al., (2015), Proc Natl Acad Sci USA 112, 8314-8319. To determine the mechanism of EDB-FN upregulation by TGF-β and drug resistance, we examined the expression of phosphorylated SR proteins (SRp55 and SRp40). The more invasive, MCF7-TGFβ, MCF7-DR, MDA-MB-468-TGFβ, and MDA-MB-468-DR cells were found to upregulate the expression of SRp55, while only MCF7-TGFβ and MDA-MB-468-TGFβ upregulated SRp40, compared to their respective parent counterparts. Treatment of the invasive cell derivatives with MK2206-HCl resulted in robust inhibition of phospho-AKT (T308 and S473), as shown in FIG. 4B. The upregulation of phosphorylated SRp55 was also diminished in the four cell lines with MK2206-HCl treatment, suggesting that the EDB-FN upregulation in these invasive cells could be controlled, at least in part, through the phosphoAKT-SRp55 signaling pathway (FIG. 11B).

Functionally, MK2206-HCl-mediated phospho-AKT depletion reduced the invasive potential of the TGFβ-treated and drug-resistant cell derivatives (FIG. 11C-D). This was also accompanied by a significant decrease in the expression of EDB-FN in the EDB-FN-overexpressing TGF-β-treated and drug-resistant MCF7 and MDA-MB-468 cells, demonstrated by the significant decrease in the mRNA levels (FIG. 11E-F) and reduced ZD2-Cy5.5 staining intensities in 3D cultures (FIG. 11G-H), compared to the respective DMSO-treated controls. Specifically, in MDA-MB-468-DR cells, although EDB-FN mRNA showed a moderate decrease (about 10-15%), the in situ EDB-FN expressed by 3D spheroids reduced significantly, possibly contributing to the robust decrease in invasion by MK2206-HCl treatment. These results demonstrate a positive association between EDB-FN and invasiveness of breast cancer cell lines, and potential correlation of altered EDB-FN expression with response to therapeutic interventions.

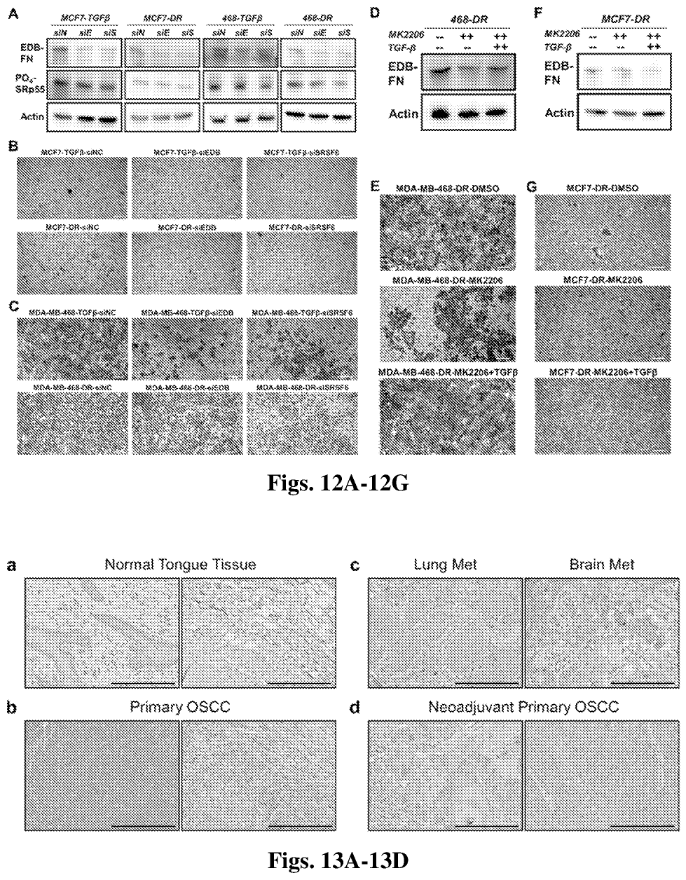

To further explore the link between the SRp55-EDB-FN pathway and invasiveness, the TGF-β-treated and drug-resistant MCF7 and MDA-MB-468 cells were transfected with siEDB-FN or siSRSF6-bearing nanoparticles to evaluate the loss-of-function effects on the motility of the cells. As shown in FIG. 12A, compared to NC (siN), siEDB-FN (siE) and siSRSF6 (siS) treatments resulted in reduced expression of EDB-FN and SRp55, respectively, in the 4 derivative cell lines. Further, downregulation of SRp55 (siSRSF6) also resulted in downregulation of EDB-FN levels, validating the role of SRp55 in the regulation of EDB-FN expression. Moreover, the reduced levels of both EDB-FN and SRp55 were associated with reduced invasive potential of the TGF-β-treated and drug-resistant MCF7 and MDA-MB-468 cells (FIG. 12B-C), suggesting a direct or indirect role of EDB-FN in regulating the invasive patterns of breast cancer cells. Treatment of MK2206-HCl-treated MDA-MB-468-DR cells with TGF-β was found to rescue the EDB-FN overexpression in these cells (FIG. 12D), which was also accompanied with an increase in invasion (FIG. 12E), suggesting that the pAKT-mediated invasion in MDA-MB-468-DR cells may occur, at least in part, through EDB-FN. On the other hand, while treatment of MK2206-HCl reduced the EDB-FN expression (FIG. 12F) and invasion (FIG. 12G) of MCF7-DR cells, addition of TGF-β did not rescue their EDB-FN expression or invasive potential. It is possible that the pAKT-depletion alters the response of the ER⁺ MCF7-DR cells to TGF-β, given that TGF-β and AKT signaling pathways are tightly intertwined in cancer and blocking pAKT has been shown to repress TGF-β-induced pro-neoplastic responses. Nevertheless, the results demonstrate a strong positive association between the patterns of EDB-FN expression and invasive potential of cells, indicating the potential of EDB-FN as a molecular marker of invasion in breast cancer cells.

### Discussion

Numerous blood biomarkers including CA 15.3, carcinoembryonic antigen (CEA), CA125 and imaging modalities like ultrasound, mammography, MRI, PET, and CT are routinely used to detect primary breast tumor disease and recurrence and to assess therapeutic response. Bayo et al., (2018) Clin Transl Oncol 20, 467-475. However, they are limited in their ability to differentially diagnose and risk-stratify the disease, with high rates of false positive diagnoses, underscoring the need for specific markers to accurately detect highly invasive and metastatic breast tumors, and to distinguish them from low-risk indolent ones. Moreover, breast tumors frequently exhibit intrinsic or acquired resistance to chemotherapy and targeted drugs. In the absence of suitable molecular markers, active surveillance and monitoring of the efficacy of chemotherapeutic interventions and timely detection of the emergence of resistant phenotypes forms another obstacle to patient treatment.

To address these concerns, this study investigated the dynamic changes in the ECM oncoprotein EDB-FN in conjunction with the dynamic changes in the invasive potential of breast cancer cells. We found that invasive cells that evolve from low-risk cancer cells can exhibit partial E-M phenotypes and overexpress EDB-FN; conversely, impeding the invasive abilities of these high-risk cancer cells with a targeted drug abolishes their EDB-FN overexpression, demonstrating a direct correlation between EDB-FN levels and the invasiveness of breast cancer cells. Moreover, to our knowledge, this is the first study to report that EDB-FN is upregulated with development of drug resistance in breast cancer cells.

Between the two different breast cancer lines used in this work, the endogenous EDB-FN level in the least aggressive HR* MCF7 cells is significantly lower than that in the more aggressive triple-negative MDA-MB-468 cells, despite both lines exhibiting an epithelial phenotype. Induction of drug resistance and long-term TGF-β treatment led to distinct changes in the molecular phenotypes of the two cell lines, possibly through distinct signaling mechanisms. The emergent invasive populations became more aggressive than their parent cells, with a pre-metastatic hybrid E-M phenotype and increased phospho-AKT signaling. Indeed, recent studies suggest that the existence of subpopulations of cancer cells along different positions on the E-M spectrum confers profound plasticity on the tumors, promoting their progression, metastasis, and stemness. It is also likely that each of these cell lines acquired their survival advantages through additional here-to-fore unstudied mechanisms. Nevertheless, all of the cells with acquired invasiveness presented elevated EDB-FN expression irrespective of the signaling mechanisms. Conversely, when the aggressive cells were treated with targeted therapy, their invasive potential diminished with a concomitant reduction in EDB-FN expression. The invasiveness was rescued only when subsequent TGF-β treatment upregulated the EDB-FN expression in MDA-MB-468-DR cells but not in MCF7-DR cells, indicating the role of EDB-FN as a therapy-predictive marker for active surveillance and monitoring of breast cancer.

The precise mechanism of EDB-FN upregulation in invasive cells remains an enigma. At the genetic level, EDB-FN is generated by alternative splicing event, resulting in the inclusion of the EDB exon in the FN1 transcript, a process controlled by SR (Ser- and Arg-rich) proteins of the splicing regulator family. Since alternative splicing is indispensable for the formation of the EDB-FN isoform, the participation of the SR proteins in this process is inevitable. However, there is limited research on the underlying mechanism of the preferential and differential inclusion of the EDB exon during neoplastic transformation. Previous studies show that increased tissue stiffness directly upregulates PI3K/AKT-mediated SRp40 phosphorylation, enhancing exon inclusion and EDB-FN secretion by breast cancer cells. Bordeleau et al., (2015), Proc Natl Acad Sci USA 112, 8314-8319. In this work, development of drug resistance and TGF-β treatment in MCF7 and MDA-MB-468 cells consistently upregulated SRp55 phosphorylation, in addition to increased phospho-AKT. Knockdown of SRp55, directly using RNAi or indirectly using MK2206-HCl, significantly reduced their EDB-FN expression as well as invasive potential. Given that SRp55 is commonly mutated in breast and colorectal cancers and influences the alternative splicing patterns of several tumor-associated genes like KIT, CD44, and FGFR147, it is not surprising that SRp55 depletion decreased the invasion of breast cancer cells. However, this is the first study to report decreased EDB-FN expression as a consequence of SRp55 depletion. How SRp55, and the other SR proteins, acts in conjunction with their antagonistic hnRNPs in the spliceosome, to regulate the complex alternative splicing processes in response to various intrinsic and extrinsic stimuli remains to be explored. Additionally, MK2206-HCl treatment showed highly specific knockdown of phospho-AKT and consequent downregulation of SRp40/SRp55 levels in a cell-specific manner. While the MK2206-HCl treatment significantly reduced EDB-FN expression and invasion, it did not completely abrogate them, suggesting the compensatory activation of other mitogenic proteins (like AKT3) or the EDA-FN isoform, which is also involved in tumorigenesis. Han, Z., and Lu, Z. R. (2017), J Mater Chem B 5, 639-654. Given the complex composition and molecular signaling in breast malignancies, it would be interesting to evaluate the distinct spatial and temporal changes in EDB-FN expression and function following different drug treatments.

EDB-FN is overexpressed in multiple types of cancer, including breast, oral, lung, and prostate. Khan et al., (2005), Exp Lung Res 31, 701-711; Albrecht et al., (1999), Histochem Cell Biol 112, 51-61. Originally thought to be secreted only by cancer-associated fibroblasts (CAFs) and endothelial cells, EDB-FN is now known to be abundantly produced by tumor cells, especially invasive tumor cells. EDB-FN is upregulated during embryogenesis, temporally activated during wound healing, tissue repair, and angiogenesis, but mostly absent from healthy adult tissues. White et al., (2008), J Pathol 216, 1-14. Indeed, immunohistochemical analysis in our study showed robust EDB-FN expression in the mitotic breast tumor cells, diffuse staining in the stroma, stromal fibroblasts, and fibroblasts interspersed between cancer cells, in primary tumor specimens as well as metastatic sites, with negligible expression in normal breast tissues, suggesting complex structural and functional roles of EDB-FN in the progression of malignancies. In TCGA/GTEx samples, EDB-FN is significantly overexpressed in breast cancer and is negatively correlated with patient survival. Additionally, by virtue of its extracellular location and ready accessibility, EDB-FN has emerged as an attractive target for designing new diagnostic and therapeutic regimens. Previous studies have already demonstrated the potential of antibody-mediated EDB-FN targeting (using L19, BC-1) for angiogenesis, inflammation, and cancer stem cell therapy. Mariani et al., (1997), Cancer 80, 2378-2384. EDB-FN-specific peptides, such as ZD2 and APTEDB, are advantageous for oncogenic ECM targeting, by virtue of their small size, low immunogenicity, and high tissue penetration ability. Zahnd et al., (2010), Cancer Res 70, 1595-1605. The specificity and superior binding of the ZD2 probe for EDB-FN has direct translational implications. We have successfully demonstrated differential diagnosis of non-invasive and invasive breast and prostate cancer xenografts in mouse models using ZD2-targeted MRI contrast agents. Han et al., (2017), Bioconjug Chem 28, 1031-1040. The results of this study open up new avenues for determining the potential of EDB-FN as a molecular biomarker for molecular imaging-based detection, risk-stratification, active surveillance, and monitoring of breast cancers and tracking their evolution as the disease progresses with and without chemotherapy.

In summary, this research shows that EDB-FN expression is associated with highly invasive breast cancer and with low-risk cells that evolve into high-risk ones. This correlation holds true despite cancer cell plasticity, and dynamic changes occurring in the invasive properties of breast cancer cells lead to corresponding changes in the EDB-FN expression levels. In addition to demonstrating for the first time that acquired drug resistance upregulates EDB-FN, we have shown that this enhanced expression, in part, occurs through the phosphoAKT-SRp55 signaling pathway. These observations indicate that EDB-FN is a promising molecular marker for monitoring the progression of breast cancer, in the context of diagnostic imaging and therapeutic interventions.

### Materials and Methods

### Cell Lines and Culture

MCF7, MDA-MB-231, BT549, and Hs578T cells were purchased from ATCC (Manassas, VA). MCF7-DR cells (resistant to 500 nM Palbociclib), MDA-MB-468, and MDA-MB-468-DR (resistant to 100 nM Paclitaxel) cells were a kind gift from Dr. Ruth Keri (CWRU, Cleveland, OH). MCF7-TGF-β and MDA-MB-468-TGF-β cells were obtained by treating the parent lines with 5 ng/mL TGF-β (RnD Systems, Minneapolis, MN) for at least 7-10 days. The breast cancer lines were cultured in Dulbecco's Modified Eagle's Medium (DMEM) supplemented with 10% fetal bovine serum (FBS), and 100 Units/mL Penicillin/Streptomycin (P/S). MCF7, MCF7-DR, and Hs578T cells were additionally supplemented with 0.01 mg/mL human insulin (Sigma-Aldrich, St. Louis, MO). All the cells were grown at 37° C. and 5% CO₂. The cell lines were tested for the absence of Mycoplasma using the MycoAlert™ Mycoplasma Detection Kit (Lonza, Allendale, NJ). Cell lines were also authenticated by Genetica DNA Laboratories (Burlington, NC).

### MK2206-HCl Treatment

The invasive TGF-β-treated and drug-resistant MCF7 and MDA-MB-468 populations were treated with MK2206-HCl, a pan-AKT inhibitor, purchased from SelleckChem (Boston, MA). For this treatment, 8×10⁵ cells were plated on 6-well plates. After 24 h of attachment, the cells were treated with MK2206-HCl for 2 days (2 μM dose for MCF7 cells and 4 μM dose for MDA-MB-468 cells). Cells treated with equivalent volume of DMSO were used as controls. After treatment, the cells were counted and an equal number of cells was plated on Matrigel and in transwell inserts for the invasion and 3D growth assays, as described in the relevant sections. The treated and non-treated cells were similarly counted and harvested for protein and RNA extraction for western blotting and qRT-PCR, respectively.

### Gene Data Analyses

Gene expression analysis and survival curve data was derived from the web server GEPIA228, which provided breast tumor-normal comparison and overall survival curve of the EDB-FN transcript (ENST00000432072.6) from TCGA (tumor and normal) and GTEx (normal) databases (1084 BRCA and 291 normal tissue samples). The expression data are first log 2(TPM+1) transformed for differential analysis and the log₂FC is defined as median(Tumor)−median(Normal). GEPIA2 uses Log-rank test, or the Mantel-Cox test, for hypothesis test for survival analysis; Cox PH Model for hazards ratio calculation, and ANOVA or LIMMA for differential gene expression analysis.

### Human Tissue Specimens and IHC

Human tissue specimens (breast primary tumors, normal adjacent tissue, and metastases in lung, lymph node, and brain) were obtained from the Human Tissue Procurement Facility at Case Western Reserve University (CWRU). All the samples were de-identified and de-classified. Tissue sectioning and IHC services were provided by the Tissue Resources Core Facility of the Comprehensive Cancer Center of CWRU and University Hospitals of Cleveland (grant P30 CA43703). IHC for EDB-FN was performed using 1:100 dilution of anti-EDB-FN antibody G4 clone (Absolute Antibody, UK) after a short antigen retrieval step (30 s at 125° C. in citrate buffer). The slides were reviewed by a certified pathologist. The images were acquired on Bx61VS slide scanner microscope (Olympus, Waltham, MA) with 40× objective lens and were processed in OlyVIA software.

qRT-PCR

Total RNA was extracted from cells and tissues using the RNeasy Plus Mini Kit (Qiagen, Germantown, MD), according to manufacturer's instructions. RNA concentration was determined and 1 μg of RNA was used to set up reverse transcription using the miScript II RT Kit (Qiagen) and qPCR was performed using the SyBr Green PCR Master Mix (Applied Biosystems, CA). Gene expression was analyzed by the 2-ΔΔCt method with 18S or R-actin expression as the control.

### 3D Tumor Spheroid Growth and ZD2-Cy5.5 Staining

For the Matrigel growth assay, 5×10⁵ breast cancer cells were suspended in 5% Matrigel-containing media and plated on a thick layer of Corning™ Matrigel™ Membrane Matrix. The ability of the cells to form tumor spheroids in the 3D Matrix was monitored and photographed for up to 5 days using the Moticam T2 camera with 10× objective lens. After 2-4 days, the cells were stained with Hoechst-33342 (5 μg/mL) and ZD2-Cy5.5 (100 nM) for 30 min at 37° C. After 3 washes of PBS, fresh media was added and the cells were imaged on Olympus FV1000 confocal laser scanning microscope (10× objective lens) to obtain Z-stack images. Image analysis was done in FIJI software and ZD2 peptide binding to EDB-FN was quantified as the ratio of the pixel intensities of ZD2-Cy5.5 to that of Hoechst 33342.

### Western Blot

Total cellular protein was extracted as previously described. Vaidya et al., (2019) Bioconjug Chem 30, 907-919. Protein concentration was determined by Lowry assay and equal concentration of protein extracts (40 μg) was loaded on SDS-PAGE gels, transferred onto nitrocellulose membrane and immunoblotted with primary antibodies overnight. Anti-EDB-FN antibody (G4 clone) was used at 1:1000 dilution. The following primary antibodies (1:1000 dilution) were purchased from Cell Signaling Technology (Danvers, MA): anti-E-cadherin (Cat #3195), anti-Slug (Cat #9585), anti-phospho-T308-AKT (Cat #13038), anti-phospho-S473-AKT (Cat #4060), anti-pan-AKT (Cat #4691), anti-MDR1 (Cat #12683S); and anti-Histone H3 (Cat #4499) and anti-R-actin (Cat #4970) as loading controls. The anti-Phosphoepitope SR proteins (Cat #MABE50; clone 1H4) and anti-SRp40 (Cat #06-1365) antibodies were purchased from Millipore Sigma (Temecula, CA) and used at 1:500 dilution. Anti-N-Cadherin antibody (Cat #76057) was purchased from Abcam (Cambridge, MA) and used at 1:500 dilution.

### Transwell Assay

For the invasion assay, breast cancer cells were starved in serum-depleted media overnight. The next day, 1-2×10⁵ cells were plated in transwell inserts (VWR, Radnor, PA) coated with 0.3 mg/mL Corning™ Matrigel™ Membrane Matrix (Corning, NY). After 1-2 days, the inserts were swabbed with Q-tips to remove the plated cells. The invading cells on the bottom of the inserts were fixed with 4% paraformaldehyde followed by staining with 0.1% crystal violet for 20 min. Excess stain was washed under tap water and images of the purple migrated cells were taken using the Moticam T2 camera with 10× objective lens.

### Gene Knockdown Assays

ECO/siRNA nanoparticles were formulated as previously described. Gujrati et al., (2016), Adv Healthc Mater 5, 2882-2895. Briefly, the amino lipid ECO (5 mM stock in ethanol) was mixed with siLuc (as negative control NC) or siEDB-FN or siSRSF6 at a final siRNA concentration of 100 nM and N/P=10 for 30 min to enable self-assembly formation of ECO/siNC, ECO/siEDB, or ECO/siSRSF6 nanoparticles, respectively. For transfections, the nanoparticle formulation was mixed with culture media and added on to the cells. After 24-48 h, the cells were harvested for transwell assays or added onto Matrigel for staining with ZD2-Cy5.5 as described above. The siRNA duplexes were purchased from purchased from Dharmacon (Lafayette, CO).

### Statistical Analyses

All the experiments were independently performed in triplicates (n=3), unless otherwise stated. Bar graphs are represented as mean±s.e.m with individual dots denoting the replicates. Statistical analysis was performed using Graphpad Prism version 7.03. Data between two groups was compared using unpaired Student's t-test and Mann-Whitney U test. EDB-FN mRNA data between breast cancer cell lines was analyzed using Kruskal-Wallis test. p<0.05 was considered to be statistically significant.

### Example 3: Preclinical Assessment of the Effectiveness of Magnetic Resonance Molecular Imaging of Extradomain-B Fibronectin for Detection and Characterization of Oral Cancer

Oral squamous cell carcinoma (OSCC) is the most common malignant neoplasm of the oral cavity, representing more than 90% of oral malignancies. Despite improvements in therapeutic options, the 5-year survival rate of OSCC patients remains around 50% with little improvement. This poor prognosis stems from late-stage diagnosis, local invasion and metastasis, recurrence post-resection, and lack of targetable biomarkers for early and accurate detection. Over 60% of patients are diagnosed with late-stage tumors, whose overall survival rate plummets to as low as 20%. Conventional histological grading schemes do not reliably predict patient outcomes, necessitating the development of enhanced OSCC diagnostic methods to improve clinical outcomes.

Routine work-up for OSCC patients includes physical examination and diagnostic imaging to better delineate disease margins. Most patients have locally or regionally aggressive disease that is difficult to diagnose, and micrometastases to local lymph nodes highly correlate with adverse outcomes. While the oral cavity can be physically examined, routine examinations do not consistently identify all biologically relevant precursor lesions, and adjuvant screening techniques lack the appropriate sensitivity and specificity to justify widespread use. Common diagnostic imaging methods for OSCC include computed tomography (CT) and magnetic resonance imaging (MRI). Although both provide valuable diagnostic information, MRI offers superior soft tissue contrast, enabling more precise delineation of primary tumor boundaries, local invasion, and detection of metastases. Consequently, MRI is often used to plan the scope of surgical resection, subsequent tissue reconstruction, and treatment monitoring for therapeutic efficacy and recurrence. Palasz et al., (2017), Pol J Radiol 82:193-202. The most commonly used MRI contrast agents are gadolinium-based contrast agents (GBCAs). However, current GBCAs are untargeted, providing non-specific contrast enhancement with no ability for disease characterization. Zhou Z, Lu Z R (2013), Wiley Interdiscip Rev Nanomed Nanobiotechnol 5:1-18. There is therefore an unmet clinical need to develop targeted GBCAs that enable precision imaging of OSCC for accurate detection, delineation, and characterization to enhance diagnosis and guide timely precision treatment.

Tumor extracellular matrix (ECM) proteins closely interact with tumor cells and mediate many biological processes associated with tumor progression. Upregulated ECM fibronectin is associated with tumor invasion, metastasis, and therapy resistance. Extradomain-B fibronectin (EDB-FN), a fibronectin splice-variant involved in neovascularization, is also upregulated in many aggressive cancers, including pancreatic, breast, and oral cancers, with little expression in normal adult tissues. Han Z, Lu ZR (2017), J Mater Chem B 5:639-654. The presence of EDB-FN in epithelial tumor cells indicates their inherent ability to produce the protein in conjunction with EDB-FN produced by stromal cells. Recent studies show differential expression of EDB-FN in prostate and breast cancers positively correlating with tumor aggressiveness. Ayat et al. (2018), ACS Med Chem Lett 9:730-735; Ayat et al. (2019), Front Oncol 9:1351. In OSCC, EDB-FN exhibits variable expression in upwards of 100% of primary tumors and 63-96% of cervical metastases, suggesting its potential for diagnostic imaging of aggressive OSCC. Birchler et al. (2003), Laryngoscope 113:1231-1237

We developed a small peptide, ZD2, to selectively target EDB-FN in the tumor microenvironment. Han et al., (2015), Bioconjug Chem 26:830-838. Conjugation of ZD2 to the clinical GBCA gadoteridol yielded a novel EDB-FN-targeting contrast agent, ZD2-Gd(HP-DO3A), that enabled magnetic resonance molecular imaging (MRMI) of the tumor microenvironment. Han et al., (2017), Bioconjug Chem 28:1031-1040. Further optimization of ZD2-Gd(HP-DO3A) produced a more efficient targeted contrast agent ZD2-N3-Gd(HP-DO3A) (MT218) with an improved T1 relaxivity. MRMI with the targeted agents differentially enhanced aggressive forms of prostate and triple-negative breast cancers. A dosing study demonstrated that MT218 provides comparable, if not greater, contrast enhancement in triple-negative breast cancer even at 20% of the recommended clinical dose (0.1 mmol/kg) for Gd(HP-DO3A) or gadoteridol. 15. Ayat et al., (2018), ACS Med Chem Lett 9:730-735. Here, we demonstrate the effectiveness of MT218 at reduced dose for diagnostic imaging of EDB-FN-expressing aggressive OSCC using MRMI.

### Methods

### Cell Culture

Human OSCC cell lines CAL27 and SCC4 were purchased from American Type Culture Collection (ATCC, Manassas, VA, USA). OSCC cell line HSC3 was purchased from the Japanese Collection of Research Bioresources Cell Bank (Ibaraki City, Japan) via Sekisui XenoTech (Kansas City, KS, USA). CAL27 and HSC3 were cultured in Dulbecco's Modified Eagle's Medium (DMEM, ATCC) supplemented with 10% fetal bovine serum (FBS, Corning Inc., Corning, NY, USA) and 1% penicillin-streptomycin (PS, Thermo Fisher Scientific, Waltham, MA, USA). SCC4 was cultured in DMEM supplemented with 10% FBS, 1% PS, and 400 ng/mL hydrocortisone (Sigma-Aldrich, St. Louis, MO, USA). Stable green fluorescent protein (GFP)- and firefly luciferase-expressing cell lines were generated by transfecting cells with CMV-Luciferase-2A-GFP lentivirus (Amsbio, Cambridge, MA, USA) followed by fluorescence-activated cell sorting for GFP expression. CAL27-GFP, SCC4-GFP, and HSC3-GFP cell lines were cultured in the complete medium of the respective parent cell line. All cells were incubated at 37° C. in 5% CO₂.

### Semi-Quantitative Real-Time PCR

RNA was extracted from cell pellets with an RNeasy Kit (Qiagen, Hilden, Germany). RNA samples were reverse-transcribed into cDNA using a miScript II Reverse Transcription Kit (Qiagen). Semi-quantitative real-time PCR was conducted using a SYBR Green Master Mix (Life Technologies, Carlsbad, CA, USA) according to the manufacturer's recommendations. Relative mRNA levels were calculated using the standard 2-ΔΔCT method, using 18S expression as the control gene. cDNA synthesis and qRT-PCR were conducted with the BioRad CFX Connect Real-Time PCR Detection System (Bio-Rad Laboratories, Hercules, CA, USA). Primers were purchased from Integrated DNA Technologies (IDT, Coralville, IA, USA).

### Western Blot

Cell lysates were prepared from cell pellets with 2× Laemmli sample buffer (Bio-Rad Laboratories) and a protease inhibitor cocktail (Roche Holding AG, Basel, Switzerland) according to the manufacturer's recommendations. Lysates were boiled and centrifuged at 4° C. and 15,000 rpm for 15 minutes. The supernatants were collected, and protein concentration was measured via Lowry assay (Bio-Rad Laboratories). Protein extracts (50 μg) were resolved using SDS-PAGE and transferred onto nitrocellulose membranes (Cell Signaling Technology, Danvers, MA, USA) under ice. SDS-PAGE and transfer were conducted using the Bio-Rad Mini PROTEAN Tetra System (Bio-Rad Laboratories). Membranes were blocked with 5% milk, washed, and incubated with anti-EDB-FN (G4, Absolute Antibody, Oxford, UK) and anti-R-actin (CST) primary antibodies (1:2000) in 5% bovine serum albumin (BSA, Sigma-Aldrich) on ice overnight. Membranes were washed and incubated with horseradish peroxidase-conjugated anti-mouse and anti-rabbit IgG secondary antibodies (1:1000, CST) in 5% BSA for 1 hour at room temperature. After washing, membranes were activated with SignalFire Plus ECL Reagent (CST) and imaged with the BioRad Molecular Imager ChemiDoc XRS+ System (Bio-Rad Laboratories).

### Spheroid Formation and Peptide Binding Studies

Coverslip plates (4-well, Ibidi GmbH, Martinsried, Germany) were coated with Cultrex Basement Membrane Matrix (350 μL, Trevigen, Gaithersburg, MA, USA). For spheroid formation, 2×10⁵ OSCC cells were seeded into prepared wells, incubated for 2 days, and photographed with the Moticam T2 camera (Motic, Hong Kong, China). For peptide binding, 1×10⁵ cells were seeded into prepared wells and incubated for 2 days. Spheroids were stained with ZD2-Cy5.5 (125 nM), synthesized as previously described, and Hoechst (1:2000 dilution) (Invitrogen, Carlsbad, CA, USA) dyes for 15 minutes, washed 3× with DPBS (Thermo Fisher Scientific), and imaged with confocal laser scanning microscopy using the Olympus FV1000 system (Olympus Life Science, Tokyo, Japan).

Confocal fluorescence images were analyzed using FIJI. For quantification, images were thresholded to generate ROIs of both stains. For spheroid size, the average size of Hoechst ROIs was calculated. For staining intensity, the average signal intensities of ZD2-Cy5.5 and Hoechst ROIs were calculated, along with the ratio between the average intensities.

### Transwell Migration and Invasion Assays

OSCC cells were starved overnight, seeded in serum-free medium (1×10⁵ cells) into Transwell ThinCert Inserts (Greiner Bio-One, Kremsmunster, Austria) coated with (invasion) or without (migration) 100 μL of 1.0 mg/mL Matrigel (Corning), and placed above complete medium. Inserts for migration and invasion were incubated for 1 and 2 days, respectively. Cells on the underside of inserts were fixed with 10% formalin, stained with 0.1% crystal violet, and imaged with the Moticam T2 camera.

### Animal Models

Animal experiments were performed under an animal protocol approved by the Institutional Animal Care and Use Committee (IACUC) at Case Western Reserve University (CWRU, Cleveland, OH, USA). Female 4-week-old athymic nude mice purchased from the Jackson Laboratory (Bar Harbor, ME, USA) were housed in the Animal Core Facility at CWRU. Anesthetized mice were subcutaneously inoculated in the right flank with 4×10⁶ GFP/luciferase-expressing OSCC cells (n=5 per cell line) in 100 μL of a 1:1 mixture of Matrigel HC (Corning) and DPBS. Tumor size was monitored with caliper measurements. After 4 weeks of growth, tumors were sufficiently large for MRI experiments.

### MR Molecular Imaging

MRI experiments were performed with a 3T MRS-3000 system (MR Solutions, Surrey, UK). Tumor-bearing mice were imaged before and at 10, 20, and 30 minutes post-injection of the targeted contrast agent, MT218 (Molecular Theranostics, LLC, Cleveland, OH, USA), at a dose of 0.04 mmol/kg using a T1-weighted fast spin-echo axial sequence with respiratory gating (TR=305 ms, TE=11 ms, FOV=40×40 mm, slice thickness=1 mm, number of slices=15, Nav=2, matrix=256×248). This protocol was repeated on the same mice 2 days later using the untargeted clinical agent gadoteridol at the clinical dose of 0.10 mmol/kg.

Images were exported in DICOM format and analyzed using FIJI. Contrast-to-noise ratio (CNR) for each time point was calculated as the difference between the mean tumor intensity and the mean muscle intensity, divided by the standard deviation of noise.

### Xenograft Histological Analysis

Mice were euthanized according to IACUC guidelines. Xenograft tumors were fixed in 10% formalin, paraffin-embedded, and sectioned in 5 μm slices onto coverslip slides. Tissues underwent routine H&E staining for tissue morphology and immunohistochemical staining using the EDB-FN-specific antibody, G4, at a 1:100 dilution. Antigen retrieval was performed prior to immunohistochemistry using citrate buffer in a pressure cooker at 125° C. for 30 seconds. Tissue preparation and staining was conducted by the Tissue Resources Core at CWRU.

### Human Tissue Procurement and Histological Analysis

Human normal tongue and OSCC specimens were obtained in 5 μm slices on coverslip slides from the Human Tissue Procurement Facility at CWRU. Tissue samples were fully de-identified according to a non-human subject research protocol approved by the Institutional Review Board at CWRU. Immunohistochemical staining for EDB-FN was conducted by the Tissue Resources Core as described previously.

### Statistical Analysis

All experiments were independently replicated three times unless otherwise stated. Statistical significance between two groups was calculated using an unpaired T-Test. Statistical significance between more than two groups with one and two independent variables was calculated using one-way and two-way ANOVA, respectively, followed by Fisher's least significant difference post-hoc tests. P-values less than 0.05 were considered statistically significant. All data is presented as mean±standard error.

### Results

### EDB-FN Expression in Human Tissues

To evaluate the clinical relevance of targeting EDB-FN for OSCC in patients, EDB-FN expression in human tissue samples was evaluated by immunohistochemistry with an EDB-FN specific G4 monoclonal antibody. While normal tongue tissue showed weak punctate staining for EDB-FN primarily in the stroma, untreated primary OSCC and metastatic specimens demonstrated strong staining of malignant epithelial cells and surrounding stroma (FIG. 13a-c). These results are consistent to previous reported results of EDB-FN expression in human OSCC. Interestingly, tumors excised after neoadjuvant treatment exhibited reduced staining than untreated tumors, potentially suggesting EDB-FN downregulation after the treatment (FIG. 13d). These results underscore the potential for exploring EDB-FN as an oncoprotein target for molecular imaging of both primary and metastatic OSCC.

### Characterization of OSCC Cell Lines

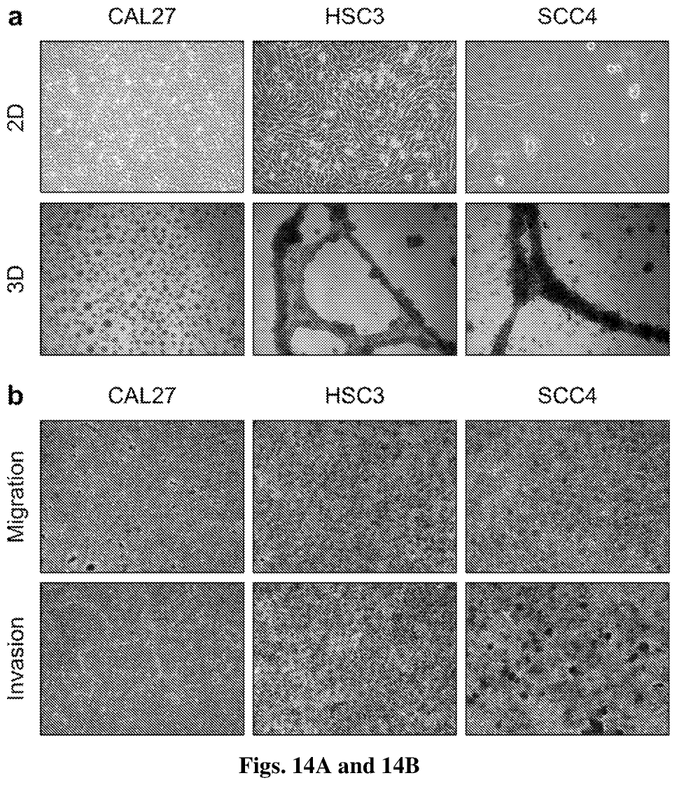

Three human OSCC cell lines were used to assess the effectiveness of MRMI of EDB-FN for diagnostic imaging of OSCC. CAL27 and SCC4 were isolated from the primary sites in the tongue before and after treatment, respectively. CAL27 maintains an epithelial, cobblestone-like layout of cells, while SCC4 is replete with polyploid giant cells (FIG. 14a). Interestingly, polyploid giant cells are often indicative of morphological changes due to treatment stress and have been associated with tumor recurrence, drug resistance, and heterogeneity. HSC3, on the other hand, was isolated from a cervical lymph node metastasis and has high metastatic potential among the tumor models.

When cultured on basement membrane extract for spheroid formation, CAL27 forms many small punctate spheroids while HSC3 and SCC4 form large dense networks that spread throughout the gel (FIG. 14a). Further, transwell assays were performed with and without Matrigel coating to characterize the invasive and migratory capacity of the cell lines, respectively. In both experiments, CAL27 showed limited ability to migrate or invade, while HSC3 and SCC4 showed substantial invasive potential (FIG. 14b). These data demonstrate that metastatic HSC3 and post-treatment SCC4 represent aggressive, high-risk OSCC, while CAL27 represents a less aggressive, low-risk OSCC.

EDB-FN is a Targetable Oncoprotein Associated with High-Risk OSCC

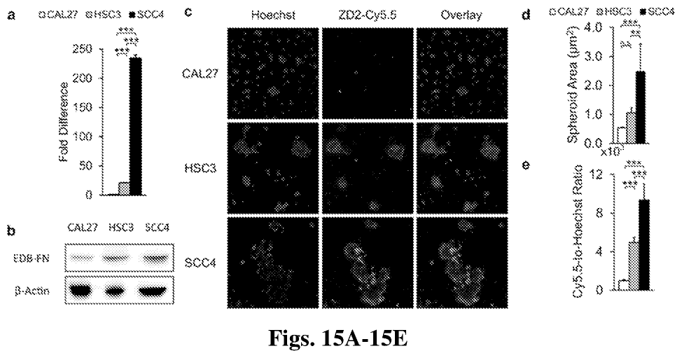

EDB-FN expression was analyzed to determine its correlation with aggressiveness in OSCC. qRT-PCR analysis showed significantly upregulated EDB-FN mRNA in invasive HSC3 (21-fold) and SCC4 (234-fold) cells relative to non-invasive CAL27 (FIG. 15a). Further, SCC4 exhibited 11-fold higher upregulated EDB-FN mRNA than HSC3. This result was corroborated on the protein level by western blotting, demonstrating that EDB-FN is differentially expressed and upregulated in invasive OSCC (FIG. 15b).

Spheroids were stained with an EDB-FN-specific fluorescence probe ZD2-Cy5.5 to assess targeting EDB-FN for molecular imaging of OSCC. Aggressive HSC3 and SCC4 cells formed larger spheroids with notably stronger staining of ZD2-Cy5.5 compared to CAL27 (FIG. 15c-d). Quantification of the staining intensity also revealed approximately 2-fold stronger staining of SCC4 than HSC3, in agreement with the EDB-FN expression analysis (FIG. 15e). This data demonstrates that the ZD2 peptide can effectively target differentially expressed EDB-FN for molecular imaging of OSCC.

### MT218 Differentially Enhances EDB-FN-Expressing OSCC in MRMI

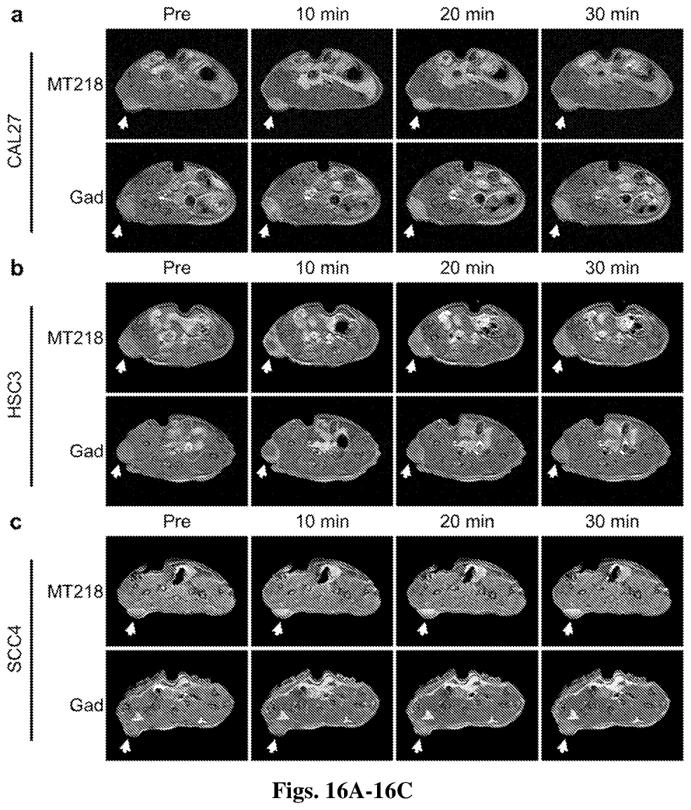

The effectiveness of MT218 for MRMI of EDB-FN in OSCC at a subclinical dose in correlation with tumor aggressiveness was determined in mouse flank xenograft models, using the clinical agent gadoteridol as an untargeted control. Low-risk, low-EDB-FN CAL27 tumors exhibited moderate enhancement with MT218 at 0.04 mmol/kg that washed out by 30 minutes post-injection. Enhancement with gadoteridol at 0.1 mmol/kg in CAL27 tumors appeared slightly greater than MT218 because of its high dose (FIG. 16a). High-risk, moderate-EDB-FN HSC3 tumors showed strong enhancement with MT218 at 10 minutes post-injection, which reduced significantly by 30 minutes. Enhancement with gadoteridol in HSC3 tumors appeared slightly less than, but generally comparable to, MT218 despite the dose reduction of the latter (FIG. 16b). High-risk, high-EDB-FN SCC4 tumors exhibited substantial enhancement with MT218 at all the time-points. Enhancement with gadoteridol in SCC4 tumors was much weaker than MT218 (FIG. 16c). These images suggest that MT218 differentially enhances high-risk OSCC tumors at a subclinical dose in accordance with their EDB-FN expression, a property unachievable with the clinical contrast agent.

### Semi-Quantitative Analysis of Contrast Enhancement

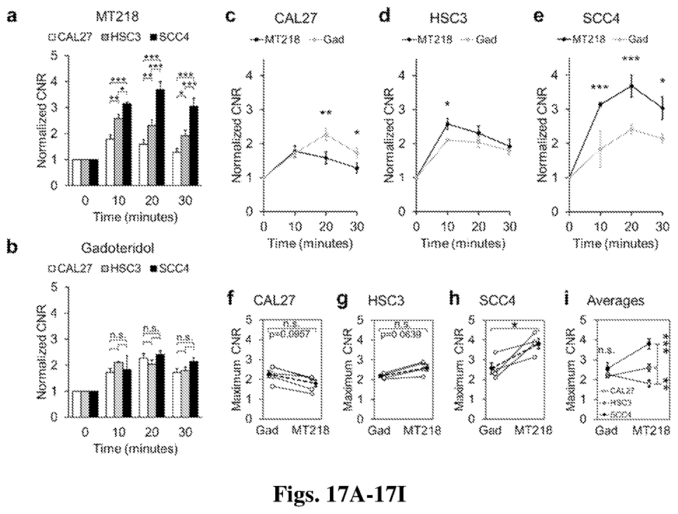

Contrast-to-noise ratios (CNR) in the tumors were calculated for semi-quantitative analysis of tumor enhancement with MT218 and gadoteridol. MT218 differentially enhanced the three tumor models at all the time-points in accordance with their EDB-FN expression, while gadoteridol showed no differential enhancement at any time point (FIG. 17a-b). Low-risk, low-EDB-FN CAL27 tumors showed significantly lower enhancement with MT218 than gadoteridol except at 10 minutes post-injection (FIG. 17c). High-risk, moderate-EDB-FN HSC3 tumors exhibited significantly higher enhancement with MT218 than gadoteridol at 10 minutes post-injection and trended higher for the remaining time-points (FIG. 17d). High-risk, high-EDB-FN SCC4 tumors exhibited significantly higher CNR enhancement with MT218 than gadoteridol at all the time-points (FIG. 17e). The average maximum enhancement between MT218 and gadoteridol was borderline significantly lower for CAL27 (CNRMT218=1.80±0.17, CNRgad=2.27±0.18, p=0.0957), borderline significantly higher for HSC3 (CNRMT218=2.61±0.17, CNRgad=2.18±0.07, p=0.0639), and significantly higher for SCC4 (CNRMT218=3.81±0.25, CNRgad=2.57±0.28, p=0.0170) (FIG. 17f-h). The average maximum enhancement with MT218 was significantly different between the three tumor models in accordance with their EDB-FN expression, while there were no significant differences with gadoteridol (FIG. 17i). These data validate the unique ability of MT218 to differentially enhance EDB-FN-expressing high-risk OSCC tumors in MRMI at a subclinical dose.

### Histological Analysis of Xenograft Tumors

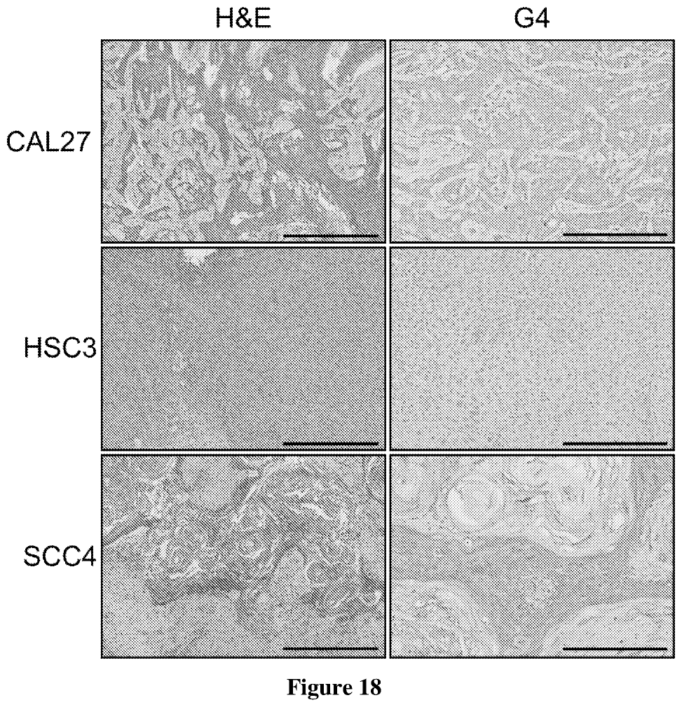

The tumors were excised post-imaging for histological and immunohistochemical analysis. Low-risk, moderately-differentiated CAL27 tumors exhibited weak EDB-FN staining in epithelial regions, with stromal staining increasing near the tumor boundary (FIG. 18). High-risk, poorly-differentiated HSC3 tumors exhibited stronger EDB-FN staining than corresponding regions in CAL27 tumors, with little stroma for interpretation (FIG. 18). High-risk, well-differentiated SCC4 tumors showed membranous staining for EDB-FN within and around epithelial regions that was the strongest of the three tumor models, while the large keratinizing stromal regions showed little staining (FIG. 18). The EDB-FN staining intensity of the epithelial tumor regions was in agreement with the in vitro EDB-FN expression analysis and the CNR analysis of MRMI data, suggesting that contrast enhancement in MRMI with MT218 of OSCC increases with increasing EDB-FN expression and can be used for differentiating aggressive from non-aggressive OSCC.

### Discussion

MRI is commonly used for the diagnosis of OSCC, providing unrivaled soft tissue contrast and high resolution that allows delineation of primary and metastatic disease and assists treatment planning. Additionally, MRI does not expose patients to harmful radiation, and GBCAs are typically less nephrotoxic at clinical doses than iodine-based CT contrast agents. In this study, MRMI with the targeted contrast agent MT218 provided robust enhancement in high-risk OSCC tumors at just 40% of the clinical dose. Similar results were observed in aggressive triple-negative breast cancer at just 20% of the clinical dose. Ayat et al. (2019), Front Oncol 9:1351. The increased enhancement is primarily derived from the selective binding of MT218 to EDB-FN in the tumor ECM, allowing accumulation and retention of MT218 in the tumor. Effective MRMI of aggressive tumors with MT218 at subclinical doses would significantly improve the clinical safety of GBCAs, mitigating potential dose-dependent side effects associated with gadolinium administration.

Lymph node metastasis is one of the most important prognostic indicators for OSCC, and occult metastasis occurs in 20-40% of patients with a clinically and radiologically negative neck. Standard practice to identify regional metastases includes physical examination and diagnostic imaging, but many metastases are regularly overlooked or unidentifiable due to their very small size. While elective neck dissection removes potentially affected lymph nodes, subsequent histological examination reveals many unnecessary dissections. We previously demonstrated that highly specific targeted GBCAs enable detection of micrometastatic breast cancer. Zhou et al., (2015), Nat Commun 6:7984. We also observed strong EDB-FN expression in metastatic OSCC specimens and cells, in agreement with previous studies. MRMI with the EDB-FN-targeting contrast agent MT218 therefore presents a promising new strategy for more accurate detection and diagnosis of OSCC metastases to improve precision management and personalized treatment of the disease.

Anti-cancer treatments can directly affect ECM protein expression, yielding a dynamic ECM that mediates tumor initiation, progression, and therapeutic efficacy. Harisi R, Jeney A (2015), Onco Targets Ther 8:1387-1398. Chemoresistance is regularly associated with increased AKT signaling and downstream fibronectin and EDB-FN expression. We observed reduced EDB-FN expression in neoadjuvant OSCC specimens and increased expression in OSCC cells after long-term exposure to treatment. In addition, more than 20% of head and neck squamous cell carcinoma patients experience locoregional tumor recurrence, after which the 5-year survival rate falls to 30%. MRMI with MT218 has the potential to associate and visualize treatment response and changes in EDB-FN expression, providing physicians insight into the acquisition of high-risk features like drug resistance to improve timely and personalized treatment, as well as monitor for tumor recurrence post-resection for precision management of the disease.

This preclinical study has several limitations. The role of EDB-FN in cancer biology is largely unclear and needs further investigation at the molecular, preclinical and clinical levels. Further investigations are also needed for comprehensive evaluation of EDB-FN as a viable biomarker for aggressive OSCC. While the MRI technique used in this study is semiquantitative, the use of MT218 in quantitative MRI techniques, e.g. MR fingerprinting, could provide more accurate measurement of EDB-FN expression in OSCC in correlation with tumor invasiveness and therapeutic response. Future work will focus on the validation of EDB-FN as an imagable biomarker of OSCC and clinical translation of MRMI with MT218 for precision imaging of OSCC.

### Conclusions

We showed the preliminary correlation between EDB-FN expression and aggressiveness of human OSCC cells, tumors models, and patient specimens. MRMI of EDB-FN with MT218 provided substantial and differential contrast enhancement in OSCC tumor models in correlation with their inherent EDB-FN expression and aggressiveness at a subclinical dose. Gadoteridol meanwhile provided modest undifferentiated enhancement at the clinical dose. The results indicate that MRMI with the EDB-FN-targeting contrast agent MT218 has the potential to provide accurate diagnosis, risk assessment, delineation, and therapeutic efficacy monitoring in clinical management of OSCC.

### Example 4: Overcoming Stromal Barriers by Targeting an ECM Oncoprotein for Early Detection of Pancreatic Cancer with MR Molecular Imaging

Pancreatic cancer (PaCa) is responsible for a large and rapidly growing number of cancer deaths. PaCa prognosis remains poor, with a five-year survival rate of merely 9%. Patients often present with advanced-stage PaCa that has metastasized or cannot be surgically resected. Analysis of post-surgical outcomes suggests that the detection and removal of early-stage disease results in dramatically improved survival or disease cure. However, current strategies for PaCa diagnosis are not sensitive for early-stage disease. Contrast enhanced computed tomography (CE-CT) is the most commonly utilized for imaging of PaCa, but has difficulty for diagnosing small and potentially curable tumors, lymph node metastasis, and liver metastasis. Contrast enhanced magnetic resonance imaging presents superior soft tissue contrast and excellent spatial resolution, and is increasingly utilized in PaCa diagnosis. However, the existing clinical contrast agents are not tumor-specific, and suffer from poor sensitivity in detecting small tumors. Development of tumor-specific contrast agents would improve intratumoral contrast agent accumulation and maximize the advantages of MRI for accurate detection and delineation of early-stage PaCa. There is an unmet need for safe and effective targeted MRI contrast agents that can detect early-stage PaCa.

Pancreatic cancer has a dense tumor stroma that impedes binding of molecular imaging agents that target cell-surface molecules and presents a formidable barrier for effective molecular imaging and cancer detection. Nevertheless, its unique extracellular matrix (ECM) molecular signature can be exploited to generate image contrast for precision molecular imaging and detection of small tumors. Extradomain B fibronectin (EDB-FN) is an oncofetal splice variant of fibronectin, and is reestablished in malignancy, but absent in most normal tissues. Han et al. Bioconjugate Chemistry 26, 830-838 (2015). EDB-FN in the tumor ECM is readily accessible for specific binding of an imaging agent for effective molecular imaging of PaCa tumors. Its abundance in aggressive tumors allows rapid binding of sufficient targeted contrast agent to generate robust signal enhancement in magnetic resonance molecular imaging (MRMI). Therefore, EDB-FN is a promising target molecular imaging and early detection of PaCa with MRMI.

We previously developed an oligopeptide, TVRTSAD (ZD2), that binds with high specificity to EDB-FN17. ZD2 targeted MRI contrast agents have been developed and tested for MRMI of EDB-FN in aggressive breast cancer and prostate cancer models. Han et al. Nature Communications 8, 692 (2017). The targeted contrast agent ZD2-N3-Gd(HP-DO3A) (MT218) was developed by conjugating ZD2 peptide to a clinical macrocyclic contrast agent Gd(HP-DO3A)17,21. MT218 has a higher T₁ relaxivity than Gd(HP-DO3A) and has demonstrated superior contrast enhancement in aggressive breast and prostate cancers. Ayat et al., ACS Medicinal Chemistry Letters, 9(7):730-735 (2018). The binding of MT218 to abundant EDB-FN in PaCa tumor ECM has the potential to overcome the barriers of the dense tumor stroma to generate strong contrast enhancement to improve PaCa imaging with MRMI.

### Materials and Methods

### Cells and Reagents

ZD2-N3-Gd(HP-DO3A) was obtained from Molecular Theranostics (Cleveland, OH). ProHance®, Gd(HP-DO3A), was purchased from Bracco Diagnostics (Monroe Township, NJ). ZD2-Cy5.5 was synthesized as previously described. Han et al., Nature Communications 8, 692 (2017). Capan-1, BxPC3, and PANC-1 (ATCC, Manassas, VA) human PaCa cells were cultured in recommended culture media and conditions. BxPC3 and PANC-1 cells were transduced with a lentiviral vector (Amsbio, Cambridge, MA) for expression of green fluorescent protein (GFP) and luciferase.

### Relaxivity Measurements

The T₁ and T₂ values of MT218 and Gd(HP-DO3A) solutions were determined using a 3T MRS 3000 scanner (MR Solutions, Guildford, UK). Solutions of MT218 were prepared at concentrations of 0, 0.25, 0.5, 1, and 2 mM in DPBS buffer (Gibco, Gaithersburg, MD). T₁ values were measured using an inversion recovery-fast low angle shot (IR-FLASH) sequence (i=10 ms, TE=4 ms, FA=8°, echoes/frames=128, FOV=40 mm×40 mm, slice thickness =2 mm, number of averages=1, time delay=4000 ms, sample period=200, matrix=256×128). T₂ values were obtained using a multi-echo multi-slice (MEMS) sequence (TE=15 ms, repetition time=15 ms, echoes=10, FOV: 40 mm×40 mm, slice thickness=1 mm, number of averages=1, matrix=256×192). The r₁ and r₂ relaxivities were calculated from the slopes of 1/T₁ and 1/T₂ vs. Gd concentration plots, respectively.

### Binding Affinity Measurements

The binding affinity of MT218 was determined with microscale thermophoresis (MST) using a Nanotemper™ Monolith NT.115 instrument (NanoTemper Technologies, Munich, Germany). The EDB fragment of FN was expressed in E. coli, purified, and labelled with amine-reactive dye NT-647. MT218 was dissolved into assay buffer (50 mM PBS w/0.05% Tween-20). This solution was further diluted using assay buffer to give a series of MT218 working solutions. Each MT218 working solution was then mixed with a fixed concentration of fluorescently labeled EDB fragment. The mixtures of MT218 and EDB were then loaded into standard capillaries, and MST measurements were performed at 25° C. using 20-80% light-emitting diode power and 40% infrared-laser power at varying concentrations of MT218.

### Animal Models

For flank xenograft models, 4×10⁶ Capan-1 cells suspended in 100 μL of a 6 mg/mL Matrigel (Corning, Corning, NY) solution was injected subcutaneously into the flank of eight-week-old female nu/nu mice. The tumor bearing mice were imaged after tumor volumes exceeded 300 mm³. For orthotopic intrapancreatic models, the pancreas of four-week-old female nu/nu mice were surgically exposed and 1×10⁶ BxPC3-GFP-Luc or PANC-1-GFP-Luc cells suspended in 100 μL of an Matrigel solution (6 mg/mL) was injected into the pancreatic tissue. Prior to imaging, growth of tumor was verified by bioluminescence imaging with intraperitoneal injection of d-luciferin on IVIS Spectrum system (Perkin Elmer, Waltham, MA).

### ZD2-Cy5.5 Staining of Human Tissue

Tissue sections from PaCa patients (Case Comprehensive Cancer Center, Cleveland, OH) were deparaffinized with xylene, ethanol, and washed with water. Blocking was performed with 10% goat serum (Invitrogen, Carlsbad, CA) in PBS with 0.1% Tween 20 (PBS-T) for 30 minutes and incubated with 500 nM ZD2-Cy5.5 in PBS-T for 1 hour at 37° C. Following three washes with PBS-T, the sections were mounted using Fluoroshield mounting medium (Abcam, Cambridge, UK). Images were acquired on a FV1000 (Olympus, Waltham, MA) confocal microscope using pre-programmed emission and excitation filters for Cy5.5 (excitation: 635 nm; emission: 693 nm) and DAPI (excitation: 405 nm; emission: 461 nm), using a 10× objective lens. All tissue samples were imaged at the same laser power and sensor gain. Images were generated from single-channel grayscale images using FIJI software.

### Western Blotting Analysis of Cells and Tissue

Capan-1, BxPC3, and PANC-1 cells and tissues were lysed in RIPA buffer supplemented with cOmplete™ protease inhibitor cocktail (Roche, Basel, Switzerland). Tissue samples were further homogenized with a rotor-stator homogenizer (IKA, Wilmington, NC). Lysates were centrifuged and the supernatants were assayed for total protein concentration using the BCA protein assay (Thermo Fisher Scientific, Waltham, MA). Total protein (30 μg) was mixed in Laemli buffer (Bio-Rad, Hercules, CA) and boiled for five minutes. Samples were separated by SDS-PAGE (5-20%) and transferred onto nitrocellulose membrane (Cell Signaling Technologies, Danvers, MA). Primary antibodies used were anti-EDB-FN antibody BC-1 (1:500; Abcam, Cambridge, MA) diluted in 5% bovine serum albumin and anti-$-Actin antibody (1:1000; Cell Signaling Technologies, Danvers, MA) diluted in 5% milk for cell lysates. G4 antibody (1:1000; Absolute Antibody, Boston, MA) in 5% bovine serum albumin and anti-GAPDH antibody (1:1000; Cell Signaling Technologies, Danvers, MA) in 5% milk were used for tissue lysates. Primary antibody incubation was performed overnight. Secondary anti-mouse-HRP (1:1000; Cell Signaling Technologies, Danvers, MA) and anti-rabbit-HRP (1:1000; Cell Signaling Technologies, Danvers, MA) antibodies were incubated at room temperature in 5% milk in TBS-T for 1 hour with shaking. The stained membranes were developed with a SignalFire Plus ECL kit (Cell Signaling Technologies, Danvers, MA) and imaged using the ChemiDoc XRS+ system (Bio-Rad, Hercules, CA). Densiometric measurements were made with FIJI software.

### Immunohistochemical and Histochemical Staining of Mouse Tumor Tissue

PaCa tumor tissue and normal pancreatic tissue were collected from euthanized mice and fixed with 10% buffered formalin for 24 hours. The samples were embedded in paraffin blocks and 5 μm sections were cut using a RM2235 microtome (Leica, Buffalo Grove, IL). Hematoxylin/eosin staining was performed under standard conditions. Antigen retrieval was performed at 125° C. for 30s in pH 6.0 citrate buffer, followed by 3% H₂O₂ peroxidase block (8 min) and Rodent Block M (20 min) (Biocare Medical, Pacheco, CA). Anti-EDB-FN G4 monoclonal antibody (1:100) was incubated with tissue sections at RT for 1 hour with shaking. Detection was performed with HRP Polymer detection solution (Biocare Medical, Pacheco, CA). Visualization was performed with 3,3′-diaminobenzidine for 5 min and counterstained with hematoxylin for 5 s. Images were acquired with an Bx61VS (Olympus, Waltham, MA) slide scanner and processed in OlyVIA software. Histological interpretation was performed by a board-certified pathologist.

For assessment of binding specificity of ZD2 peptide, flash frozen samples of tumor and normal tissues were collected from the euthanized tumor bearing mice and embedded in optimal cutting temperature media. The embedded samples were then immersed in liquid nitrogen for 5 minutes and transferred to −80° C. for storage before cryosectioning. Tissue sections were blocked for 1 hr in PBS containing 0.05% Tween-20 and 10% normal goat serum (Gibco, Gaithersburg, MD) and then stained with BC-1 primary monoclonal antibody (1:400) or ZD2-Cy5.5 (500 nM) at 4° C. overnight or room temperature incubation for 2.5 hr, respectively, and then washed with PBS-T buffer. Secondary Alexa Fluor 594 anti-rabbit was incubated with the BC-1 treated sections for 1 hour at room temperature (1:1000). For the blocking experiment, the tissue sections were first incubated with an excess of BC1 antibody at 4° C. overnight, then washed three times with PBS-T. The sections were then incubated with ZD2-Cy5.5 at room temperature incubation for 2.5 hr. The stained sections were washed with PBS-T buffer and images were acquired on an Olympus FV1000 (Waltham, MA) confocal microscope using pre-programmed emission and excitation filters for Cy5.5 (excitation: 635 nm; emission: 693 nm) and DAPI (excitation: 405 nm; emission: 461 nm), using a 10× objective lens.

### Contrast-Enhanced MRI

Flank tumor-bearing mice were anesthetized and a tail vein catheter inserted. MR image acquisition was performed on a 7T Biospin (Bruker, Billerica, MA) preclinical small animal scanner with a volume radiofrequency coil. Axial images were obtained using a multi-spin multi-echo (MSME) MRI sequence (500 ms TR, 8.1 ms TE, 900 FA, 4.50 cm×4.50 cm FOV, 1.50 mm slice thickness, 16 slices, 2 averages, 200×200 matrix, 0.0225×0.0225 cm/pixel resolution). Contrast agents were injected at a dose of 0.1 mmol/kg (100 μL) via the tail vein catheter, followed by a saline flush. Images of the tumors were acquired before contrast and at various time points after contrast administration. Gd(HP-DO3A) was used as a control.

Mice bearing intrapancreatic tumors were anesthetized and a tail vein catheter was inserted. Image acquisition was performed on a MR Solutions 3T MRS 3000 scanner with a mouse radiofrequency coil. Axial images were obtained using a Fast Spin Echo (FSE) MRI sequence (305 ms TR, 11 ms TE, 90° F.A, 4 cm×4 cm FOV, 1 mm slice thickness, 10 slices, echo train length=4, echo spacing=11 ms, 4 averages, 256×256 matrix, 0.0156×0.156 cm/pixel resolution) with respiratory gating. Images were acquired before contrast and at different time points after contrast injection at a dose of 0.1 mmol/kg (100 μL). For competitive binding experiments, a 5:1 molar ratio of free ZD2 peptide to MT218 was injected in mice bearing intrapancreatic BxPC3 tumor xenografts (n=3) at a dose of 0.1 mmol MT218/kg (100 μL). Images obtained on the MR Solutions 3T scanner were normalized using the Surscalereader.exe (MR Solutions, Guildford, UK) program supplied.

Image files were exported and analyzed in Horus software. ROIs were drawn in the tumor, liver, kidney, and muscle. Muscle ROIs were taken from muscle, with other structures (liver, kidney, tumor) identified after comparison with published images and comparison with anatomical landmarks or bioluminescent imaging. Size of ROIs drawn for image analysis are summarized in Supplemental Table S1. The contrast-to-noise ratio of the tumor (CNR) was calculated using the following equation, where σ_noise is the standard deviation of intensities from an ROI drawn outside of the mouse body:

$$CNR = \frac{Mean {Intensity}_{Tumor} - Mean {Intensity}_{Muscle}}{{σ}_{noise}} .$$

The contrast to noise ratio of the liver (CNR) was calculated using the following equation:

$$CNR = \frac{Mean {Intensity}_{Liver} - Mean {Intensity}_{Muscle}}{{σ}_{noise}}$$

Image subtraction was performed on images adjusted to the same window and level in FIJI open-source software. Result of image subtraction was displayed using the RGB Rainbow color lookup table.

### Statistical Analysis

Image contrast was reported as fold enhancement of contrast to noise ratio of postcontrast images over precontrast images using Fold Enhancement=CNRₚₒₛₜ/CNRₚᵣₑ. Statistical analysis of CNR was performed in Minitab Express (Minitab Inc, State College, PA). One-way ANOVA with Fisher Individual Tests for Differences of Means post-hoc testing was performed to determine statistical significance between greater than two means (p<0.05). Student's T test was used to determine statistical significance between two means (p<0.05).

### Results

### Binding Affinity and Relaxivities of MT218

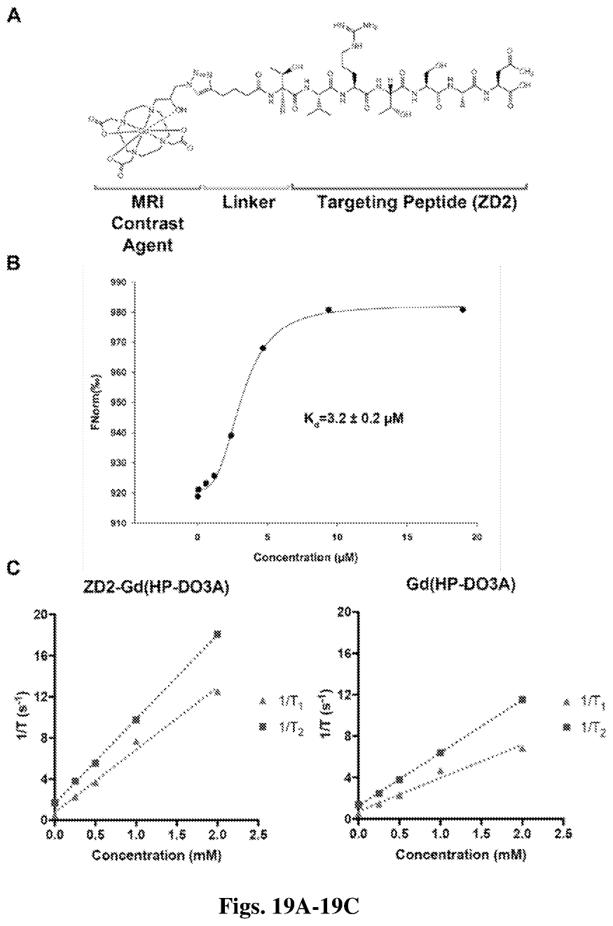

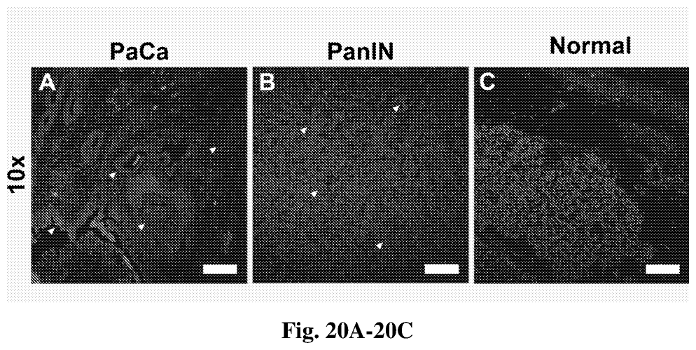

FIG. 19 shows the chemical structure, binding affinity, and relaxivities of the targeted contrast agent MT218. MT218 is a small molecular conjugate of ZD2 peptide to a macrocyclic clinical MRI contrast agent Gd(HP-DO3A). The binding affinity of MT218 to EDB-FN was measured to be 3.2±0.2 μM. The T₁ and T₂ relaxivities of MT218 were measured to be 6.07±1.13 s⁻¹ mM⁻¹ and 8.20±0.18 s⁻¹ mM⁻¹, respectively, at 3 T. T₁ and T₂ relaxivities of Gd(HP-DO3A) were measured to be 3.20±0.96 s⁻¹ mM⁻¹ and 5.12±0.18 s⁻¹ mM⁻¹, respectively, at 3 T.

### EDB-FN Expression in Human PaCa

FIG. 20 shows the binding of ZD2 peptide to EDB-FN in human PaCa, pancreatic intraepithelial neoplasia (PanIN), and normal pancreatic tissue specimens with the targeted fluorescent probe ZD2-Cy5.5. ZD2-Cy5.5 binding was high in human PaCa tissue, moderate in precancerous PanIN tissue, and low in normal pancreatic tissue. The distribution of ZD2-Cy5.5 binding was heterogenous in malignant and premalignant tissues. Strong fluorescence intensity was seen in poorly organized ductal structures in the PaCa specimen, while intermediate staining was observed in the PanIN tissue with staining concentrated in the cell clusters with no luminal structure. The fluorescence intensity of ZD2-Cy5.5 binding in different human pancreatic tissues was indicative of high expression of EDB-FN in malignant PaCa tissue, intermediate expression in precancerous PanIN, and low expression in normal pancreas.

### EDB-FN Expression in Murine Models of PaCa

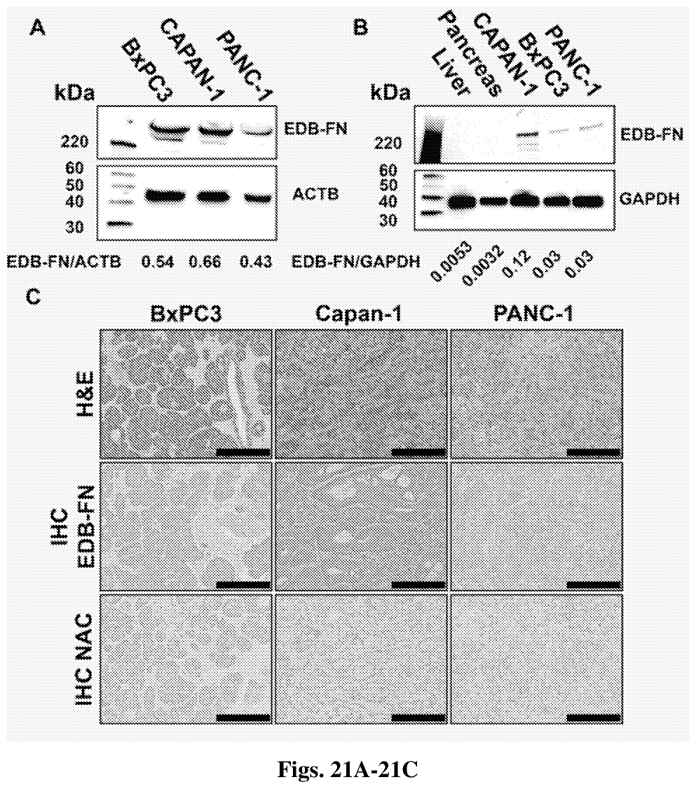

The expression of EDB-FN was also evaluated in Capan-1, BxPC3-GFP-Luc, and PANC-1-GFP-Luc human PaCa cells and tumor xenografts derived from the cells. Western blotting with the EDB-FN specific G4 antibody revealed the expression of two 220+ kDa bands consistent with the size of EDB-FN protein in all three PaCa lines (FIG. 21A). Western blotting of liver, pancreas, and tumor protein extracts from mice bearing the PaCa xenografts also showed the 220+ kDa band in all three PaCa models and not in the normal hepatic and pancreatic tissue, indicating elevated EDB-FN expression in the tumors and no expression in the normal tissues and organs (FIG. 21B). Hematoxylin and eosin stained slides of the pancreatic tumor xenografts revealed large nodules of poorly differentiated carcinoma consistent with the appearance of PaCa (FIG. 21C). All tumors exhibited densely packed cells with low cytoplasmic to nuclear ratio. Immunohistochemical staining with G4 anti-EDB-FN antibody exhibited strong staining throughout the tissue sections of all three PaCa models, while no staining was observed without the antibody (FIG. 21C). Staining of flash frozen tissue sections with an EDB-FN specific monoclonal antibody BC-1 or ZD2-Cy5.5 revealed similar staining patterns for EDB-FN in BxPC3, Capan-1, and PANC-1 tissues. No staining was seen in normal pancreatic and muscle tissues, indicating no EDB-FN expression. ZD2-Cy5.5 binding was blocked in the presence of the anti-EDB-FN antibody. The results indicate that EDB-FN is overexpressed in human pancreatic cancer cells and their tumor xenografts in mice with no expression in normal tissues.

### MRMI of Pancreatic Cancer Flank Xenografts

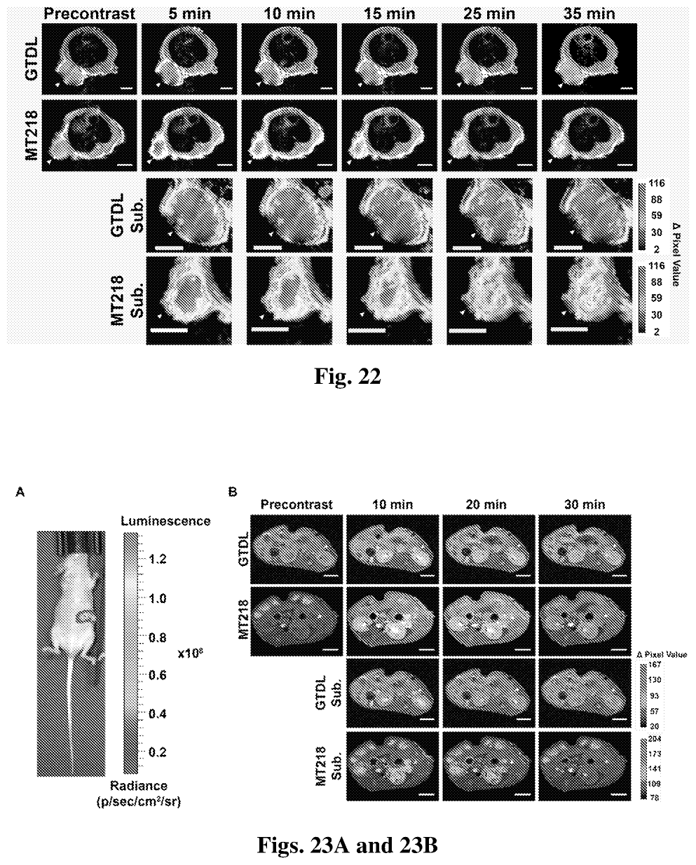

The effectiveness of MRMI of EDB-FN with the targeted contrast agent MT218 in detecting PaCa was first assessed in mice bearing subcutaneous Capan-1 flank tumor xenografts. The clinical agent Gd(HP-DO3A) was used as a control at the same dose (0.1 mmol/kg). Stronger contrast enhancement was observed in the periphery of the tumors 5 minutes after injection with MT218 than with Gd(HP-DO3A) (FIG. 22). The contrast enhancement in the tumor core then gradually increased in the mice injected with MT218 (FIG. 22). The mice administered with Gd(HP-DO3A) did not exhibit significant tumor core enhancement. Subtraction of the precontrast images from the postcontrast images at different time points of the same mice illustrated the strong initial enhancement in the tumor periphery at the early time points (5-15 min postcontrast) and then robust tumor contrast enhancement throughout the tumors at later time points (25-35 min postcontrast) after MT218 administration (FIG. 22). In comparison, the subtraction images of the mice administered Gd(HP-DO3A) produced modest peripheral contrast enhancement at early time points postinjection, and little to no intratumoral contrast at all time points. The results demonstrate that MT218 is effective in producing robust signal enhancement throughout the tumors for effective molecular imaging and detection of PaCa with MRMI.

### MRMI of Intrapancreatic Tumor Xenografts

The effectiveness of MRMI with MT218 was further investigated in mice bearing intrapancreatic human PaCa tumor xenografts. Bioluminescence imaging verified the presence of luciferase-labeled BxPC3-GFP-Luc tumor xenografts in the left upper abdomen of three mice implanted with tumor cells (FIG. 23A). MT218 MRMI of tumor bearing mice clearly delineated small lesions with strong contrast enhancement within 10 minutes of MT218 administration (FIG. 23B). Tumor contrast enhancement then decreased at 20 min and returned to background levels 30 min after MT218 injection. The clinical agent Gd(HP-DO3A) generated modest contrast enhancement in the tumors at 10 min post-injection and the signal enhancement reduced to background level 20 min after Gd(HP-DO3A) injection (FIG. 23B). Subtraction of the precontrast images from the postcontrast images further demonstrated strong enhancement and clear delineation of the intrapancreatic tumors with the targeted contrast agent at 10 min postinjection in T₁-weighted MR images, while Gd(HP-DO3A) produced less intratumoral signal enhancement (FIG. 23B). To further demonstrate the specificity of MT218 for MRMI of PaCa, a competitive binding experiment was performed with co-administration of a 5-fold excess of free ZD2 peptide and MT218 in mice bearing intrapancreatic BxPC3 xenografts. The co-injection of the excess free peptide blocked the binding of the MT218 to the tumors, resulting in significant reduction of contrast enhancement in the tumor images as compared to the tumor-bearing mice injected with MT218 with free ZD2 peptide.

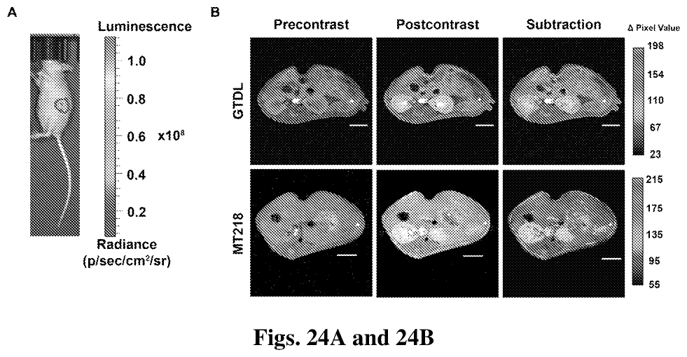

To determine whether the improved contrast enhancement in MRMI with MT218 is reproducible, MT218 MRMI was tested in another intrapancreatic PaCa model derived from PANC-1-GFP-Luc cells. Bioluminescence images of the mice bearing PANC-1-GFP-Luc intrapancreatic xenografts revealed substantial luciferase signal in the upper left abdominal region of cancer cell inoculation, indicating the tumor presence (FIG. 24A). MT218 MRMI of tumor-bearing mice generated substantial contrast enhancement in tumor tissues and clearly delineated the tumors at 10 minutes post-injection. In contrast, Gd(HP-DO3A) did not produce significant tumor contrast enhancement and was not able to clearly identify the tumors (FIG. 24B). Subtraction images of the precontrast images from the postcontrast images reveal substantial signal enhancement in the tumor with MT218, whereas little change was observed after Gd(HP-DO3A) administration (FIG. 24B).

### Quantitative Analysis of ZD2 MRMI

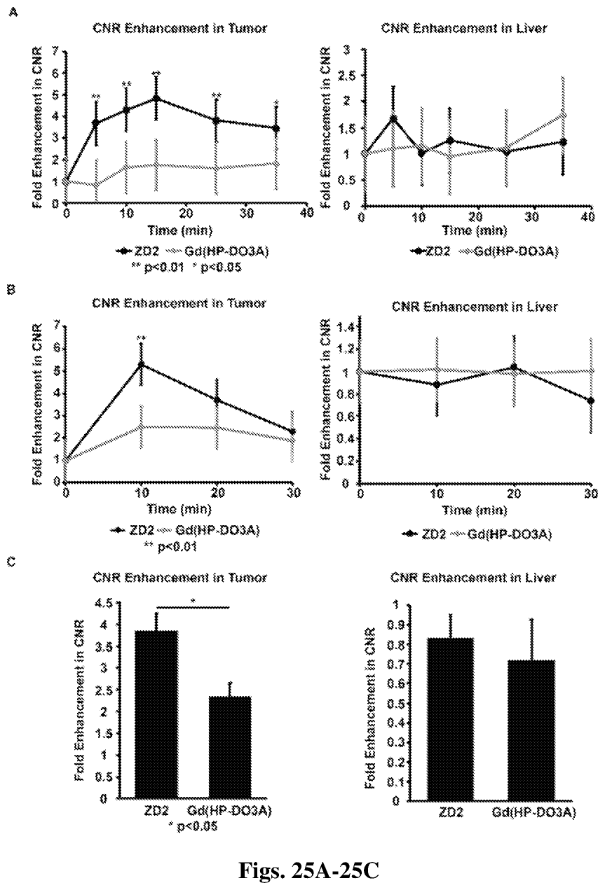

The MRMI tumor signal enhancement of MT218 was analyzed in comparison with the clinical agent. The signal enhancement in the liver was also analyzed to determine the potential nonspecific contrast enhancement of MT218 in normal tissues. MT218 generated a 4.84-fold increase of contrast to noise ratio (CNR) in the Capan-1 flank tumor xenografts at 15 minutes post-injection that maintained for at least 35 minutes (FIG. 25A). The clinical agent Gd(HP-DO3A) produced an intratumoral CNR increase of 1.77 fold for the duration of the experiment, which was significantly less than MT218 (p<0.01). No significant difference in CNR was observed in the liver (FIG. 25A), kidney, and spleen of all tested mice between the two contrast agents (p>0.05).

MT218 produced a maximum 5.3-fold CNR increase in the BxPC3-GFP-Luc intrapancreatic tumors at 10 min post-injection, while Gd(HP-DO3A) produced approximately 2.5-fold tumor CNR increase at 10 min, significantly less than that with MT218 (p<0.01) (FIG. 24B). The tumor CNR subsequently decayed for both agents. Similarly, MT218 produced approximately 3.85-fold intratumoral CNR increase in the PANC-1-GFP-Luc intrapancreatic tumor xenografts 10 min postinjection, whereas Gd(HP-DO3A) produced a 2.35-fold tumor CNR increase (FIG. 25C). No difference in CNR was observed in the liver (FIG. 25B,C) and kidneys of all tested mice between the two contrast agents (p >0.05).

Co-injection of an excess of free ZD2 peptide with MT218 resulted in approximately 2.5 fold CNR increase in the BxPC3-GFP-Luc intrapancreatic model, which was similar to the CNR increase with Gd(HP-DO3A) (FIG. 25B). The CNR increase with MT218 in the presence of 5-fold free peptide was significantly lower than the CNR increase with MT218 without the free peptide (ca. 4.5 fold, p<0.05). The co-injection of free ZD2 peptide inhibited the binding of MT218 and reduced tumor CNR to the level of non-specific accumulation of the agent similar to that of Gd(HP-DO3A). The results indicate the specific binding of MT218 to PaCa tumors.

### Discussion

The clinical use of magnetic resonance molecular imaging (MRMI) is desirable for precision cancer imaging as it combines the high soft tissue contrast and good spatial resolution of MRI with the ability to image and characterize molecular changes within tumors. A major challenge to clinical MRMI is to overcome the stromal barrier for sufficient binding of a targeted contrast agent to generate detectable contrast enhancement. Pancreatic cancer (PaCa) is highly fibrotic with a dense ECM, which limits the access of contrast agents to the inner tumor tissues. Many molecular imaging agents are bulky and bind to cell-surface targets that are difficult to reach. By exploiting oncospecific expression of extradomain B fibronectin (EDB-FN) in the tumor ECM as a target for MRMI, we found that the imaging of PaCa could be substantially improved.

We found that expression of EDB-FN could be detected in human PaCa and precancerous tissues with the EDB-FN specific fluorescent probe ZD2-Cy5.5, suggesting that MT218 MRMI may also be useful for characterizing premalignancy and malignancy. The expression of EDB-FN in PaCa and premalignancy is consistent with the observations of other groups. Jailkhani et al. Proceedings of the National Academy of Sciences, 116, 14181 (2019). The effectiveness of EDB-FN MRMI for detection of PaCa was demonstrated in mouse models of PaCa using the molecular imaging agent ZD2-N3-Gd(HP-DO3A) (MT218). MT218 binds to EDB-FN with micromolar affinity, consistent with previous reports. Han et al., Bioconjugate Chemistry 28, 1031-1040, (2017). Furthermore, the relaxivity of MT218 is higher than that of Gd(HP-DO3A). MT218 generates substantially greater image contrast-to-noise (CNR) compared to the clinical agent Gd(HP-DO3A) in Capan-1 flank (273% CNR of control, p<0.05), BxPC3-GFP-Luc (212% CNR of control, p<0.05) and PANC-1-GFP-Luc intrapancreatic (164% CNR of control, p<0.05) murine models of PaCa due to its specific tumor binding and high T₁ relaxivity. The specific binding of MT218 to the EDB-FN enriched PaCa was validated by the decreased tumor CNR due to the disruption of MT218 binding by an excess of free ZD2 peptide in the intrapancreatic model. Time course MRMI images reveal that MT218 is able to bind to EDB-FN deep within the tumor core, which was not observed in imaging studies utilizing the untargeted clinical contrast agent Gd(HP-DO3A). Moreover, the small size (1442 Da), moderate binding affinity, and hydrophilicity of MT218 contribute to its mobility for diffusion and binding within the tumor core while maintaining low background noise. Tumor nodules with area as small as 6.13 mm² were better visualized with MT218, suggesting that MT218 has the potential for accurate early detection of PaCa that may improve early detection to initiate timely treatment for better patient survival. This is consistent with the performance of other ZD2 targeted imaging agent, which was able to detect small metastases using MRMI28. This is critical for timely initiation of therapy and improved therapeutic outcomes. It has been reported that detection and surgical removal of PaCa tumors less than 10 mm in size will greatly improve the long-term survival and quality of life of PaCa patients. Tsunoda et al., Journal of Hepato-Biliary-Pancreatic Surgery 5, 128-132, (1998).

Molecular imaging of EDB-FN in cancer has been previously investigated with antibodies labeled with radioisotopes for PET imaging. Anti-EDN-FN antibody BC-1 was labeled with ¹²⁵I and tested in mice bearing U87 brain tumor and SKMel28 melanoma xenografts. Mariani et al., Cancer 80, 2378-2384 (1997) Although the antibody is highly specific to EDB-FN and was found to bind to the EDB-FN rich tumors, its long-term retention in the circulation due to its large size generated significant background noise and affected the quality of specific tumor imaging. Nanobodies with a smaller size were recently developed for molecular imaging of EDB-FN in various types of tumor models. A ⁶⁴Cu-labeled nanobody probe (⁶⁴Cu-NJB2) demonstrated specific uptake and effective detection of PaCa and premalignant lesions in a mouse model utilizing PET/CT26. ZD2 peptide has also been labeled with ⁶⁸Ga as a PET probe for molecular imaging of EDB-FN. The ZD2 targeted probe provided sensitive and specific molecular imaging of EDB-FN in PaCa. Gao et al., Am J Nucl Med Mol Imaging 9, 216-229 (2019). Compared to PET, MRMI is advantageous for the delineation of small PaCa with high resolution and soft tissue contrast, which is valuable for treatment planning. The clinical implementation of PET/MRI provides a unique approach for molecular imaging of PaCa by targeting EDB-FN with PET probes and MRI contrast agents.

Other approaches to EDB-FN MRMI are also being investigated. Dextran-based chemical exchange saturation transfer (CEST) MRI has also utilized the ZD2 peptide to image PaCa. Han et al., Bioconjugate Chemistry 30, 1425-1433 (2019) The Dextran-ZD2 conjugate generated detectable intratumoral signal in a flank model of PaCa over 45 minutes, supporting the hypothesis that MRMI of EDB-FN provides diagnostic value. However, CEST faces several challenges to translation, including lower signal to noise ratio at clinical field strengths, low sensitivity, and high doses. So far, gadolinium based contrast agents, especially the macrocyclic agents, are considered as the safe and effective contrast agents for clinical cancer MRI.

Although we have shown the promise of MRMI of EDB-FN in PaCa imaging, there are limitations to this study. Comprehensive investigation of EDB-FN expression in a large human PaCa population is needed to validate it as a biomarker for PaCa. It is possible that EDB-FN expression may vary greatly in human PaCa. However, the high degree of correlation between our results and another small-scale study of EDB-FN expression in human PaCa suggests that this is unlikely to be the case. The models utilized for this study may not fully recapitulate the tumor microenvironment of human PaCa due to the impairment of immune function in nu/nu mice. Further studies of MT218 are currently being conducted in immunocomcompetent and spontaneous models of PaCa. There is also a need for further epidemiological investigation of relevant risk factors and screening tools that would work in concert with MRMI to detect early pancreatic cancer. MRMI can improve the detection of early stage PaCa, especially for high risk populations, but is not an ideal screening tool in the general population. It is our belief that clinical translation of MRMI with ZD2-N3-Gd(HP-DO3A) will facilitate the rapid development of general population screening tools that can identify high risk patients who may benefit from MRMI detection of early stage PaCa.

In summary, this study investigates the overexpression of EDB-FN in human PanIN, PaCa specimens, and in murine models of PaCa, and demonstrates the effectiveness of MRMI of EDB-FN with a small molecular targeted MRI contrast agent MT218. MRMI with MT218 generates superior contrast enhancement and clearly delineates small PaCa tumors. Minimal non-specific signal enhancement was observed in the hepatic tissue. MRMI with MT218 has the potential for surveillance of precancerous pancreatic lesions and for precision detection and delineation of small pancreatic cancer. Clinical translation of MRMI with MT218 has the promise to addresses the unmet clinical need for a highly specific imaging technology to detect early-stage pancreatic cancer, and to impact a variety of aspects of clinical management of pancreatic cancer, including screening the high-risk populations, diagnosis, treatment decision making, and post-treatment surveillance and monitoring.

### Example 5: Contrast Enhanced MRMI of EDB-FN Using MT218 Facilitates Non-Invasive Monitoring and Active Surveillance of Prostate Cancer

The potential role of EDB-FN as a molecular marker for MRMI with MT218 was determined in multiple models of prostate cancer of varying degrees of aggressiveness.

The low-risk, low-EDB-FN-expressing prostate cancer LNCaP cell line was modified to generate more invasive prostate cancer cells. For example, LNCaP-CXCR2 cells were designed to stably overexpress the pro-inflammatory and pro-tumorigenic IL8 receptor CXCR2. C4-2 cells were isolated from LNCaP cell subcutaneous xenograft tumor of castrated mouse. C4-2-DR cells were then generated by acquired resistance to 20 μM Enzalutamide, an androgen-receptor antagonist.

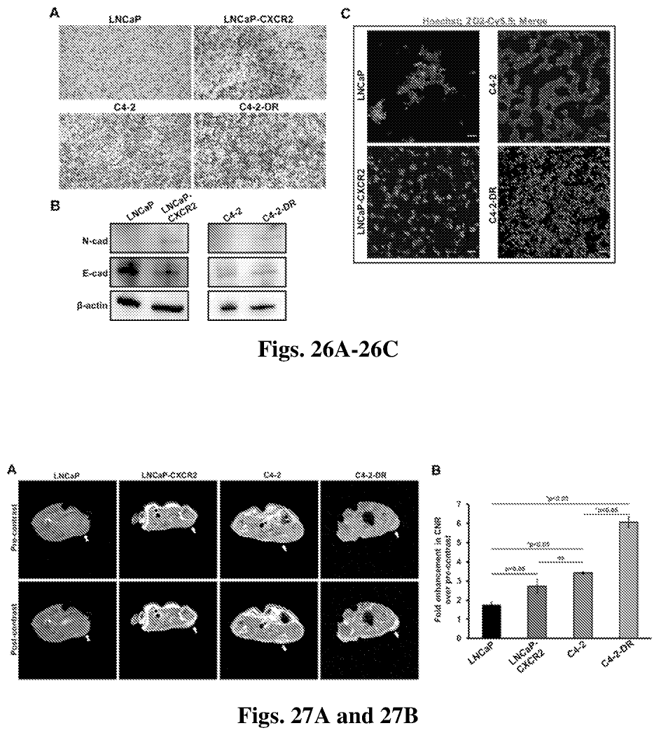

In standard transwell assays, compared to the low-risk indolent LNCaP cells, the LNCaP-CXCR2, C4-2, and C4-2-DR cells showed increased invasion through matrigel-coated inserts (FIG. 26A) and increase in EMT marker N-cadherin (FIG. 26B). In 3D culture, the invasive prostate cells showed proliferative tumor spheroids with significantly increased EDB-FN secretion, compared to LNCaP spheroids, evidenced by ZD2-Cy5.5 staining (FIG. 26C). These results indicate that EDB-FN is strongly upregulated in low-risk tumors that evolve into invasive tumors.

Xenograft models were established by subcutaneous flank injections in athymic nu/nu mice for the 4 prostate cancer cells and tested with MRMI using 0.04 mmol Gd/kg of MT218. Differential contrast enhanced MRMI of the tumors showed that MT218 results in stronger signal enhancement in the invasive LNCaP-CXCR2, C4-2, and C4-2-DR tumors, compared to the low-grade LNCaP tumors (FIG. 27A). This was reflected in the nearly 3-fold increase of CNR in the LNCaP-CXCR2 and C4-2 tumors and 6-fold increase in C4-2-DR tumors at 20 min post-injection, while the low-grade LNCaP tumors demonstrated negligible increase in their CNR (FIG. 27B).

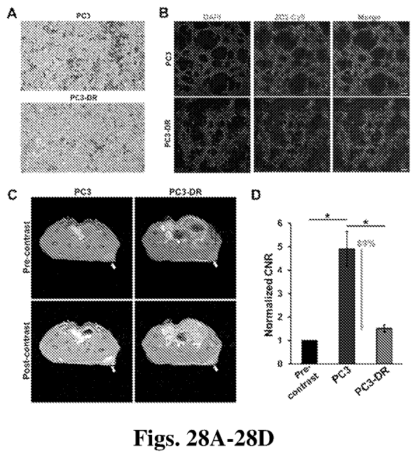

Interestingly, one additional model, PC3 and its drug-resistant counterpart PC3-DR showed a different trend. PC3-DR cells generated from PC3 cells by acquired resistance to 200 nM paclitaxel, showed decreased invasion through Matrigel-coated inserts (FIG. 28A), along with decreased EDB-FN secretion in 3D culture, as observed by the lower intensity of ZD2-Cy5.5 staining (FIG. 28B), compared to PC3 cells.

These results were also recapitulated in MRMI with MT218. As shown in FIG. 28C-D, while PC3 tumors showed robust signal enhancement and 5-fold higher CNR than the pre-contrast, the PC3-DR tumors only showed moderately enhanced signal enhancement and 1.6-fold CNR (p=0.07) over the pre-contrast. Importantly, the invasive PC3 tumors showed significantly higher CNR (3.3-fold) than the less invasive PC3-DR tumors (FIG. 28D), indicating that EDB-FN upregulation is associated only with invasive drug-resistant prostate cancer.

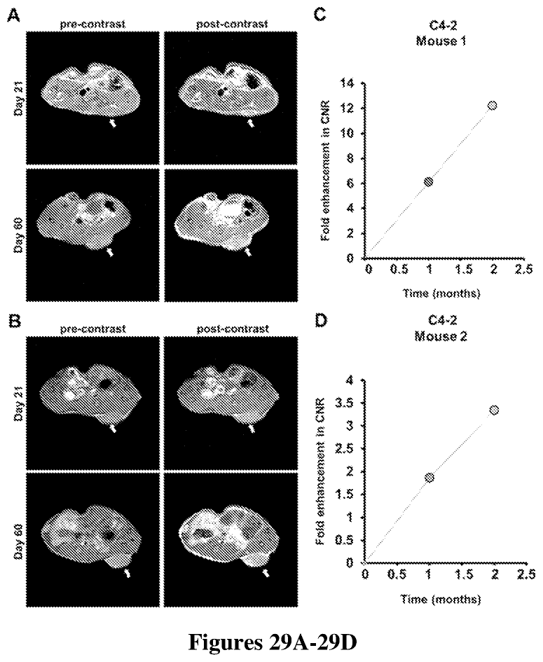

Preliminary research was also performed on active surveillance of prostate tumors over the course of 2 months. MRMI with MT218 was performed at day 21 and then at day 60 to monitor the progression of invasive C4-2 tumors. Two mice were imaged before and 20 min post-injection of 0.04 mmol Gd/kg MT218 and T₁-weighted axial images were obtained. As shown in FIG. 29, progression of C4-2 tumors at month 2 is accompanied by increased signal enhancement (FIG. 29A-B), reflected by steady increase in CNR from day 21 to day 60 (FIG. 29C-D).

### Example 6: The Efficacy of Targeted ECO/miR-200c Nanoparticle Treatment for Modulating Tumor Microenvironment and Treating Triple Negative Breast Cancer as Non-Invasively Determined by MR Molecular Imaging

This example describes the investigation of the effectiveness of MRMI for non-invasive assessment of tumor response to targeted miR-200c therapy and the therapeutic efficacy of RGD-PEG-ECO/miR-200c nanoparticles in mouse TNBC models. Targeted RGD-PEG-ECO/miR-200c nanoparticles were developed by self-assembly of miR-200c duplex with a multifunctional amino lipid carrier ECO and a cyclic RGD peptide with a PEG spacer for specific delivery and upregulation of miR-200c in TNBC cells. Previous research by the inventors using ECO, (1-aminoethyl)iminobis[N-oleicylcysteinyl-1-aminoethyl)propionamide], has shown superior efficiency for in vivo systemic delivery of therapeutic siRNAs and effective regulation of various oncogenic mRNAs and long non-coding RNAs for TNBC treatment. Parvani et al., Bioconjugate Chem., 30, 907 (2019). Because miR-200c directly regulates fibronectin expression, we first investigated the downregulation of its oncofetal subtype EDB-FN, along with other downstream gene targets, including ZEB1, BMI1, and FN1, in TNBC cancer cells following RGD-PEG-ECO/miR-200c treatment. The impact of RGD-PEG-ECO/miR-200c on spheroid formation, invasiveness, and migration of TNBC cancer cells was evaluated in vitro. Therapeutic efficacy and tumor response to systemic administration of RGD-PEG-ECO/miR-200c nanoparticles was investigated by non-invasive imaging of EDB-FN using MRMI with MT218 in two mouse models of orthotopic human TNBC and further validated by post-mortem immunohistochemistry.

### Results

### Targeted RGD-PEG-ECO/miR-200c Nanoparticles

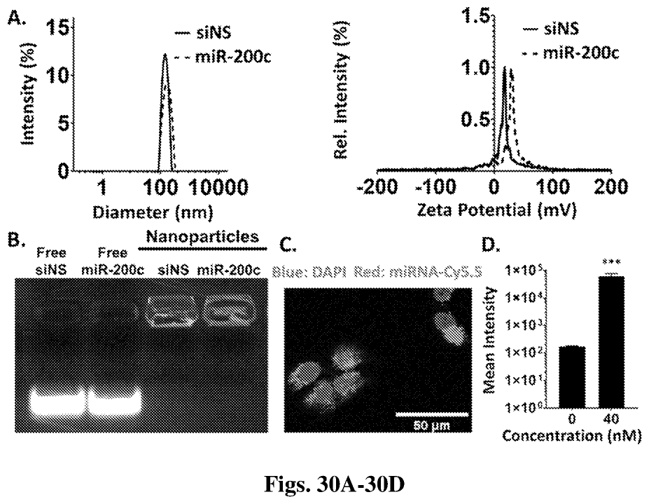

Cyclic RGD peptide-targeted RGD-PEG-ECO/miR-200c nanoparticles were formulated via self-assembly of RGD-PEG-MAL and ECO with unmodified miR-200c duplex (N/P=8), as described previously. Parvani et al., Bioconjugate Chem., 30, 907 (2019) The miR-200c duplex was used because the native single stranded miR-200c sequence had poor stability and was unable to form stable nanoparticles with ECO. RGD-PEG-ECO/siNS nanoparticles of a non-specific siRNA duplex were similarly prepared as control. RGD-PEG-ECO/miR-200c and RGD-PEG-ECO/siNS nanoparticles possessed uniform size and charge distribution with hydrodynamic diameters of 174.0±25.4 nm and 149.6±5.4 nm, and zeta potential of 25.3±4.4 mV and 17.7±2.4 mV, respectively, as measured by dynamic light scattering (FIG. 30A). Standard gel retardation assay exhibited efficient RNA entrapment and negligible free RNA bands for both the nanoparticles, indicating high RNA loading efficiency and encapsulation (FIG. 30B).

The cellular internalization of miR-200c was evaluated using RGD-PEG-ECO/miR-Cy5.5 nanoparticles bearing the miR-200c duplex labeled with Cy5.5 in the invasive, mesenchymal, miR-200c-low MDA-MB-231 TNBC cells. As shown in FIG. 30C, confocal fluorescence microscopy showed robust Cy5.5 fluorescence intensity around the nuclei at 4 h post-transfection, indicating efficient cytosolic delivery with the nanoparticles. Flow cytometry further confirmed the high efficiency of RGD-PEG-ECO/miR-Cy5.5 nanoparticle transfection with a 360-fold increase in fluorescence intensity as compared to non-transfected cells at 4 h post-transfection (FIG. 30D). These results suggest that the targeted RGD-PEG-ECO/miR-200c nanoparticles can effectively mediate efficient intracellular delivery of miR-200c in TNBC cells.

RGD-PEG-ECO/miR-200c Nanoparticles Facilitate Prolonged miR-200c Upregulation and Effective Regulation of Downstream Gene Targets in TNBC Cells

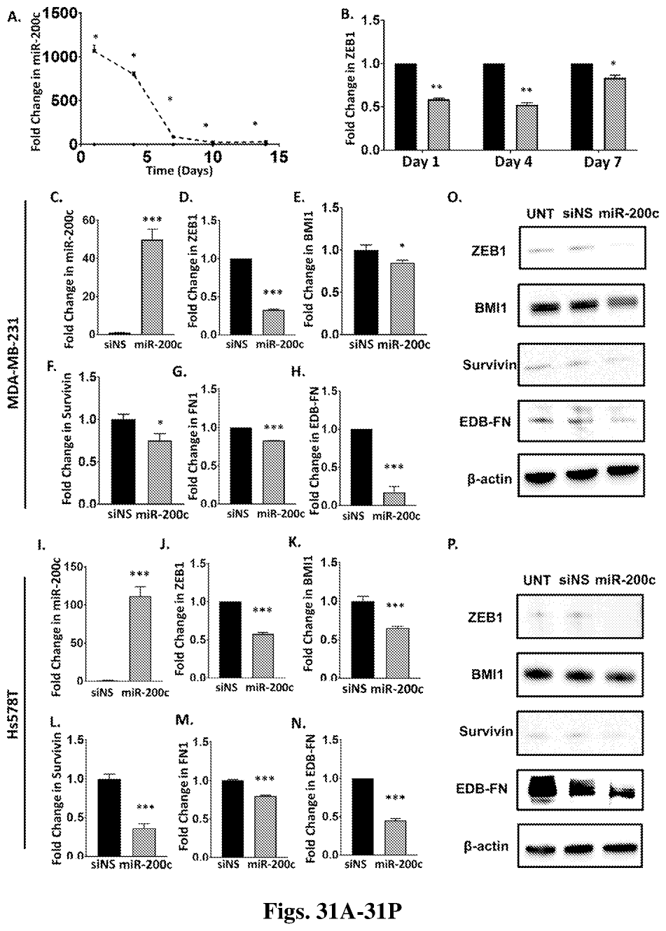

The effectiveness of RGD-PEG-ECO/miR-200c nanoparticles on intracellular overexpression and consequent regulation of downstream genes was investigated in MDA-MB-231 and Hs578T human TNBC cells. RGD-PEG-ECO/miR-200c nanoparticles mediated prolonged upregulation of miR-200c in MDA-MB-231 cells for up to 14 days post-transfection as determined by qRT-PCR, FIG. 31A. As compared to RGD-PEG-ECO/siNS, RGD-PEG-ECO/miR-200c nanoparticles induced nearly 1000-fold increase in miR-200c expression within the first 24 h post-transfection and maintained high levels for 4 days before a substantial decrease to 80-fold on day 7. A significant 30-fold upregulation of miR-200c was still observed up to day 14. The miR-200c upregulation mediated substantial downregulation of the mRNA expression of ZEB1, an EMT marker and direct target of miR-200c, in MDA-MB-231 cells. As shown in FIG. 31B, 20-40% downregulation of ZEB1 was observed for up to 1 week post-transfection of the RGD-PEG-ECO/miR-200c nanoparticles as compared to RGD-PEG-ECO/siNS. Similarly, treatment of both MDA-MB-231 and Hs578T cells with RGD-PEG-ECO/miR-200c nanoparticles resulted in a robust 50- and 100-fold upregulation of miR-200c, as compared to their respective control siNS counterparts at 48 h post-transfection as shown in FIG. 31C,I. The variances in miR-200c expression could be attributed to the number and rate of proliferation of the cells during transfection. Consequently, the expression of its direct downstream targets, including ZEB1, BMI1 and survivin, was significantly reduced at the mRNA level by 57% and 40% for ZEB1 (FIG. 31D,J), 15% and 35% for BMI1 (FIG. 31E,K), and 25% and 64% for survivin in MDA-MB-231and Hs578T, respectively (FIG. 31F,L). Substantial downregulation of these gene targets was also observed at protein levels by western blotting (FIG. 31O,P).

We next investigated the impact of RGD-PEG-ECO/miR-200c treatment on the expression of the extracellular matrix protein, fibronectin (FN1), which is also involved in EMT and is a direct target of miR-200c via3′-UTR binding-mediated repression of gene expression. RGD-PEG-ECO/miR-200c treatment resulted in an approximately 20% reduction in FN1 mRNA level in both MDA-MB-231 and Hs578T cells (FIG. 31G,M). The impact of miR-200c upregulation on the expression of the oncofetal isoform of FN1, EDB-FN, was also investigated to assess the potential of using EDB-FN as a marker for non-invasive imaging of the tumor response to miR-200c therapy. While both cell lines showed strong endogenous EDB-FN expression, it was much higher in Hs578T cells than in MDA-MB-231 cells (FIG. 31O,P). Approximately 80% and 40% reduction in EDB-FN mRNA level was observed in MDA-MB-231 and Hs578T cells (FIG. 31H,N), respectively, corroborated by decreased protein expression after the RGD-PEG-ECO/miR-200c treatment as compared to RGD-PEG-ECO/siNS via western blot (FIG. 31O,P), suggesting that EDB-FN is a downstream target of miR-200c. Taken together, these results demonstrate that the robust and prolonged intracellular delivery miR-200c by RGD-PEG-ECO/miR-200c can effectively regulate multiple downstream target genes of miR-200c in TNBC cells.

### RGD-PEG-ECO/miR-200c Nanoparticles Suppress Invasion and 3D Growth of TNBC Cells

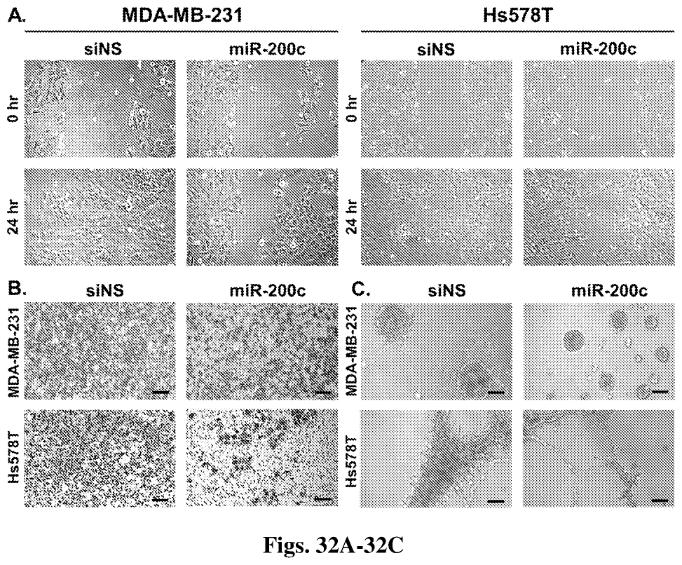

The impact of RGD-PEG-ECO/miR-200c mediated miR-200c upregulation to suppress the aggressiveness of TNBC cells was determined by using standard functional assays for migration, invasion, and tumor spheroid formation. Transfection of MDA-MB-231 and Hs578T cells with RGD-PEG-ECO/miR-200c reduced the migration of both cell lines as compared to RGD-PEG-ECO/siNS (FIG. 32A). The treatment with RGD-PEG-ECO/miR-200c for 24 h significantly reduced their ability to invade through Matrigel-coated porous membranes, as compared to RGD-PEG-ECO/siNS, FIG. 32B, where the invaded cells were stained purple with crystal violet. RGD-PEG-ECO/miR-200c significantly reduced the propensity of the TNBC cells to form tumor spheroids in 3D Matrigel culture when compared to the RGD-PEG-ECO/siNS control (FIG. 32C). These results demonstrate that the treatment with RGD-PEG-ECO/miR-200c suppress migration, invasion, and 3D spheroid formation of TNBC cells.

### RGD-PEG-ECO/miR-200c Suppresses In Vivo TNBC Proliferation

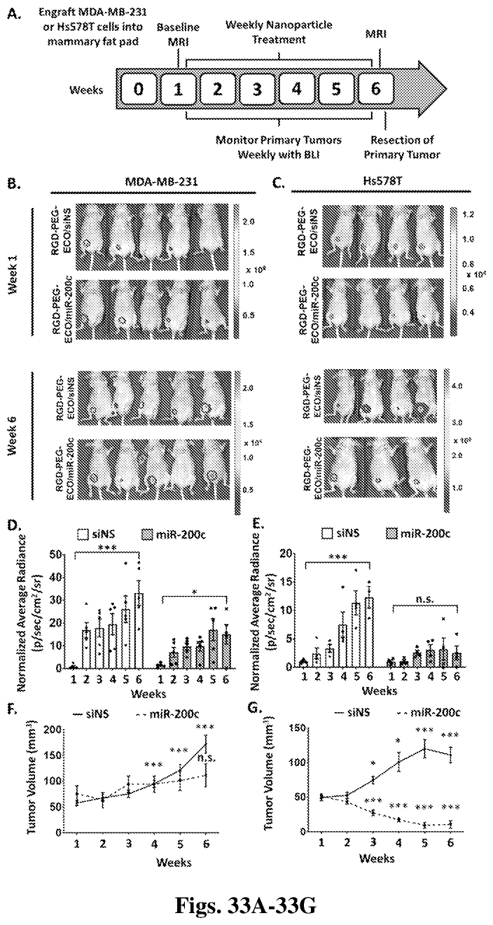

The efficacy of RGD-PEG-ECO/miR-200c nanoparticles for treating TNBC in vivo was evaluated in athymic nude mice bearing orthotopic GFP-Luciferase-labeled MDA-MB-231 and Hs578T xenografts. RGD-PEG-ECO/miR-200c and RGD-PEG-ECO/siNS (N/P=8) nanoparticles were formulated with 5% sucrose in aqueous solution, and intravenously administered weekly at a dose of 1.0 mg/kg RNA for 6 weeks (FIG. 33A). Tumor growth was monitored using caliper measurements and whole body bioluminescence imaging (BLI) weekly and with MRMI before and at the end of the treatment. The treatment with RGD-PEG-ECO/miR-200c resulted in a significant inhibition of tumor progression as compared to RGD-PEG-ECO/siNS in both the tumor models. RGD-PEG-ECO/siNS-treated mice showed substantially increased bioluminescence intensities at the week 6 compared to those before the treatment (week 1), indicating rapid tumor growth in the control group. In contrast, the miR-200c-treated tumors showed significantly less increase in signal intensity as compared to the control at week 6, FIG. 33B-E. The MDA-MB-231 tumors treated with RGD-PEG-ECO/miR-200c inhibited tumor progression with no significant increase in tumor volume from week 1 to week 6 (76.1±15.2 to 111.4±22.1 mm3, p>0.05), while the control-treated tumors had nearly 3-fold increase in tumor volume (57.7±32.0 to 172.2±17.8 mm³, p<0.005), FIG. 33F. In the case of Hs578T tumors, RGD-PEG-ECO/miR-200c treatment showed substantial reduction of tumor volume, from 51.48±3.37 to 10.54±5.45 mm3 (p<0.005), approximately 79% reduction from week 1 to week 6, while RGD-PEG-ECO/siNS-treated Hs578T tumors had a 2.2-fold increase in tumor volume (49.5±4.7 to 111.1±11.3 mm3, p<0.005), FIG. 33G. One mouse from RGD-PEG-ECO/miR-200c treatment, however, perished from unknown cases not linked to either therapy or tumor burden. The results indicate that the systemic delivery of miR-200c with tumor-targeting RGD-PEG-ECO/miR-200c nanoparticles mediates effective therapy of TNBC in animal models.

MRMI Demonstrates Altered EDB-FN Expression in TME after Treatment with RGD-PEG-ECO/miR-200c

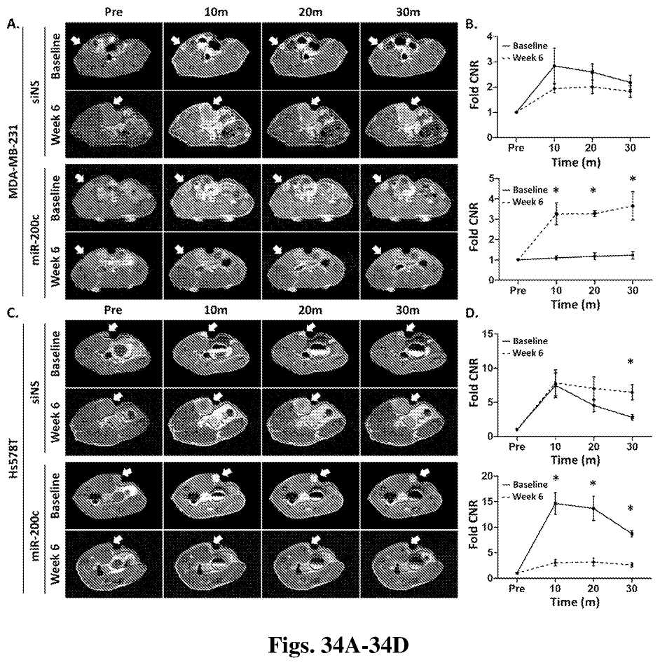

Given that miR-200c upregulation significantly downregulated EDB-FN expression in the TNBC cells in vitro and suppressed tumor proliferation in vivo, we sought to evaluate the tumor response to RGD-PEG-ECO/miR-200c treatment based on EDB-FN expression using high-resolution MRMI with MT218. FIG. 34 shows the T₁-weighted axial MR images of the TNBC tumors before and after treatment with RGD-PEG-ECO/miR-200c and RGD-PEG-ECO/siNS nanoparticles. Strong signal enhancement was observed in all the pretreatment MDA-MB-231 and Hs578T tumors in the first 30 min after injection of MT218 at a dose of 0.1 mmol/kg, when compared to the pre-contrast tumor images, indicating high expression of EDB-FN in the ECM of both the tumor models, since MT218 is an extracellular contrast agent. After 6 weeks of treatment, strong signal enhancement remained in the post-contrast MR images of the MDA-MB-231 and Hs578T tumors that received control RGD-PEG-ECO/siNS. MRMI with MT218 clearly delineated significant growth of both the tumor xenografts in the control groups. In contrast, no significant signal enhancement was seen in the post-contrast MR images of the 2 tumor models treated with RGD-PEG-ECO/miR-200c, when compared to the pretreatment tumors and the control-treated tumors, indicating that the miR-200c treatment resulted in significant reduction of EDB-FN in the TNBC tumors. Similar to the observation in FIG. 34, MRMI revealed inhibition of MDA-MB-231 tumor proliferation and the regression of the Hs578T tumors after treatment with RGD-PEG-ECO/miR-200c, as compared to their respective control-treated tumors. These results indicate that RGD-PEG-ECO/miR-200c treatment reduces EDB-FN expression in tumor ECM and alters the TME of the TNBC tumors as shown by non-invasive MRMI with MT218.

Quantitative analysis of contrast-to-noise ratio (CNR) in the MR images of the tumors revealed high CNR in both MDA-MB-231 and Hs578T tumors before treatment with both the nanoparticles (baseline) after intravenous injection of MT218, indicating high EDB-FN expression, FIG. 34A. The post-contrast CNR in the Hs578T tumors was substantially higher than in the MDA-MB-231 tumors, indicating higher EDB-FN expression in the former, consistent to the western blotting of EDB-FN in the cells (FIG. 31O,P). The treatment with RGD-PEG-ECO/miR-200c significantly decreased the CNR in both MDA-MB-231 and Hs578T tumors, indicating decrease of EDB-FN expression, while the tumors treated with RGD-PEG-ECO/siNS had the same high CNR as the pretreated tumors in the same group of mice. The post-contrast CNR in the miR-200c-treated MDA-MB-231 tumors showed no significant increase compared to the pre-contrast background level, while the treated Hs578T tumors was slightly higher CNR than the background level (FIG. 34B). This observation suggested more residual EDB-FN in Hs578T tumors than in MDA-MB-231 tumors after miR-200c treatment, which is also consistent with western blotting results of EDB-FN in the miR-200c-treated cells (FIG. 31O,P). Taken together, these results suggest that MRMI with MT218 provide non-invasive and accurate visualization of tumor response to the treatment of RGD-PEG-ECO/miR-200c nanoparticles based on EDB-FN expression, and effective assessment of the therapeutic efficacy of miR-200c therapy.

RGD-PEG-ECO/miR-200c Therapy Elevates miR-200c Levels and Suppresses Downstream Target Genes in TNBC Tumors

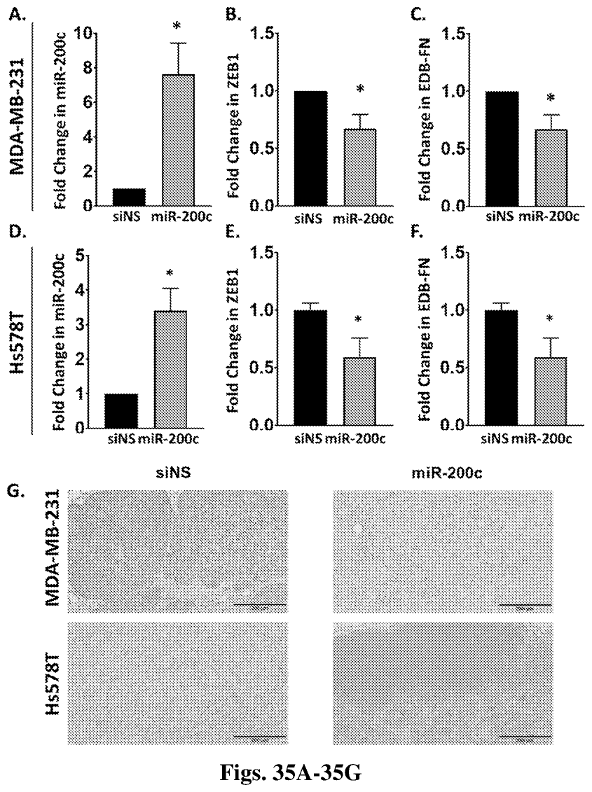

Systemic administration of RGD-PEG-ECO/miR-200c nanoparticles resulted in robust upregulation of miR-200c in the TNBC tumors, as shown by qRT-PCR analysis of the surgically resected primary tumors. MDA-MB-231 and Hs578T tumors treated with RGD-PEG-ECO/miR-200c had approximately 7.6-fold and 3.4-fold increase of miR-200c levels, respectively, as compared to those treated with RGD-PEG-ECO/siNS, FIG. 35A,D. Importantly, this miR-200c upregulation led to significant downregulation of ZEB1 and EDB-FN at the mRNA level, FIG. 35B,C,E,F. The downregulation of EBF-FN in the RGD-PEG-ECO/miR-200c-treated tumors was also demonstrated by immunohistochemistry. Strong EDB-FN staining was observed in the tissue sections of the RGD-PEG-ECO/siNS-treated MDA-MB-231 and Hs578T tumors, as stained by an anti-EDB-FN antibody, while the tumors treated with RGD-PEG-ECO/miR-200c exhibited substantial decrease in EDB-FN staining (FIG. 35G), consistent to the observation with non-invasive MRMI. Taken together, these results indicate that systemic treatment of TNBC tumors with RGD-PEG-ECO/miR-200c can effectively upregulate their miR-200c expression and, consequently downregulate the downstream oncogenes, ZEB1 and EDB-FN, in vivo. The altered levels of miR-200c and their related downstream genes in the tumors of the animal models are consistent to the data obtained in the in vitro experiments and strongly corroborate the tumor responses to RGD-PEG-ECO/miR-200c treatment.

RGD-PEG-ECO/miR-200c does not Cause Overt Toxic Side-Effects in Mice

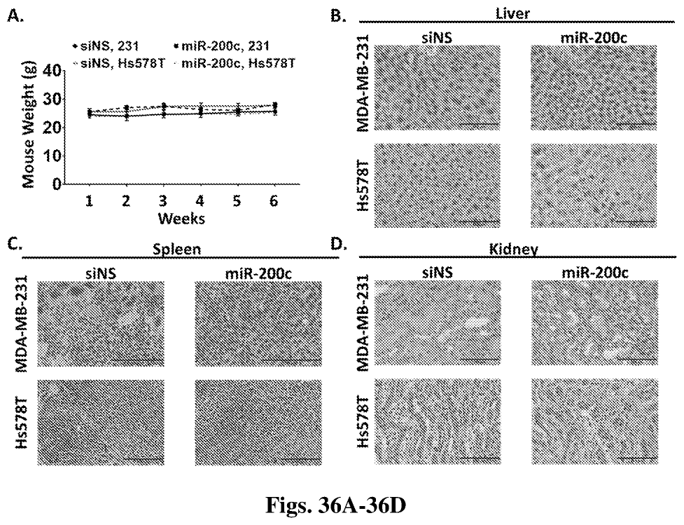

The safety of repeated systemic administration of RGD-PEG-ECO/miR-200c- and RGD-PEG-ECO/siNS nanoparticles was assessed by monitoring the body weights of the mice during the treatment. No significant changes in body weights were observed in any of the mouse cohorts during 6 weeks of repeated nanoparticle injections, FIG. 36A. Histopathological analysis with H&E staining of the liver, spleen, and kidneys, which are involved in the clearance of the nanoparticles, demonstrated normal histology without any overt tissue damage in the mice treated with both the nanoparticles. These results indicate excellent safety of systemic administration RGD-PEG-ECO/miR-200c nanoparticles for targeted therapy of TNBC.

### Discussion

This work demonstrates, for the first time, non-invasive imaging of tumor response to targeted miR-200c therapy with MR molecular imaging of EDB-FN in TME of TNBC. Fibronectin (FN1) is considered as one of the EMT markers and is an important ECM protein in cancerous tissues. However, it is also present in the ECM of normal tissues, which precludes its clinical value as a suitable oncomarker. Its oncofetal isoform EDB-FN is specifically overexpressed in aggressive tumors and absent in normal tissues, thus making it a promising target for cancer imaging and therapy. Kumra, D. P. Reinhardt, Advanced Drug Delivery Reviews, 97, 101 (2016). Both MDA-MB-231 and Hs578T TNBC cells have low miR-200c expression and high EDB-FN expression. The miR-200c upregulation mediated by RGD-PEG-ECO/miR-200c nanoparticles resulted in a significant downregulation of EDB-FN in both the cell lines in correlation with suppression of their invasion in vitro. Similarly, weekly systemic injections of RGD-PEG-ECO/miR-200c nanoparticles in mice significantly suppressed the proliferation of MDA-MB-231 and Hs578T TNBC tumors in vivo as compared to the non-specific control RGD-PEG-ECO/siNS. In pre-treatment MRMI scans with MT218, both the tumor models showed strong signal enhancement, which was significantly reduced in the scans of both the miR-200c-treated tumors, but not in the images of the control-treated tumors. These observations suggest that miR-200c influences EDB-FN expression, either directly or indirectly, and that MRMI of therapy-induced changes in EDB-FN levels can effectively provide non-invasive assessment of tumor response to RGD-PEG-ECO/miR-200c treatment.

MRMI with MT218 also demonstrates for the first time that targeted delivery of miR-200c with RGD-PEG-ECO/miR-200c effectively alters the tumor microenvironment of TNBC. FN1 and EDB-FN are ECM proteins that play important roles in through facilitating migration and invasion of cancer cells and mediating their interactions with the stroma. Vaidya et al., Cells, 9, 1826 (2020). MRMI non-invasively provides three-dimensional, high-resolution images of EDB-FN expression levels throughout the tumors before and after the miR-200c treatment. The reduced MRMI signal in the images of the tumors treated with RGD-PEG-ECO/miR-200c indicates downregulation of EDB-FN, confirmed through post-mortem immunohistochemistry of the tumor sections. These results suggest that upregulation of miR-200c could alter the TME in both the TNBC tumor models by regulating the oncoprotein EDB-FN and consequent tumor ECM remodeling. The impact of miR-200c expression on the interplay between several other components of the TME, including collagens, laminins, and immune circuitry, and their consequent modulation of the therapeutic outcomes would need additional detailed research.

In regard to the miR-200c therapy, while both the TNBC tumor models exhibited similar biological response in terms of EDB-FN expression revealed by MRMI, they also showed model-specific differences to RGD-PEG-ECO/miR-200c nanoparticles. The MDA-MB-231 tumors became static with no significant size changes during the treatment, whereas significant regression was observed in Hs578T tumors after 6-week treatment. Post-treatment analysis showed significant upregulation of miR-200c and downregulation of ZEB1 and EDB-FN in both the treated tumor models. Because miR-200c targets numerous other oncogenes regulating pro-cancerous pathways including Notch, Hedgehog, and Wnt, the discrepancy in tumor size between the two treated tumor models could be partly explained by the here-to-fore untested molecular pathways directly influenced by miR-200c upregulation. Further comprehensive investigations are needed to understand such and other differences between biological response and therapeutic outcome based on the tumor size in multiple TNBC models. Nevertheless, the ability of RGD-PEG-ECO/miR-200c to directly regulate numerous oncogenes suggests that miR-200c is a promising therapy for effective treatment of heterogenous TNBC tumors. This work provides strong evidence that miR-200c could be a promising therapeutic target for safe treatment of aggressive TNBC tumors through systemic delivery of targeted RGD-PEG-ECO/miR-200c nanoparticles. The combination of MRMI with MT218 and RGD-PEG-ECO/miR-200c therapy could allow for the non-invasive and longitudinal assessment of tumor response to achieve precision healthcare of cancer patients.

In summary, we have demonstrated the effectiveness of MRMI of EDB-FN for non-invasive assessment of tumor response to RGD-PEG-ECO/miR-200c nanoparticle therapy. Weekly administration of RGD-PEG-ECO/miR-200c at a relatively low dose resulted in inhibition and regression of TNBC tumors in mouse models without overt toxic side-effects. MRMI with MT218 demonstrated downregulation of tumor-specific EDB-FN levels and altered tumor microenvironment following miR-200c upregulation, and is a promising molecular imaging modality for non-invasive assessment of tumor response to cancer treatment. This research opens new avenues for developing novel precision medicine with the combination of molecular imaging and systemic regulation of miRNA levels for personalized treatment of TNBC patients.

### Experimental Section

### Cells and Cell Culture

Triple-negative breast cancer lines, MDA-MB-231 and Hs578T, were purchased from ATCC (Manassas, VA). MDA-MB-231 were maintained in Dulbecco's Modified Eagle's Medium (DMEM, Gibco) supplemented with 10% fetal bovine serum (FBS, Gibco) and 1% Penicillin/Streptomycin. Hs578T cells were cultured in DMEM supplemented with 10% FBS, 1% Penicillin/Streptomycin, and 0.01 mg/mL recombinant human insulin from Sigma-Aldrich (St. Louis, MO). All the cells were cultured in a humidified incubator kept at 37° C. and 5% CO₂. The cell lines were engineered to express firefly luciferase and GFP with the lentivirus, CMV-Luciferase (Firefly)-2A-GFP (Neo), from Amsbio (Cambridge, MA) and sorted for selection using flow cytometry.

### Nanoparticle Formulation

The amino lipid ECO (MW=1023) and the targeting ligand RGD-PEG-MAL (PEG, 3.4 k, Creative PEGworks, Durham, NC) were synthesized. miR-200c duplex and negative control siRNA (siNS) were dissolved in nuclease-free water to a concentration of 25 μmol/L. Fresh RGD-PEG-MAL was dissolved in NF water to a stock concentration of 0.625 mmol/L. ECO was dissolved in 100% ethanol to a stock concentration of 5 and 50 mM for in vitro and in vivo experiments. Targeted nanoparticle formulations were prepared by first mixing RGD-PEG-MAL (2.5 mol %) with ECO in nuclease-free water for 30 min with gentile agitation. This was followed by complexation with miR-200c or siNS duplexes to form the corresponding targeted ECO/miR-200c or ECO/siNS nanoparticles at N/P=8. For in vivo experiments, nanoparticle formulations were mixed with 5% sucrose, flash frozen and stored in −80° C.

### Nanoparticle Characterization

Nanoparticle diameter and zeta potential were measured upon dilution in NF water (1:20) using a Litesizer 500 from Anton Paar GmbH (Graz, Austria) at 25° C. Transmission electron microscopy (TEM) was conducted by loading nanoparticles (20 μL) onto a copper grid coated with a thin carbon film (20 nm), and stained with 3 μL of 2% uranyl acetate solution. Samples were imaged via TEM after drying. Encapsulation of the RNA cargo was assessed using agarose gel electrophoresis, where 20 μL of nanoparticles were mixed with 4 μL loading dye from Roche (Basel, Switzerland) and loaded onto a 1% agarose gel containing ethidium bromide. Electrophoresis was done at 100 V for 30 min, and the bands were visualized using ChemiDoc™ XRS+ Imager (BioRad, Hercules, CA).

### Cellular Uptake of ECO/miRNA Nanoparticles

Qualitative assessment of cellular uptake of RGD-PEG-ECO/miRNA nanoparticles was evaluated using live cell fluorescence microscopy. MDA-MB-231 cells (100,000 cells) were seeded onto a glass-bottom microwell dish, allowed to adhere overnight, and then treated with RGD-PEG-ECO/miRNA-Cy5.5 (Dharmacon, Lafayette, CO) at N/P=8 and a [miRNA]=40 nM. After 4 h, the transfection media was replaced with media containing 5 μg/mL Hoechst 33342 (Thermo Fisher Scientific, Waltham, MA) for 30 min. Cells were subsequently washed with DPBS and imaged using an Olympus FV1000 confocal microscope. Cellular uptake was similarly evaluated quantitatively using flow cytometry. Transfected cells were harvested with 0.25% trypsin containing 0.26 mmol/L EDTA (Invitrogen, Carlsbad, CA), centrifuged at 12,000 rpm for 5 min, and fixed in 5% paraformaldehyde in DPBS (500 μL). After filtration through a 35-μm cell strainer (BD Biosciences, San Jose, CA), cellular uptake was quantified by measuring the fluorescence intensity of Cy5.5 using a BD FACSCalibur flow cytometer.

### Semi-Quantitative RT-PCR Analyses

Semiquantitative real-time PCR was conducted. Briefly, total RNA, including miRNAs, was extracted from cells and tissues using the RNeasy Plus Mini Kit from Qiagen (Germantown, MD) as per manufacturer's instructions. Reverse transcription was performed using the miScript II RT Kit (Qiagen, Hilden, Germany), followed by qPCR using miScript SYBR Green PCR kit (Qiagen), containing human RNU6B (RNU6-2) miScript Primer Assay and miScript Universal Primer. miRNA and mRNA expression levels were normalized to U6 (Qiagen) and 18S controls respectively.

### Immunoblotting Analyses

Total protein was extracted by lysing cell pellets (1:1 protease inhibitor in PBS and Laemmli buffer). The lysates were incubated at 100° C. for 10 min and centrifuged at 13,200 rpm at 4° C. for 15 min. The protein concentration was determined using Lowry assay kit, according to the manufacturer's protocol (Bio-Rad). Equal amounts of protein extracts (40 μg) were separated with SDS-PAGE and then transferred onto a nitrocellulose membrane. Membranes were blocked for 1 h with 5% BSA in TBST. The following antibodies were used (1:1000 dilution, overnight incubation at 4° C.): anti-ZEB1, anti-BMI1, anti-Survivin, anti-Vimentin, and anti-R-actin monoclonal antibodies (Cell Signaling Technology, Danvers, MA) as well as anti-EDB-FN G4 clone (Absolute Antibody, UK). Following secondary antibody incubation (1:2000 dilution for 1 h), the membranes were developed with Signal Fire Plus ECL Kit (Cell Signaling Technology) and imaged on ChemiDoc™ XRS+ Imager (Bio-Rad).

### Scratch Wound Assay

Scratch wounds were performed. Briefly, 1×10⁶ breast cancer cells were plated on 6-well plates for 24 hours. A 10 μL pipet tip was used to make a single scratch in the center of the plate. Cells were washed with DPBS, transfected with RGD-PEG-ECO/miR-200c or RGD-PEG-ECO/siNS nanoparticles at [RNA]=100 nM, and monitored for 24 h until the wounds closed. Images were taken using a Motiram T₂ camera (Motic, Xiamen, China).

### Transwell Migration Assay

Transwell migration assays were performed. TNBC cells were transfected with targeted ECO/miR-200c and ECO/siNS nanoparticles at [RNA]=100 nM for 48 h. Serum-starved cells were seeded onto Transwell inserts (50,000 cells/insert) (VWR, Radnor, PA) coated with 0.28 mg/mL Corning Matrigel Membrane Matrix (Corning, NY). After 24 h, the inserts were swabbed to remove non-migratory cells. The migrated cells on the bottom of the Transwell inserts were fixed in 10% formalin (10 min) and stained with 0.1% crystal violet (20 min). Images were obtained using a Moticam T₂ camera.

### 3D Tumor Spheroid Formation Growth Assay

Matrigel growth assay was conducted. TNBC cells were treated with REG-PEG-ECO/miR-200c or RGD-PEG-ECO/siNS nanoparticles at 100 nM RNA for 48 h. Cells were then plated onto Corning Matrigel Membrane Matrix-coated μ-Slides (8-well, Ibidi, Grafelfing, Germany) at a density of 100,000 cells/well. The tumor spheroids were evaluated for up to 48 h and imaged using the Moticam T₂ camera.

### Animal Models

Four to six week old, female nude mice (athymic nu/nu background) were purchased from Jackson Laboratories (Bar Harbor, Maine) and cared for in the Animal Core Facility at Case Western Reserve University (Cleveland, OH). All animal experiments were conducted in accordance with an approved protocol by the IACUC of CWRU. Mammary fat pads of the mice were injected with 2×10⁶ GFP-Luc-labeled MDA-MB-231 or 4×10⁶ Hs578T cells suspended in a matrigel-PBS mixture. Tumor volumes were monitored weekly using a Vernier caliper and bioluminescence imaging (BLI). The mice that reached an average tumor volume of 50 mm³, were randomized into control and treated groups (n=5/cohort for MDA-MB-231, and n=4/cohort for Hs578T, 2 mice did not present tumors).

### In Vivo Nanoparticle Therapy

The mice bearing orthotopic MDA-MB-231 and Hs578T tumors were intravenously injected with in vivo formulations of RGD-PEG-ECO/miR-200c or RGD-PEG-ECO/siNS nanoparticles (1 mg/kg, 5% w/v sucrose) once a week for 6 weeks (1 mg/kg, 5% w/v sucrose). Primary tumor growth was monitored via caliper measurements and BLI once a week. After 6 weeks, the primary tumors were surgically resected and analyzed for histology, RNA expression, and IHC. In addition, the spleen, liver, and kidneys were extracted for histology. A portion of the tumor was fixed in 10% neutral buffered formalin (Sigma-Aldrich), followed by paraffin embedding, sectioning, and H&E staining. IHC was performed using anti-EDB-FN G4 antibody. Staining service was provided by the Tissues Resources Core Facility of CWRU. One RGD-PEG-ECO/miR-200c treated Hs578T tumor-bearing mouse had to be sacrificed, but was analyzed for cause of death prior for tumor metastases and therapeutic toxicity through histology, both revealing none.

### MRMI-Mediated Monitoring of Therapeutic Response

The targeted MRI contrast agent ZD2-N3-Gd(HP-DO3A) (MT218) was provided by Molecular Theranostics (Cleveland, OH) and was synthesized. MR images of the mice bearing MDA-MB-231 or Hs578T tumors were acquired on a 3T MRS 3000 scanner (MRS Solutions, Surrey, UK) with a mouse short quad coil. T₁-weighted images were acquired following intravenous injection of MT218 at dose of 0.1 mmol/kg using a fast spin echo (FSE) axial sequence with respiratory gating (TR=305 ms, TE=11 ms, FA=90°, FOV=40×40 mm, slice thickness=1 mm, slice number=15, Nav=2, matrix=256×256). Images were acquired before and at 10, 20, and 30 min after injection of MT218. MRMI was performed before the treatment and then at week 6 post-nanoparticle treatments for MDA-MB-231 and Hs578T tumors. MRI data and image processing was conducted using FIJI (FIJI is just ImageJ) software. The contrast-to-noise ratios (CNRs) of tumors were calculated as the difference of the mean tumor intensity and the mean muscle intensity divided by the standard deviation of the noise.

### Statistical Analyses

All the experiments were independently conducted at least 3 times (n=3). Statistical analysis was conducted using GraphPad software (v8.4.3) and p<0.05 was considered to be statistically significant. Data between 2 groups was compared using unpaired t-test. Data represented by more than 2 groups was compared with one-way ANOVA. The data is represented as mean±s.e.m. Dots represent individual mice or technical replicates as stated in the figure legends.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of detecting drug-resistant cancer in a subject, comprising contacting a tissue of the subject with an effective amount of a molecular probe comprising the formula P-L-C, wherein:
P is a peptide that includes an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9;
C is a contrast agent; and
L is a non-peptide linker that covalently links the peptide to the contrast agent, the linker including a carboxylic acid that forms a carboxamide with an amine of the peptide or a maleimide that forms a thioester bond with a cysteine reside of the peptide or a maleimide that forms a thioester with a cysteine residue of the peptide;
detecting the amount of the molecular probe present in the tissue;
comparing the amount of molecular probe detected to a control value; and
detecting drug-resistant cancer in the subject if the amount of the molecular probe present in the tissue is higher than the control value.

2. The method of claim 1, wherein the cancer is breast cancer, oral cancer, pancreatic cancer, or prostate cancer.

3. The method of claim 1, wherein the molecular probe is a magnetic resonance imaging agent.

4. The method of claim 1, wherein the tissue is contacted in vivo.

5. The method of claim 1, wherein the molecular probe is systemically administered to a subject having or suspected of having cancer.

6. The method of claim 1, wherein the non-peptide linker of the molecular probe is a non-peptide aliphatic or heteroaliphatic linker.

7. The method of claim 1, wherein the non-peptide linker of the molecular probe includes an alkylene dicarboxamide when covalently linking the peptide and contrast agent.

8. The method of claim 1, wherein the contrast agent of the molecular probe includes at least one of metal chelating agent or a metallofullerene.

9. The method of claim 1, wherein the contrast agent of the molecular probe includes a metal chelating agent comprising at least one of diethylenetriaminepentaacetate (DTPA) or its derivatives, 1,4,7,10-tetraazadodecanetetraacetate (DOTA) and its derivatives, 1,4,7,10-tetraazadodecane-1,4,7-triacetate (DO3A) and its derivatives, ethylenediaminetetraacetate (EDTA) and its derivatives, 1,4,7,10-tetraazacyclotridecanetetraacetic acid (TRITA) and its derivatives, 1,4,8,11-tetraazacyclotetradecane-1,4,8,11-tetraacetic acid (TETA) and its derivatives, 1,4,7,10-tetraazadodecanetetramethylacetate (DOTMA) and its derivatives, 1,4,7,10-tetraazadodecane-1,4,7-trimethylacetate (DO3MA) and its derivatives, N,N′,N″,N′″-tetraphosphonatomethyl-1,4,7,10-tetraazacyclododecane (DOTP) and its derivatives, 1,4,7,10-tetraazacyclododecane-1,4,7,10-tetrakis(methylene methylphosphonic acid) (DOTMP) and its derivatives, 1,4,7,10-tetraazacyclododecane-1,4,7,10-tetrakis(methylene phenylphosphonic acid) (DOTPP) and its derivatives, or N,N′-ethylenedi-L-cysteine and its derivatives.

10. The method of claim 1, wherein the molecular probe has the formula: 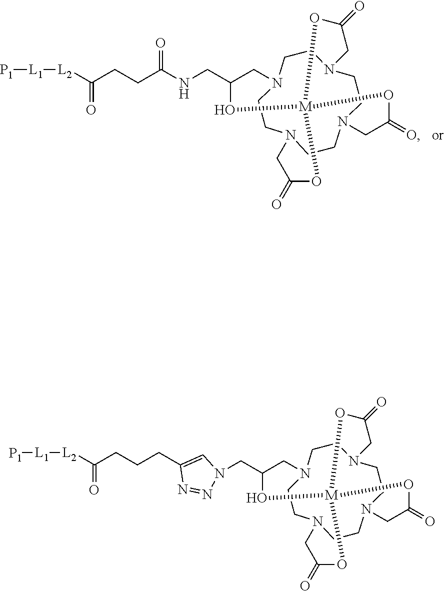
wherein: P₁ is a peptide having an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9;
L₁ is an optional spacer;
L₂ is an amino group of the peptide P₁ or the spacer; and
M is a metal selected from the group consisting of Gd⁺³, Eu⁺³, Tm⁺³, Dy⁺³, Yb⁺³, Mn⁺², Fe⁺³, ⁵⁵Co, ⁶⁴Cu, ⁶⁷Cu, ⁴⁷Sc, ⁶⁶Ga ⁶⁸Ga, ⁹⁰Y, ⁹⁷Ru, ⁹⁹mTc, ¹¹¹h, ¹⁰⁹Pd, ¹⁵3Sm, ¹⁷⁷Lu, ¹⁸⁶Re, and ¹⁸⁸Re; or salts thereof.

11. The method of claim 10, wherein L₁ comprises at least one of polyalkyleneoxide, polyvinyl alcohol, polyethylene glycol (PEG), polypropylene glycol (PPG), co-poly (ethylene/propylene) glycol, polyoxyethylene (POE), polyurethane, polyphosphazene, polysaccharides, dextran, polyvinylpyrrolidones, polyvinyl ethyl ether, polyacryl amide, polyacrylate, or polycyanoacrylates.

12. A method of monitoring the treatment of drug resistant cancer, comprising: contacting a tissue of a subject undergoing treatment of drug resistant cancer with an effective amount of a molecular probe comprising the formula P-L-C for a first time, wherein:
P is a peptide that includes an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9;
C is a contrast agent; and
L is a non-peptide linker that covalently links the peptide to the contrast agent, the linker including a carboxylic acid that forms a carboxamide with an amine of the peptide or a maleimide that forms a thioester bond with a cysteine reside of the peptide or a maleimide that forms a thioester with a cysteine residue of the peptide;
detecting a first amount of the molecular probe present in the tissue;
contacting the tissue of the subject for a second time with an effective amount of a molecular probe comprising the formula P-L-C;
detecting a second amount of the molecular probe present in the tissue; and
comparing the first amount and the second amount of the molecular probe to monitor the treatment of the drug resistant cancer in the subject.

13. The method of claim 12, wherein the cancer is breast cancer, oral cancer, pancreatic cancer, or prostate cancer.

14. The method of claim 12, wherein the molecular probe is a magnetic resonance imaging agent.

15. The method of claim 12, wherein the tissue is contacted in vivo.

16. The method of claim 12, wherein the drug resistant cancer is being treated with a chemotherapeutic agent.

17. The method of claim 12, wherein the non-peptide linker of the molecular probe is a non-peptide aliphatic or heteroaliphatic linker.

18. The method of claim 12, wherein the non-peptide linker of the molecular probe includes an alkylene dicarboxamide when covalently linking the peptide and contrast agent.

19. The method of claim 12, wherein the contrast agent of the molecular probe includes at least one of metal chelating agent or a metallofullerene.

20. The method of claim 12, wherein the contrast agent of the molecular probe includes a metal chelating agent comprising at least one of diethylenetriaminepentaacetate (DTPA) or its derivatives, 1,4,7,10-tetraazadodecanetetraacetate (DOTA) and its derivatives, 1,4,7,10-tetraazadodecane-1,4,7-triacetate (DO3A) and its derivatives, ethylenediaminetetraacetate (EDTA) and its derivatives, 1,4,7,10-tetraazacyclotridecanetetraacetic acid (TRITA) and its derivatives, 1,4,8,11-tetraazacyclotetradecane-1,4,8,11-tetraacetic acid (TETA) and its derivatives, 1,4,7,10-tetraazadodecanetetramethylacetate (DOTMA) and its derivatives, 1,4,7,10-tetraazadodecane-1,4,7-trimethylacetate (DO3MA) and its derivatives, N,N′,N″,N′″-tetraphosphonatomethyl-1,4,7,10-tetraazacyclododecane (DOTP) and its derivatives, 1,4,7,10-tetraazacyclododecane-1,4,7,10-tetrakis(methylene methylphosphonic acid) (DOTMP) and its derivatives, 1,4,7,10-tetraazacyclododecane-1,4,7,10-tetrakis(methylene phenylphosphonic acid) (DOTPP) and its derivatives, or N,N′-ethylenedi-L-cysteine and its derivatives.

21. The method of claim 12, wherein the molecular probe has the formula: 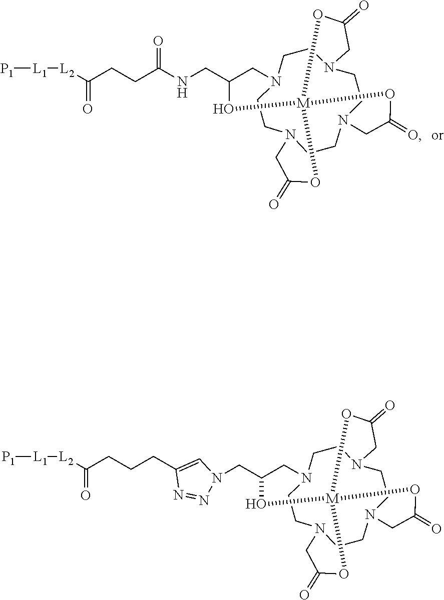
wherein: P₁ is a peptide having an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9;
L₁ is an optional spacer;
L₂ is an amino group of the peptide P₁ or the spacer; and
M is a metal selected from the group consisting of Gd⁺³, Eu⁺³, Tm⁺³, Dy⁺³, Yb⁺³, Mn⁺², Fe⁺³, ⁵⁵Co, ⁶⁴Cu, ⁶⁷Cu, ⁴⁷Sc, ⁶⁶Ga, ⁶⁸Ga, ⁹⁰Y, ⁹⁷Ru, ⁹⁹mTc, ¹¹¹h, ¹⁰⁹Pd, ¹⁵³Sm, ¹⁷⁷Lu, ¹⁸⁶Re, and ¹⁸⁸Re; or salts thereof.

22. The method of claim 21, wherein L₁ comprises at least one of polyalkyleneoxide, polyvinyl alcohol, polyethylene glycol (PEG), polypropylene glycol (PPG), co-poly (ethylene/propylene) glycol, polyoxyethylene (POE), polyurethane, polyphosphazene, polysaccharides, dextran, polyvinylpyrrolidones, polyvinyl ethyl ether, polyacryl amide, polyacrylate, or polycyanoacrylates.


SEQUENCE LISTING (48 sequences)

SEQ ID NO: 1 - protein, 7 aa, homo sapiens
TVRTSAD

SEQ ID NO: 2 - protein, 7 aa, Homo sapiens
NWGDRIL

SEQ ID NO: 3 - protein, 7 aa, Homo sapiens
NWGKPIK

SEQ ID NO: 4 - protein, 7 aa, Homo sapiens
SGVKSAF

SEQ ID NO: 5 - protein, 7 aa, Homo sapiens
GVKSYNE

SEQ ID NO: 6 - protein, 7 aa, Homo sapiens
IGKTNTL

SEQ ID NO: 7 - protein, 7 aa, Homo sapiens
IGNSNTL

SEQ ID NO: 8 - protein, 7 aa, Homo sapiens
IGNTIPV

SEQ ID NO: 9 - protein, 7 aa, Homo sapiens
LYANSPF

SEQ ID NO: 10 - protein, 9 aa, Homo sapiens
CTVRTSADC

SEQ ID NO: 11 - protein, 9 aa, Homo sapiens
CNWGDRILC

SEQ ID NO: 12 - protein, 9 aa, Homo sapiens
CNWGKPIKC

SEQ ID NO: 13 - protein, 9 aa, Homo sapiens
CSGVKSAFC

SEQ ID NO: 14 - protein, 9 aa, Homo sapiens
CGVKSYNEC

SEQ ID NO: 15 - protein, 9 aa, Homo sapiens
CIGKTNTLC

SEQ ID NO: 16 - protein, 9 aa, Homo sapiens
CIGNSNTLC

SEQ ID NO: 17 - protein, 9 aa, Homo sapiens
CIGNTIPVC

SEQ ID NO: 18 - protein, 9 aa, Homo sapiens
CLYANSPFC

SEQ ID NO: 19 - protein, 7 aa, Homo sapiens
WNYPFRL

SEQ ID NO: 20 - protein, 7 aa, Homo sapiens
SNTSYVN

SEQ ID NO: 21 - protein, 7 aa, Homo sapiens
SFSYTSG

SEQ ID NO: 22 - protein, 7 aa, Homo sapiens
WSPAPMS

SEQ ID NO: 23 - protein, 7 aa, Homo sapiens
TREHPAQ

SEQ ID NO: 24 - protein, 7 aa, Homo sapiens
ARIIDNA

SEQ ID NO: 25 - protein, 9 aa, Homo sapiens
CWNYPFRLC

SEQ ID NO: 26 - protein, 9 aa, Homo sapiens
CSNTSYVNC

SEQ ID NO: 27 - protein, 9 aa, Homo sapiens
CSFSYTSGC

SEQ ID NO: 28 - protein, 9 aa, Homo sapiens
CWSPAPMSC

SEQ ID NO: 29 - protein, 9 aa, Homo sapiens
CTREHPAQC

SEQ ID NO: 30 - protein, 9 aa, Homo sapiens
CARIIDNAC

SEQ ID NO: 31 - protein, 8 aa, Artificial Sequence
  Synthetic Construct
CTVRTSAD

SEQ ID NO: 32 - protein, 8 aa, Artificial Sequence
  Synthetic Construct
CNWGDRIL

SEQ ID NO: 33 - protein, 8 aa, Artificial Sequence
  Synthetic Construct
CNWGKPIK

SEQ ID NO: 34 - protein, 8 aa, Artificial Sequence
  Synthetic Construct
CSGVKSAF

SEQ ID NO: 35 - protein, 8 aa, Artificial Sequence
  Synthetic Construct
CGVKSYNE

SEQ ID NO: 36 - protein, 8 aa, Artificial Sequence
  Synthetic Construct
CIGKTNTL

SEQ ID NO: 37 - protein, 8 aa, Artificial Sequence
  Synthetic Construct
CIGNSNTL

SEQ ID NO: 38 - protein, 8 aa, Artificial Sequence
  Synthetic Construct
CIGNTIPV

SEQ ID NO: 39 - protein, 8 aa, Artificial Sequence
  Synthetic Construct
CLYANSPF

SEQ ID NO: 40 - protein, 8 aa, Artificial Sequence
  Synthetic Construct
CTVRTSAD

SEQ ID NO: 41 - protein, 8 aa, Artificial Sequence
  Synthetic Construct
CNWGDRIL

SEQ ID NO: 42 - protein, 8 aa, Artificial Sequence
  Synthetic Construct
CNWGKPIK

SEQ ID NO: 43 - protein, 8 aa, Artificial Sequence
  Synthetic Construcct
CSGVKSAF

SEQ ID NO: 44 - protein, 8 aa, Artificial Sequence
  Synthetic Construct
CGVKSYNE

SEQ ID NO: 45 - protein, 8 aa, Artificial Sequence
  Synthetic Construct
CIGKTNTL

SEQ ID NO: 46 - protein, 8 aa, Artificial Sequence
  Synthetic Construct
CIGNSNTL

SEQ ID NO: 47 - protein, 8 aa, Artificial Sequence
  Synthetic Construct
CIGNTIPV

SEQ ID NO: 48 - protein, 8 aa, Artificial Sequence
  Synthetic Construct
CLYANSPF